(19) United States
(12) Patent Application Publication
(10) Pub. No.: US 2003/0157525 A1
(43) Pub. Date: Aug. 21, 2003

(54) Novel human G-protein coupled receptor, HGPRBMY31, and variants and methods of use thereof

(76) Inventors: Gabriel A. Mintier, Hightstown, NJ (US); Chandra S. Ramanathan, Wallingford, CT (US); John N. Feder, Belle Mead, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/305,555

(22) Filed: Nov. 26, 2002

Related U.S. Application Data
(60) Provisional application No. 60/333,337, filed on Nov. 26, 2001.
(60) Provisional application No. 60/355,619, filed on Feb. 6, 2002.

(51) Int. Cl. C12Q 1/68; G01N 33/53; C12P 21/02; C12N 5/06; C07K 14/705

(52) U.S. Cl. 435/6; 435/69.1; 435/320.1; 435/325; 530/350; 536/23.5; 514/12; 435/7.1

(57) ABSTRACT

The present invention describes human G-protein coupled receptors (GPCRs) and their encoding polynucleotides. Also described are expression vectors, host cells, antisense molecules, and antibodies associated with the GPCR polynucleotides and/or polypeptides of this invention. In addition, methods for treating, diagnosing, preventing, and screening for disorders or diseases associated with abnormal biological activity of GPCR are described, as are methods for screening for modulators, for example, agonists or antagonists, of GPCR activity and/or function.

This application claims benefit to provisional application U.S. Serial No. 60/333,337 filed Nov. 26, 2001; and to provisional application U.S. Serial No. 60/355,619, filed Feb. 6, 2002. The entire teachings of the referenced applications are incorporated herein by reference.

### FIELD OF THE INVENTION

The present invention relates to novel G-protein coupled receptor (GPCR) nucleic acid or polynucleotide sequences which encode GPCR proteins. This invention further relates to fragments of novel GPCR nucleic acid sequences and their encoded amino acid sequences. Additionally, the invention relates to methods of using the GPCR polynucleotide sequences and encoded GPCR proteins for genetic screening and for the treatment of diseases, disorders, conditions, or syndromes associated with GPCRs.

### BACKGROUND OF THE INVENTION

Many medically significant biological processes that are mediated by proteins participating in signal transduction pathways involving G-proteins and/or second messengers, e.g., cAMP, have been established (Lefkowitz, Nature, 351:353-354 (1991)). These proteins are referred to herein as proteins participating in pathways with G-proteins or PPG proteins. Some examples of these proteins include the G protein-coupled receptors (GPCR), such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenylate cyclase, and phosphodiesterase, and actuator proteins, e.g., protein kinase A and protein kinase C (Simon, M. I., et al., Science, 252:802-8 (1991)).

For example, in one form of signal transduction, the effect of hormone binding results in activation of the enzyme adenylate cyclase inside the cell. Enzyme activation by hormones is dependent on the presence of the nucleotide GTP, where GTP also influences hormone binding. A G-protein binds the hormone receptors to adenylate cyclase. The G-protein has further been shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form. Thus, the G-protein serves a dual role—as an intermediate that relays the signal from receptor to effector, and as a “clock” that controls the duration of the signal.

The membrane protein gene superfamily of G-protein coupled receptors (GPCRs) has been characterized as having seven putative transmembrane domains. The domains are believed to represent transmembrane α-helices connected by extracellular or cytoplasmic loops. GPCRs include a wide range of biologically active receptors, such as hormone, viral, growth factor, and neuronal receptors.

GPCRs are further characterized as having seven conserved hydrophobic stretches of about 20 to 30 amino acids, connecting at least eight divergent hydrophilic loops. The G-protein family of coupled receptors includes dopamine receptors, which bind to neuroleptic drugs, used for treating psychotic and neurological disorders. Other examples of members of this family of receptors include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant and cytomegalovirus receptors, etc.

Most GPCRs have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some GPCRs. Most GPCRs contain potential phosphorylation sites within the third cytoplasmic loop and/or the carboxyl terminus. For several GPCRs, such as the β-adrenoreceptor, phosphorylation by protein kinase A and/or specific receptor kinases mediates receptor desensitization.

For some receptors, the ligand binding sites of GPCRs are believed to comprise a hydrophilic socket formed by the transmembrane domains of several GPCRs. This socket is surrounded by hydrophobic residues of the GPCRs. The hydrophilic side of each GPCR transmembrane helix is postulated to face inward and form the polar ligand-binding site. TM3 has been implicated in several GPCRs as having a ligand-binding site, which includes the TM3 aspartate residue. Additionally, TM5 serines, a TM6 asparagine and TM6 or TM7 phenylalanines or tyrosines are also implicated in ligand binding.

Recently, the function of many GPCRs has been shown to be enhanced upon dimerization and/or oligomerization of the activated receptor. In addition, sequestration of the activated GPCR appears to be altered upon the formation of multimeric complexes (AbdAlla, S., et al., Nature, 407:94-98 (2000)).

Structural biology has provided significant insight into the function of the various conserved residues found amongst numerous GPCRs. For example, the tripeptide Asp(Glu)-Arg-Tyr motif is important in maintaining the inactive confirmation of G-protein coupled receptors. The residues within this motif participate in the formation of several hydrogen bonds with surrounding amino acid residues that are important for maintaining the inactive state (Kim, J. M., et al., Proc. Natl. Acad. Sci. U.S.A., 94:14273-14278 (1997)). Another example relates to the conservation of two Leu (Leu76 and Leu79) residues found within helix II and two Leu residues (Leu 128 and Leu131) found within helix III of GPCRs. Mutation of the Leu128 results in a constitutively active receptor—emphasizing the importance of this residue in maintaining the ground state (Tao, Y. X., et al., Mol. Endocrinol., 14:1272-1282 (2000); and Lu. Z. L., and Hulme, E. C., J. Biol. Chem., 274:7309-7315 (1999). Additional information relative to the functional relevance of several conserved residues within GPCRs may be found by reference to Okada et al in Trends Biochem. Sci., 25:318-324 (2001).

GPCRs can be intracellularly coupled by heterotrimeric G-proteins to various intracellular enzymes, ion channels and transporters (see, Johnson et al., Endoc., Rev., 10:317-331(1989)). Different G-protein β-subunits preferentially stimulate particular effectors to modulate various biological functions in a cell. Phosphorylation of cytoplasmic residues of GPCRs have been identified as an important mechanism for the regulation of G-protein coupling of some GPCRs. GPCRs are found in numerous sites within a mammalian host.

GPCRs are one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). They have also been shown to play a role in HIV infection (Y. Feng et al., Science, 272: 872-877 (1996)). The structure of GPCRs consists of seven transmembrane helices that are connected by loops. The N-terminus is always extracellular and C-terminus is intracellular. GPCRs are involved in signal transduction. The signal is received at the extracellular N-terminus side. The signal can be an endogenous ligand, a chemical moiety or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists, for these GPCRs are used for therapeutic purposes.

### SUMMARY OF THE INVENTION

The present invention provides GPCR polynucleotides, preferably full-length, and their encoded polypeptides. The GPCR polynucleotides and polypeptides, may be involved in a variety of diseases, disorders and conditions associated with GPCR activity. More specifically, the present invention is concerned with the modulation of these GPCR polynucleotides and encoded products, particularly in providing treatments and therapies for relevant diseases. Antagonizing or inhibiting the action of the GPCR polynucleotides and polypeptides is especially encompassed by the present invention.

It is an object of this invention to provide isolated GPCR polynucleotides as depicted in SEQ ID NO:1. Another object of this invention is to provide GPCR polypeptides, encoded by the polynucleotide of SEQ ID NO:1 and having the encoded amino acid sequences of SEQ ID NO:2, respectively, or a functional or biologically active portion of these sequences.

It is yet another object of this invention to provide an isolated GPCR polynucleotide variant as depicted in SEQ ID NO:3. A further object of this invention is to provide a GPCR polypeptide, encoded by the polynucleotide of SEQ ID NO:3 and having the encoded amino acid sequences of SEQ ID NO:4, respectively, or a functional or biologically active portion of these sequences.

It is yet another object of the invention to provide compositions comprising the GPCR polynucleotide sequences, or fragments thereof, or the encoded GPCR polypeptides, or fragments or portions thereof. In addition, this invention provides pharmaceutical compositions comprising at least one GPCR polypeptide, or functional portion thereof, wherein the compositions further comprise a pharmaceutically and physiologically acceptable carrier, excipient, or diluent.

A further embodiment of this invention presents polynucleotide sequences comprising the complement of SEQ ID NO:1 and 3, or variants thereof. In addition, an object of the invention encompasses variations or modifications of the GPCR sequences which are a result of degeneracy of the genetic code, where the polynucleotide sequences can hybridize under moderate or high stringency conditions to the polynucleotide sequences of SEQ ID NO:1 and 3.

It is another object of the invention to provide nucleic acid sequences encoding the novel GPCR polypeptides and antisense of the nucleic acid sequences, as well as oligonucleotides, fragments, or portions of the nucleic acid molecules or antisense molecules. Also provided are expression vectors and host cells comprising polynucleotides that encode the GPCR polypeptides.

A further object of the present invention encompasses amino acid sequences encoded by the novel GPCR nucleic acid sequences. The amino acid sequences of SEQ ID NO:2 and 4 are encoded by the nucleic acid sequences SEQ ID NO:1 and 3, respectively. More specifically, these GPCR polypeptides are of several types, namely, sensory GPCRs, orphan GPCRs, chemokine GPCRs, or very large GPCRs. GPCRs have been described in relation to dopamine receptors, rhodopsin receptors, kinin receptors, N-formyl peptide receptors, opioid receptors, calcitonin receptors, adrenergic receptors, endothelin receptors, cAMP receptors, adenosine receptors, muscarinic receptors, acetylcholine receptors, serotonin receptors, histamine receptors, thrombin receptors, follicle stimulating hormone receptors, opsin receptors, endothelial differentiation gene-1 receptors, odorant receptors, and cytomegalovirus receptors.

In yet another object, the present invention provides pharmaceutical compositions comprising the GPCR polynucleotide sequences, or fragments thereof, or the encoded GPCR polypeptide sequences, or fragments or portions thereof. Also provided are pharmaceutical compositions comprising GPCR polypeptide sequences, homologues, or one or more functional portions thereof, wherein the compositions further comprise a pharmaceutically- and/or physiologically-acceptable carrier, excipient, or diluent. All fragments or portions of the GPCR polynucleotides and polypeptides are preferably functional or active.

Another object of the invention is to provide methods for producing a polypeptide comprising the amino acid sequences of SEQ ID NO:2 and 4, or a fragment thereof, preferably, a functional fragment or portion thereof, comprising the steps of a) cultivating a host cell containing an expression vector containing at least a functional fragment of the polynucleotide sequence encoding the GPCR proteins according to this invention under conditions suitable for the expression of the polypeptide; and b) recovering the polypeptide from the host cell.

Another object of this invention is to provide a substantially purified modulator, preferably an antagonist or inhibitor, of one or more of the GPCR polypeptides having SEQ ID NO:2 and 4. In this regard, and by way of example, a purified antibody, or antigenic epitope thereof that binds to a polypeptide comprising the amino acid sequence of SEQ ID NO:2 and 4, or homologue encoded by a polynucleotide having a nucleic acid sequence, or degenerate thereof, as set forth in any one of SEQ ID NO:1 and 3, is provided.

It is yet another object of the present invention to provide GPCR nucleic acid sequences, polypeptides, peptides and antibodies for use in the diagnosis and/or screening of disorders or diseases associated with expression of one or more of the GPCR polynucleotides and their encoded polypeptide products as described herein.

Another object of this invention is to provide diagnostic probes or primers for detecting GPCR-related diseases and/or for monitoring a patient's response to therapy. The probe or primer sequences comprise nucleic acid or amino acid sequences of the GPCRs described herein.

It is another object of the present invention to provide a method for detecting a polynucleotide that encodes a described GPCR polypeptide in a biological sample comprising the steps of: a) hybridizing the complement of the polynucleotide sequence encoding SEQ ID NO:1 and 3 to the nucleic acid material of a biological sample, thereby forming a hybridization complex; and b) detecting the hybridization complex, wherein the presence of the complex correlates with the presence of a polynucleotide encoding a GPCR polypeptide in the biological sample. The nucleic acid material may be further amplified by the polymerase chain reaction prior to hybridization.

Another object of this invention is to provide methods for screening for agents which modulate GPCR polypeptides, e.g., agonists and antagonists, particularly those that are obtained from the screening methods as described.

As yet a further object, the invention provides methods for detecting genetic predisposition, susceptibility and response to therapy of various GPCR-related diseases, disorders, or conditions.

It is another object of the present invention to provide methods for the treatment or prevention of several GPCR-associated diseases or disorders including, but not limited to, cancers, and/or cardiovascular, immune, or neurological diseases or disorders. The methods involve administering to an individual in need of such treatment or prevention an effective amount of a purified antagonist of one or more of a GPCR polypeptide.

The invention further relates to a method for preventing, treating, or ameliorating a medical condition with the polypeptide provided as SEQ ID NO:2 or SEQ ID NO:4, in addition to, its encoding nucleic acid, or a modulator thereof, wherein the medical condition is an immune disorder, hematopoietic disorder, reproductive disorder, a disorder related to aberrant T-cell maturation, leukemia, multiple myeloma, related proliferative condition of the immune system, neural disorder, brain cancer, related proliferative condition of the central nervous system, renal disorder, bladder disorder, and urinary incontinence.

The invention further relates to a method of diagnosing a pathological condition or a susceptibility to a pathological condition in a subject comprising the steps of (a) determining the presence or amount of expression of the polypeptide of SEQ ID NO:2 or SEQ ID NO:4 in a biological sample; (b) and diagnosing a pathological condition or a susceptibility to a pathological condition based on the presence or amount of expression of the polypeptide relative to a control, wherein said condition is a member of the group consisting of an immune disorder, hematopoietic disorder, reproductive disorder, a disorder related to aberrant T-cell maturation, leukemia, multiple myeloma, related proliferative condition of the immune system, neural disorder, brain cancer, related proliferative condition of the central nervous system, renal disorder, bladder disorder, and urinary incontinence.

In preferred embodiments, HGPRBMY31 polynucleotides and polypeptides including agonists and fragments thereof, have uses which include treating, diagnosing, prognosing, and/or preventing neural disorders, hypersensitivity disorders, particularly pain disorders, or any neural disorder related to either a direct or indirect interaction with the various voltage-gated sodium channel and their beta subunits as these channels function to the make the DGR neuron hyper-excitable following injury to the nervous system.

In preferred embodiments, HGPRBMY31 polynucleotides and polypeptides including agonists and fragments thereof, have uses which include treating, diagnosing, prognosing, and/or preventing neural disorders, particularly neural disorders related to aberrations or injuries in the cerebellum, including, but not limited to, cerebellar ataxias of known and unknown origin such as Coeliac disease, and other diseases associated with this region of the brain such as, Rett syndrome, Parkinson disease, von Hippel-Lindau syndrome, familial congenital cerebellar hypoplasia, and dysplastic gangliocytoma of cerebellum.

In preferred embodiments, HGPRBMY31 polynucleotides and polypeptides including agonists and fragments thereof, have uses which include treating, diagnosing, prognosing, and/or preventing urinary or renal diseases or disorders, such as, for example, incontinence, including urinary incontinence caused by prostatectomy and over-active bladder.

In preferred embodiments, the HGPRBMY31 polynucleotides and polypeptides, including agonists, antagonists, and fragments thereof, are useful for modulating intracellular cAMP levels, modulating cAMP sensitive signaling pathways, and modulating CRE element associated signaling pathways.

It is yet another object of this invention to provide diagnostic kits for the determination of the nucleotide sequences of human GPCR alleles. The kits can comprise reagents and instructions for amplification-based assays, nucleic acid probe assays, protein nucleic acid probe assays, antibody assays or any combination thereof. Such kits are suitable for screening and the diagnosis of disorders associated with aberrant or uncontrolled cellular development and with the expression of one or more GPCR polynucleotide and encoded GPCR polypeptide as described herein.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO-K1 or HEK 293 cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO-K1 or HEK 293 cells that comprise a vector comprising the coding sequence of the luciferase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO-K1 or HEK 293 cells that comprise a vector comprising the coding sequence of the luciferase gene under the control of CRE response elements, wherein said cells express luciferase at high, moderate, or low levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO-K1 or HEK 293 cells that comprise a vector comprising the coding sequence of the luciferase gene under the control of CRE response elements, and wherein said method optionally comprises the addition of Forskolin.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO-K1 or HEK 293 cells that comprise a vector comprising the coding sequence of the luciferase gene under the control of CRE response elements, wherein said method optionally comprises the addition of Forskolin, and wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are HEK cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, and futher wherein said cells express the polypeptide at either low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said cells express beta lactamase at low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-3949, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said cells express beta lactamase at low, moderate, or high levels.

Further objects, features, and advantages of the present invention will be better understood upon a reading of the detailed description of the invention when considered in connection with the accompanying figures or drawings.

### BRIEF DESCRIPTION OF THE FIGURES

FIGS. 1A-D show the polynucleotide sequence (SEQ ID NO:1) and deduced amino acid sequence (SEQ ID NO:2) of the novel human G-protein coupled receptor, HGPRBMY31, of the present invention. The standard one-letter abbreviation for amino acids is used to illustrate the deduced amino acid sequence. The polynucleotide sequence contains a sequence of 3791 nucleotides (SEQ ID NO:1), encoding a polypeptide of 307 amino acids (SEQ ID NO:2). An analysis of the HGPRBMY31 polypeptide determined that it comprised the following features—six transmembrane domains (TM1 to TM6) located from about amino acid 28 to about amino acid 49 (TM1); from about amino acid 61 to about amino acid 79 (TM2); from about amino acid 105 to about amino acid 127 (TM3); from about amino acid 141 to about amino acid 164 (TM4); from about amino acid 186 to about amino acid 205 (TM5); from about amino acid 219 to about amino acid 242 (TM6); and/or from about amino acid 255 to about amino acid 278 (TM7) of SEQ ID NO:2 (FIGS. 1A-D) represented by double underlining. It is anticipated that the HGPRBMY31 polypeptide may function as a G-protein coupled receptor as described more particularly elsewhere herein.

FIG. 2 presents the nucleic acid sequence (SEQ ID NO:3) of a novel human GPCR, HGPRBMY31 variant.

FIG. 3 illustrates an alignment of the novel human class A HGPRBMY31 (Q; Query) with the target protein Pfam model (a transmembrane receptor of the rhodopsin family; T; Target) using the protein sequence database and BLAST analysis as known and as described herein. FIG. 3 illustrates the domain prediction for the GPCR encoded by HGPRBMY31, where amino acids 44-80 of the Q sequence of domain 1 (SEQ ID NO:24) is aligned with amino acids 1-37 of the T sequence of domain 1 (SEQ ID NO:25). Domain 2 of the Q sequence ranges from amino acids 104-275 (SEQ ID NO:26), and is aligned with that of the T sequence from amino acids 65-256 (SEQ ID NO:27).

FIG. 4 illustrates an alignment of the novel human class A HGPRBMY31 variant (Q; Query) with the target protein Pfam model (T; Target) using the protein sequence database and BLAST analysis as known and as described herein. FIG. 4 illustrates the domain prediction for the GPCR encoded by HGPRBMY31 variant, where amino acids 44-80 of the Q sequence of domain 1 (SEQ ID NO:28) is aligned with amino acids 1-37 of the T sequence of domain I (SEQ ID NO:29). Domain 2 of the Q sequence ranges from amino acids 104-276 (SEQ ID NO:30), and is aligned with that of the T sequence from amino acids 65-259 (SEQ ID NO:31).

FIGS. 5A-B presents the multiple sequence alignment of the translated sequence of the G-protein coupled receptor, HGPRBMY31, where the GCG pileup program was used to generate the alignment. The blackened areas represent identical amino acids in more than half of the listed sequences and the grey highlighted areas represent similar amino acids. As shown in FIGS. 10A-10B, the sequences are aligned according to their amino acids, where: HGPRBMY31 (SEQ ID NO:2) is the translated full length HGPRBMY31 cDNA; HGPRBMY31 variant (SEQ ID NO:4) is the translated full length HGPRBMY31_variant; C5AR_CAVPO (SEQ ID NO:7; SWISS-PROT Accession No. 070129) is the Cavia porcellus C5A anaphylatoxin chemotactic receptor; FML2_PONPY (SEQ ID NO:8; SWISS-PROT Accession No. P79237) is the Pongo pygmaeus orangutan N-formyl peptide receptor-like 2 receptor fragment; GPRO90_MOUSE (SEQ ID NO:9; Genbank Accession No. gi|13507682) is the Mus musculus G-protein coupled receptor GPR90; MAS_HUMAN (SEQ ID NO:10; SWISS-PROT Accession No. P04201) is the human Mas proto-oncogene; MAS_MOUSE (SEQ ID NO:11; SWISS-PROT Accession No. P30554) is the Mus musculus Mas proto-oncogene; MAS_RAT (SEQ ID NO:12; SWISS-PROT Accession No. P12526) is the Rattus norvegicus Mas proto-oncogene; MRG_HUMAN (SEQ ID NO:13; SWISS-PROT Accession No. P35410) is the human Mas-related G protein-coupled receptor, MRG; RTA_RAT (SEQ ID NO:14; SWISS-PROT Accession No. P23749) is the probable Rattus norvegicus G protein-coupled receptor, RTA; and ORPHAN_MOUSE (SEQ ID NO:15; Genbank Accession No. gi|12853220) is the Mus musculus putative G protein-coupled receptor.

FIG. 6 shows the expression profiling of the novel human class A GPCR, HGPRBMY31, as described in Example 4.

FIG. 7 shows an expanded expression profile of the novel human class A GPCR, HGPRBMY31, as described in Example 5.

FIG. 8 demonstrates that HGPRBMY31 couples to the cAMP second messenger pathway in CHO-K1 cells as measured by a CRE-luciferase reporter. CHO-K1 cells were transiently co-transfected with either the HGPRBMY31/pEF-DEST51™ mammalian expression construct (“CRE-Luc/BMY31”) or pEF-DEST51™ vector alone (“CRE-Luc/Vector”), in addition to the pCRE-Luciferase reporter construct (Stratagene) as described in Example 6. Constitutive trans-gene expression of HGPRBMY31 results in a marked decrease in cAMP relative to the control, as measured by the CRE-Luc reporter. Stimulation HGPRBMY31 co-transfected cells by the adenylate cyclase activator forskolin results in a significant reduction in cAMP accumulation when compared to cells transfected with vector alone. Both results are consistent with HGRBMY31 representing a functional G-protein coupled receptor that couples via the cAMP second messenger pathway through the G alpha i/o family of G-proteins.

FIG. 9 demonstrates that HGPRBMY31 couples to the cAMP second messenger in HEK 293 cells as measured by a CRE-luciferase reporter. HEK 293 cells were transiently co-transfected with either the HGPRBMY31/pEF-DEST51™ mammalian expression construct (“CRE-Luc/BMY31”) or pEF-DEST51™ vector alone (“CRE-Luc/Vector”), in addition to the pCRE-Luciferase reporter construct (Stratagene) as described in Example 6. In the absence, as well as in the presence of forskolin, there is a definitive decrease in cAMP levels in cells expressing HGPRBMY31 polypeptide relative to the control as measured by CRE-Luciferase. The elucidation of HGPRBMY31 effecting the cAMP pathway in both CHO-K1 and HEK 293 cells further demonstrates that HGPRBMY31 functionally couples via the cAMP second messenger pathway through the G alpha i/o family of G-proteins.

Table I provides a summary of various conservative substitutions encompassed by the present invention.

Table II provides a summary of the novel polypeptides and their encoding polynucleotides of the present invention.

### DETAILED DESCRIPTION OF THE INVENTION

The present invention provides novel human GPCR (GPCR) genes (i.e., polynucleotide or nucleic acid sequences) which encode GPCR proteins (polypeptides), preferably full-length GPCR polypeptides. Specifically, the present invention relates to novel HGPRBMY31 polynucleotides and polypeptides. The invention also relates to the polynucleotides and polypeptides of a novel HGPRBMY31 splice variant. The invention further relates to fragments and portions of novel GPCR nucleic acid sequences and their encoded amino acid sequences (peptides). Preferably, the fragments and portions of the GPCR polypeptides are functional or active. The invention also provides methods of using the novel GPCR polynucleotide sequences and the encoded GPCR polypeptides for genetic screening and for the treatment of diseases, disorders, conditions, or syndromes associated with GPCRs and GPCR activity and function. All references to “HGPRBMY31” shall be construed to apply to “HGPRBMY31” and/or the “HGPRBMY31 splice variant”, unless otherwise specified herein.

### DEFINITIONS

The following definitions are provided to more fully describe the present invention in its various aspects. The definitions are intended to be useful for guidance and elucidation, and are not intended to limit the disclosed invention or its embodiments.

“Amino acid sequence” as used herein can refer to an oligopeptide, peptide, polypeptide, or protein sequence, and fragments or portions thereof, as well as to naturally occurring or synthetic molecules, preferably isolated polypeptides of the GPCR. Amino acid sequence fragments are typically from about 4 to about 30, preferably from about 5 to about 15, more preferably from about 5 to about 15 amino acids in length and preferably retain the biological activity or function of a GPCR polypeptide. GPCR amino acid sequences of this invention are set forth in SEQ ID NO:2 and 4 and in description of the Figures. The terms GPCR polypeptide and GPCR protein are used interchangeably herein to refer to the encoded products of the GPCR nucleic acid sequences according to the present invention.

Isolated GPCR polypeptide refers to the amino acid sequence of substantially purified GPCR, which may be obtained from any species, preferably mammalian, and more preferably, human, and from a variety of sources, including natural, synthetic, semi-synthetic, or recombinant. More particularly, the GPCR polypeptides of this invention are identified in SEQ ID NO:2 and 4. Functional fragments of the GPCR polypeptides are also embraced by the present invention.

As will be appreciated by the skilled practitioner, should the amino acid fragment comprise an antigenic epitope, for example, biological function per se need not be maintained. The terms HGPRBMY31 polypeptide and HGPRBMY31 protein are used interchangeably herein to refer to the encoded product of the HGPRBMY31 nucleic acid sequence according to the present invention.

“Similar” amino acids are those which have the same or similar physical properties and in many cases, the function is conserved with similar residues. For example, amino acids lysine and arginine are similar; while residues such as proline and cysteine do not share any physical property and are not considered to be similar.

The term “consensus” refers to a sequence that reflects the most common choice of base or amino acid at each position among a series of related DNA, RNA or protein sequences. Areas of particularly good agreement often represent conserved functional domains.

A “variant” of a GPCR polypeptide refers to an amino acid sequence that is altered by one or more amino acids. The variant may have “conservative” changes, in which a substituted amino acid has similar structural or chemical properties, for example, replacement of leucine with isoleucine. More rarely, a variant may have “non-conservative” changes, for example, replacement of a glycine with a tryptophan. The encoded protein may also contain deletions, insertions, or substitutions of amino acid residues, which produce a silent change and result in a functionally equivalent GPCR protein. Deliberate amino acid substitutions may be made on the basis of similarity in polarity, charge, solubility, hydrophobicity, hydrophilicity, and/or the amphipathic nature of the residues, as long as the biological activity of GPCR protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine. Guidance in determining which amino acid residues may be substituted, inserted, or deleted without abolishing functional biological or immunological activity may be found using computer programs well known in the art, for example, DNASTAR, Inc. software (Madison, Wis.).

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1990).

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in Table I below.

TABLE 1

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, |
| | | cis-3,4, or 5-phenylproline |
| Proline | P | D-Pro, L-1-thioazolidine-4-carboxylic acid, |
| | | D- or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., β or γ amino acids.

Both identity and similarity can be readily calculated by reference to the following publications: Computational Molecular Biology, Lesk, A.M., ed., Oxford University Press, New York, 1988; Biocomputing: Informatics and Genome Projects, Smith, D. W., ed., Academic Press, New York, 1993; Informatics Computer Analysis of Sequence Data, Part 1, Griffin, A.M., and Griffin, H. G., eds., Humana Press,New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991.

In addition, the present invention also encompasses substitution of amino acids based upon the probability of an amino acid substitution resulting in conservation of function. Such probabilities are determined by aligning multiple genes with related function and assessing the relative penalty of each substitution to proper gene function. Such probabilities are often described in a matrix and are used by some algorithms (e.g., BLAST, CLUSTALW, GAP, etc.) in calculating percent similarity wherein similarity refers to the degree by which one amino acid may substitute for another amino acid without lose of function. An example of such a matrix is the PAM250 or BLOSUM62 matrix.

Aside from the canonical chemically conservative substitutions referenced above, the invention also encompasses substitutions which are typically not classified as conservative, but that may be chemically conservative under certain circumstances. Analysis of enzymatic catalysis for proteases, for example, has shown that certain amino acids within the active site of some enzymes may have highly perturbed pKa's due to the unique microenvironment of the active site. Such perturbed pKa's could enable some amino acids to substitute for other amino acids while conserving enzymatic structure and function. Examples of amino acids that are known to have amino acids with perturbed pKa's are the Glu-35 residue of Lysozyme, the Ile-16 residue of Chymotrypsin, the His-159 residue of Papain, etc. The conservation of function relates to either anomalous protonation or anomalous deprotonation of such amino acids, relative to their canonical, non-perturbed pKa. The pKa perturbation may enable these amino acids to actively participate in general acid-base catalysis due to the unique ionization environment within the enzyme active site. Thus, substituting an amino acid capable of serving as either a general acid or general base within the microenvironment of an enzyme active site or cavity, as may be the case, in the same or similar capacity as the wild-type amino acid, would effectively serve as a conservative amino substitution.

The term “mimetic”, as used herein, refers to a molecule, having a structure which is developed from knowledge of the structure of a GPCR protein, or portions thereof, and as such, is able to affect some or all of the actions of the GPCR protein. A mimetic may comprise of a synthetic peptide or an organic molecule.

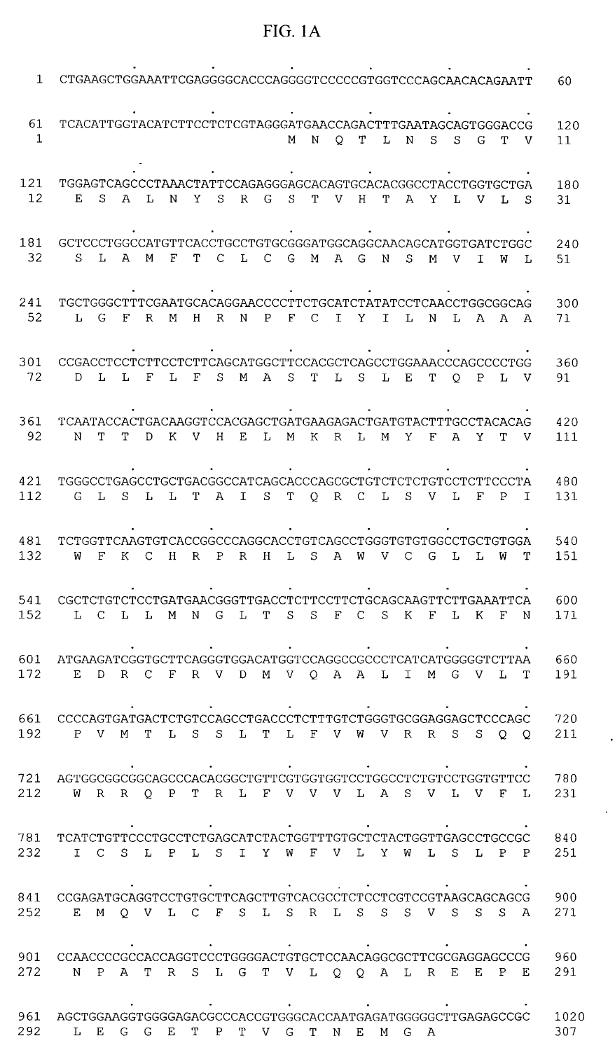

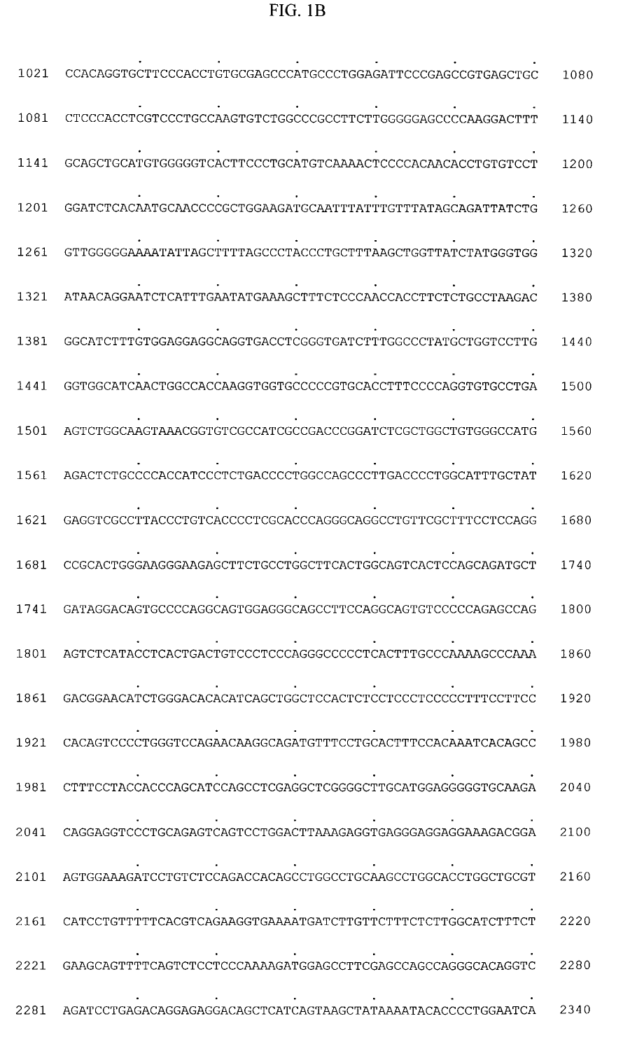

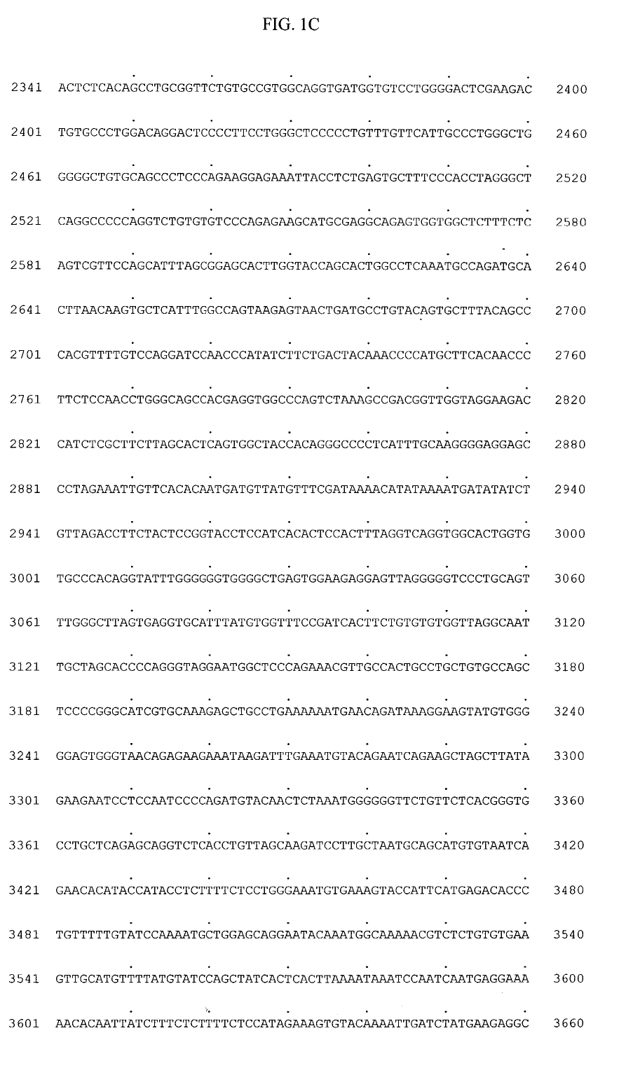

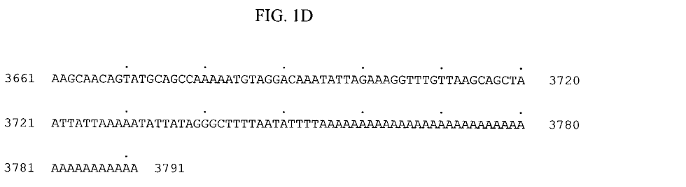

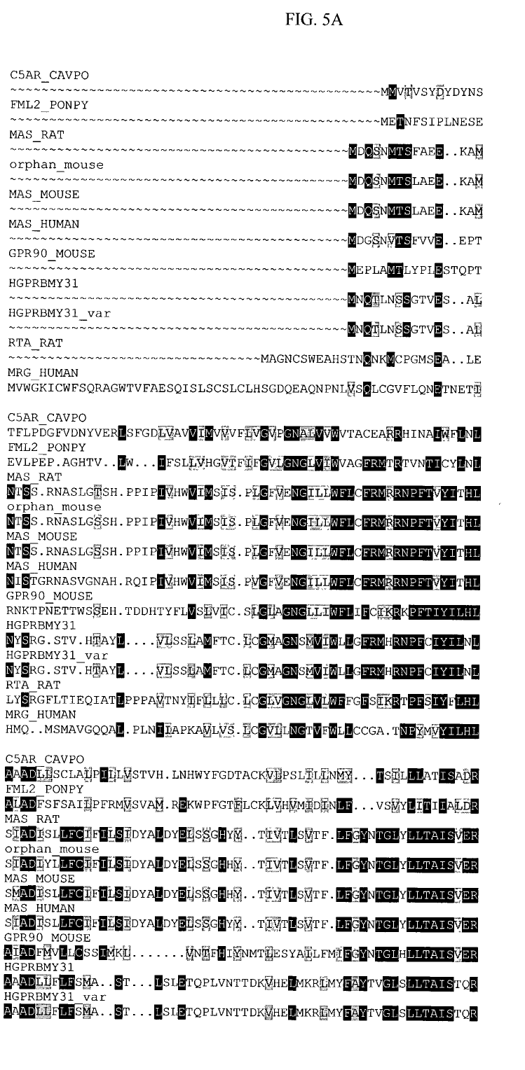

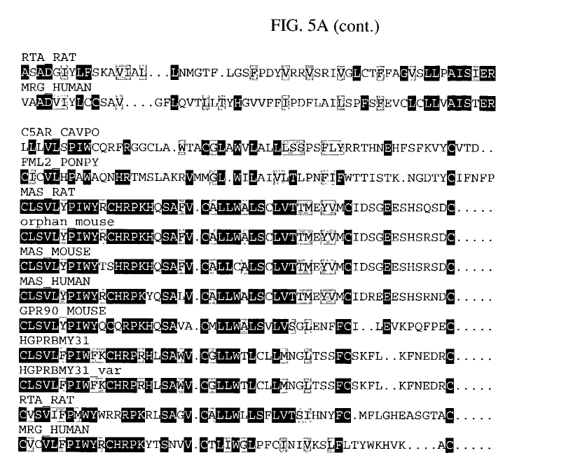

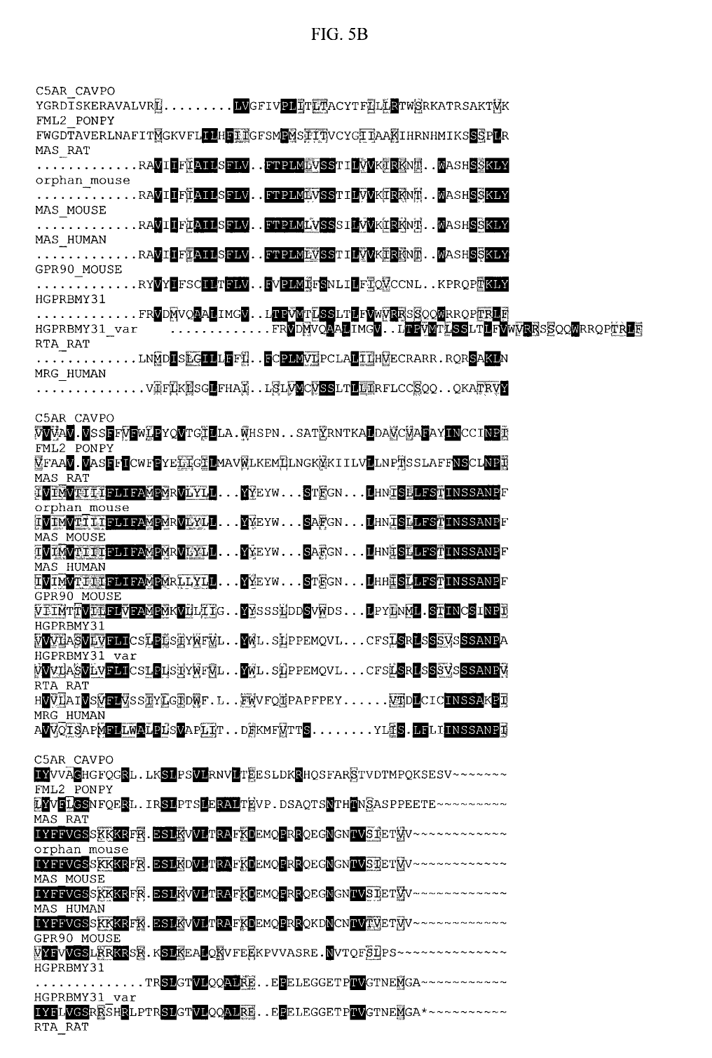

The phrases “nucleic acid” or “polynucleotide sequence”, as used herein, refer to an isolated oligonucleotide (“oligo”), nucleotide, or polynucleotide, and fragments thereof, and to DNA or RNA of genomic or synthetic origin which may be single- or double-stranded, and represent the sense or anti-sense strand, preferably of the GPCR. By way of non-limiting examples, fragments include nucleic acid sequences that are greater than 20-60 nucleotides in length, and preferably include fragments that are at least 70-100 nucleotides, or which are at least 1000 nucleotides or greater in length. GPCR nucleic acid sequences of this invention are specifically identified in SEQ ID NO:1 and 3 and as illustrated in FIGS. 1 and 5.

An “allele” or “allelic sequence” is an alternative form of a GPCR nucleic acid sequence. Alleles may result from at least one mutation in a GPCR nucleic acid sequence and may yield altered mRNAs or polypeptides whose structure or function may or may not be altered. Any given gene, whether natural or recombinant, may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

“Peptide nucleic acid” (PNA) refers to an antisense molecule or anti-gene agent which comprises an oligonucleotide linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. Such amino acids include, for example, lysine and arginine, among others. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers”, as defined herein, refer to a GPCR nucleic acid sequence comprising contiguous nucleotides, of at least about 5 nucleotides to about 60 nucleotides, preferably at least about 8 to 10 nucleotides in length, more preferably at least about 12 nucleotides in length, for example, about 15 to 35 nucleotides, or about 15 to 25 nucleotides, or about 20 to 35 nucleotides, which can be typically used in PCR amplification assays, hybridization assays, or in microarrays. It will be understood that the term oligonucleotide is substantially equivalent to the terms primer, probe, or amplimer, as commonly defined in the art.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNAs and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand. Antisense oligonucleotides may be single or double stranded. Double stranded RNA's may be designed based upon the teachings of Paddison et al., Proc. Nat. Acad. Sci., 99:1443-1448 (2002); and International Publication Nos. WO 01/29058, and WO 99/32619; which are hereby incorporated herein by reference.

“Altered” nucleic acid sequences encoding a GPCR polypeptide include nucleic acid sequences containing deletions, insertions and/or substitutions of different nucleotides resulting in a polynucleotide that encodes the same or a functionally equivalent GPCR polypeptide. Altered nucleic acid sequences may further include polymorphisms such as, single nucleotide polymorphism (SNPs), of the polynucleotide encoding a GPCR polypeptide. Such polymorphisms may or may not be readily detectable using a particular oligonucleotide probe.

The terms “Expressed Sequence Tag” or “EST” refers to the partial sequence of a cDNA insert which has been made by reverse transcription of mRNA extracted from a tissue, followed by insertion into a vector as known in the art (Adams, M. D., et al. Science (1991) 252:1651-1656; Adams, M. D. et al., Nature, (1992) 355:632-634; Adams, M. D., et al., Nature (1995) 377 Supp:3-174).

The term “biologically active”, i.e., functional, refers to a protein or polypeptide or fragment thereof, having structural, regulatory, or biochemical functions of a naturally occurring molecule. Likewise, “immunologically active” refers to the capability of a natural, recombinant, or synthetic GPCR, or an oligopeptide thereof, to induce a specific immune response in appropriate animals or cells, for example, to generate antibodies, to bind with specific antibodies, and/or to elicit a cellular immune response.

An “agonist” refers to a molecule which, when bound to, or associated with, a GPCR polypeptide, or a functional fragment thereof, increases or prolongs the duration of the effect of the GPCR polypeptide. Agonists may include proteins, nucleic acids, carbohydrates, or any other molecules that bind to and modulate the effect of GPCR polypeptide. Agonists typically enhance, increase, or augment the function or activity of a GPCR molecule.

An “antagonist” refers to a molecule which, when bound to, or associated with, a GPCR polypeptide, or a functional fragment thereof, decreases the amount or duration of the biological or immunological activity of GPCR polypeptide. Antagonists may include proteins, nucleic acids, carbohydrates, antibodies, or any other molecules that decrease or reduce the effect of a GPCR polypeptide. Antagonists typically, diminish, inhibit, or reduce the function or activity of a GPCR molecule.

It is another aspect of the present invention to provide modulators of the HGPRBMY31 protein and HGPRBMY31 peptide targets which can affect the function or activity of HGPRBMY31 in a cell in which HGPRBMY31 function or activity is to be modulated or affected. In addition, modulators of HGPRBMY31 can affect downstream systems and molecules that are regulated by, or which interact with, HGPRBMY31 in the cell. Modulators of HGPRBMY31 include compounds, materials, agents, drugs, and the like, that antagonize, inhibit, reduce, block, suppress, diminish, decrease, or eliminate HGPRBMY31 function and/or activity. Such compounds, materials, agents, drugs and the like can be collectively termed “antagonists”. Alternatively, modulators of HGPRBMY31 include compounds, materials, agents, drugs, and the like, that agonize, enhance, increase, augment, or amplify HGPRBMY31 function in a cell. Such compounds, materials, agents, drugs and the like can be collectively termed “agonists”.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The terms “complementary” or “complementarity” refer to the natural binding of polynucleotides under permissive salt and temperature conditions by base pairing. For example, the sequence “A-G-T” binds to the complementary sequence “T-C-A”. Complementarity between two single-stranded molecules may be “partial”, in which only some of the nucleic acids bind, or it may be “complete” when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to as the functional term “substantially homologous”. The inhibition of hybridization of the completely complementary sequence to the target sequence may be examined using a hybridization assay (for example, Southern or Northern blot, solution hybridization, and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (for example, less than about 30% identity). In the absence of non-specific binding, the probe will not hybridize to the second non-complementary target sequence.

Those having skill in the art will know how to determine percent identity between/among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the percent identity. CLUSTALW, however, does take sequence gaps into account in its identity calculations.

As a practical matter, whether any particular nucleic acid molecule or polypeptide is at least about 80%, 85%, 90%, 91%, 92%, 93%, 94%, 95%, 95.4%, 95.6%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 90%, 91%, 92%, 93%, 94%, 95%, 95.4%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced in Table 1 (SEQ ID NO:2) or to the amino acid sequence encoded by cDNA contained in a deposited clone, can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both amino acid sequences. The result of said global sequence alignment is in percent identity. Preferred parameters used in a CLUSTALW alignment of polypeptide sequences to calculate percent identity via pairwise alignments are: Matrix=BLOSUM, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global polypeptide alignments are often preferred since they reflect the percent identity between the polypeptide molecules as a whole (i.e., including any polypeptide overhangs, not just overlapping regions), as opposed to, only local matching polypeptides. Manual corrections for global percent identity determinations are required since the CLUSTALW program does not account for N- and C-terminal truncations of the subject sequence when calculating percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more polynucleotide or polypeptide sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modifed CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

Also available to those having skill in this art are the BLAST and BLAST 2.0 algorithms (Altschul et al., 1977, Nuc. Acids Res., 25:3389-3402 and Altschul et al., 1990, J. Mol. Biol., 215:403-410). The BLASTN program for nucleic acid sequences uses as defaults a wordlength (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff & Henikoff, 1989, Proc. Natl. Acad. Sci., USA, 89:10915) uses alignments (B) of 50, expectation (E) of 10, M=5, N=4, and a comparison of both strands.

The term “hybridization” refers to any process by which a strand of nucleic acids binds with a complementary strand through base pairing. The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (for example, Cₒt or Rₒt analysis), or between one nucleic acid sequence present in solution and another nucleic acid sequence immobilized on a solid phase or support (for example, membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt, and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (for example, formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency that is different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tₘ, can be approximated by the formulas as well known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G.M. Wahl and S. L. Berger (1987; Methods Enzymol. 152:399-407); and A. R. Kimmel, 1987; Methods of Enzymol. 152:507-511).

As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, for example, high, moderate, or low stringency, typically relates to such washing conditions. It is to be understood that the low, moderate and high stringency hybridization or washing conditions can be varied using a variety of ingredients, buffers and temperatures well known to and practiced by the skilled artisan.

A “composition”, as defined herein, refers broadly to any composition containing a GPCR polynucleotide, polypeptide, derivative, or mimetic thereof, or antibodies thereto. The composition may comprise a dry formulation or an aqueous solution. Compositions comprising GPCR polynucleotide sequences (SEQ ID NO:1 and 3) encoding GPCR polypeptides (SEQ ID NO:2 and 4), or fragments thereof, may be employed as hybridization probes. The probes may be stored in a freeze-dried form and may be in association with a stabilizing agent such as a carbohydrate. In hybridizations, the probe may be employed in an aqueous solution containing salts (for example, NaCl), detergents or surfactants (for example, SDS) and other components (for example, Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% to 95%, or greater, free from other components with which they are naturally associated.

The term “sample”, or “biological sample”, is meant to be interpreted in its broadest sense. A non-limiting example of a biological sample suspected of containing a GPCR nucleic acid encoding GPCR protein, or fragments thereof, or a GPCR protein itself, may comprise, but is not limited to, a body fluid, an extract from cells or tissue, chromosomes isolated from a cell (for example, a spread of metaphase chromosomes), organelle, or membrane isolated from a cell, a cell, nucleic acid such as genomic GPCR DNA (in solution or bound to a solid support such as, for example, for Southern analysis), GPCR RNA (in solution or bound to a solid support such as for Northern analysis), GPCR cDNA (in solution or bound to a solid support), a tissue, a tissue print, and the like.

“Transformation” or transfection refers to a process by which exogenous DNA, preferably GPCR, enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “correlates with expression of a polynucleotide” indicates that the detection of the presence of ribonucleic acid that is similar to the nucleic acid sequence of GPCRs by Northern analysis is indicative of the presence of mRNA encoding GPCR polypeptides (SEQ ID NO:2 and 4) in a sample and thereby correlates with expression of the transcript from the polynucleotide encoding the protein.

An alteration in the polynucleotide of SEQ ID NO:1 and 3 comprises any alteration in the sequence of the polynucleotides encoding GPCR polypeptides, including deletions, insertions, and point mutations that may be detected using hybridization assays. Included within this definition is the detection of alterations to the genomic DNA sequence which encodes GPCR polypeptides (e.g., by alterations in the pattern of restriction fragment length polymorphisms capable of hybridizing to nucleic acid sequences SEQ ID NO:1 and 3), the inability of a selected fragment of SEQ ID NO:1 and 3 to hybridize to a sample of genomic DNA (e.g., using allele-specific oligonucleotide probes), and improper or unexpected hybridization, such as hybridization to a locus other than the normal chromosomal locus for the polynucleotide sequence encoding GPCR polypeptide (e.g., using fluorescent in situ hybridization (FISH) to metaphase chromosome spreads).

The term “antibody” refers to intact molecules as well as fragments thereof, such as Fab, F(ab′)₂, Fv, or Fc which are capable of binding an epitopic or antigenic determinant. Antibodies that bind to GPCR polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (for example, a mouse, a rat, or a rabbit).

The term “humanized” antibody refers to antibody molecules in which amino acids have been replaced in the non-antigen binding regions (i.e., framework regions) of the immunoglobulin in order to more closely resemble a human antibody, while still retaining the original binding capability, for example, as described in U.S. Pat. No. 5,585,089 to C. L. Queen et al. In the present instance, humanized antibodies are preferably anti-GPCR specific antibodies.

The term “antigenic determinant” refers to that portion of a molecule that makes contact with a particular antibody (i.e., an epitope). When a protein or fragment of a protein, preferably a GPCR protein, is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide, preferably a GPCR protein, and a binding molecule, such as an agonist, an antagonist, or an antibody. The interaction is dependent upon the presence of a particular structure (i.e., an antigenic determinant or epitope) of the protein that is recognized by the binding molecule.

The present invention provides novel GPCR polynucleotides and encoded GPCR polypeptides. The GPCRs according to this invention are preferably full-length molecules. More specifically, the GPCRs according to the invention are “class A” GPCRs. Class A GPCRs are rhodopsin-like GPCRs and they constitute the largest sub-class of the GPCR superfamily. Class A GPCRs are comprised of, but not limited to, amine, peptide, hormone protein, rhodopsin, olfactory, prostanoid, nucleotide-like, cannabis, platelet activating factor, gonadotropin-releasing hormone, TRH-Secretagogue, melatonin, viral, lysosphingolipid, and many orphan GPCRs.

GPCRs can also include dopamine receptors, rhodopsin receptors, kinin receptors, N-formyl peptide receptors, opioid receptors, calcitonin receptors, adrenergic receptors, endothelin receptors, cAMP receptors, adenosine receptors, muscarinic receptors, acetylcholine receptors, serotonin receptors, histamine receptors, thrombin receptors, follicle stimulating hormone receptors, opsin receptors, endothelial differentiation gene-1 receptors, odorant receptors, or cytomegalovirus receptors.

GPCR polynucleotides and/or polypeptides are useful for diagnosing diseases related to over- or under-expression of GPCR proteins. For example, such GPCR-associated diseases can be assessed by identifying mutations in a GPCR gene using GPCR probes or primers, or by determining GPCR protein or mRNA expression levels. GPCR polypeptides are also useful for screening compounds which affect activity of the protein. The invention further encompasses the polynucleotides encoding the GPCR polypeptides and the use of the GPCR polynucleotides or polypeptides, or compositions thereof, in the screening, diagnosis, treatment, or prevention of disorders associated with aberrant or uncontrolled cellular growth and/or function, such as neoplastic diseases (for example, cancers and tumors).

GPCR probes or primers can be used, for example, to screen for diseases associated with GPCRs. The primers of the invention are determined from the disclosed GPCR nucleic acid sequences.

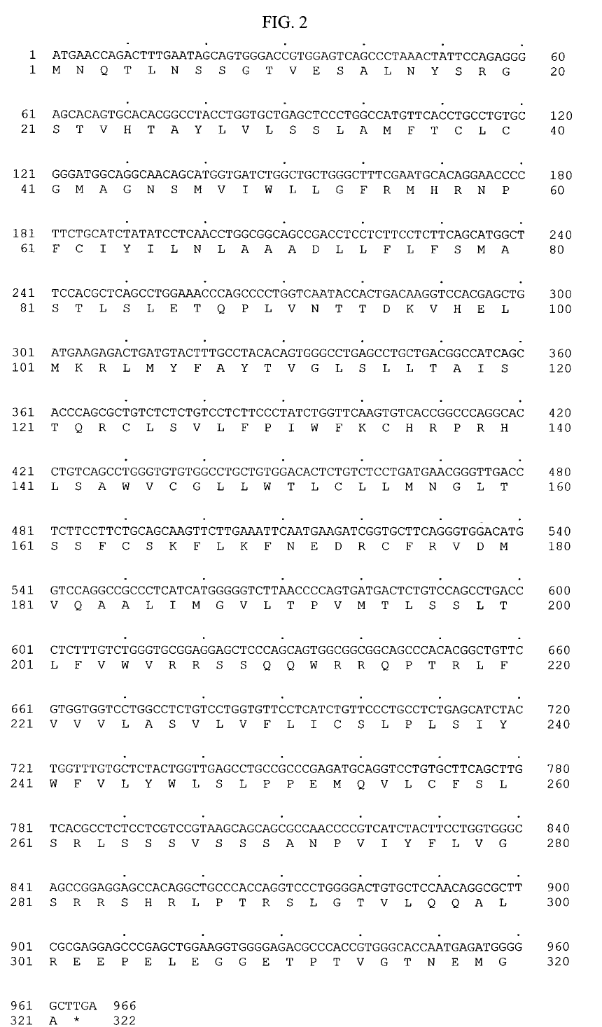

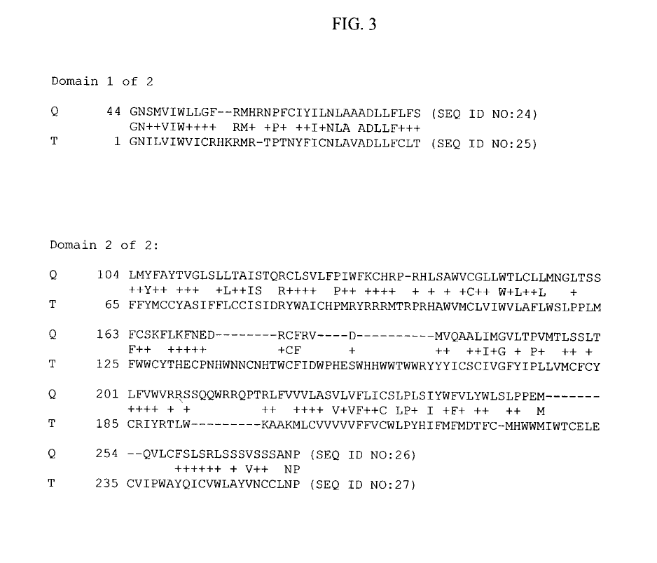

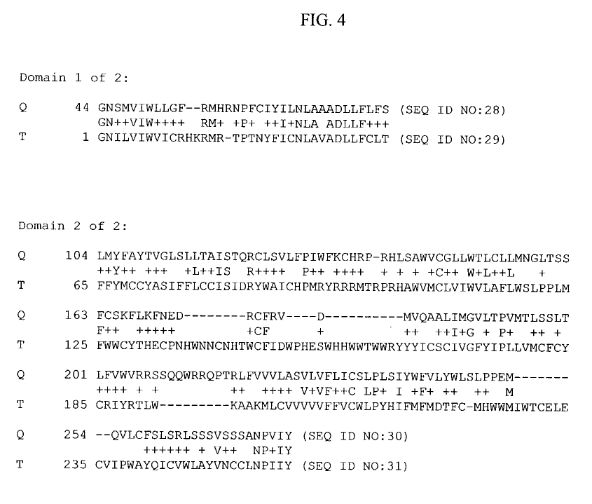

In one of its embodiments, the present invention encompasses a polypeptide comprising the amino acid sequence of SEQ ID NO:2 as shown in FIG. 2. The HGPRBMY31 polypeptide is 307 amino acids in length (MW=34.41 Kd) and shares amino acid sequence homology with “class A” GPCRs. In FIGS. 10A-B, the HGPRBMY31 polypeptide (SEQ ID NO:2) shares percent identity and percent similarity with GPCRs, wherein “similar” amino acids are those which have the same/similar physical properties and in many cases, the function is conserved with similar residues. For example, amino acids Lysine and Arginine are similar; whereas residues such as Proline and Cysteine do not share any physical property and they are not considered similar. The HGPRBMY31 polypeptide shares 30.85% identity and 37.967% similarity with the amino acid sequence of Cavia porcellus C5A anaphylatoxin chemotactic receptor (SEQ ID NO:7; C5AR_CAVPO; SWISS-PROT Acc. No.:070129); shares 33.45% sequence identity and 41.3% similarity with the orangatan N-formyl peptide receptor-like 2 receptor fragment (SEQ ID NO:8; FML2_PONPY; SWISS-PROT Acc. No.:P79237); shares 33% identity and 43.23% similarity with the mouse GPCR, GPR90 (SEQ ID NO:9; GPR90_MOUSE; SWISS-PROT Acc. No.:P2_{—}120389); shares 99.67% identity and 99.67% sequence similarity with the HGPRBMY31 variant (SEQ ID NO:4; HGPRBMY31_VARIANT); shares 34.9% sequence identity and 46.64% similarity with the human Mas proto-oncogene (SEQ ID NO:10; MAS_HUMAN; SWISS-PROT Acc. No.:P04201); shares 35.24% identity and 45.64% similarity with the mouse Mas proto-oncogene (SEQ ID NO:11; MAS_MOUSE; SWISS-PROT Ace. No.: P30554, 035944); shares 36.24% identity and 48.32% sequence similarity with the rat Mas proto-oncogene (SEQ ID NO:12; MAS_RAT; SWISS-PROT Ace. No.: P12526); shares 33.9% identity and 44.86% sequence similarity with the human Mas related GPCR, MRG (SEQ ID NO:13; MRG_HUMAN; SWISS-PROT Acc. No.:P35410); shares 36.07% sequence identity and 43.61% similarity with the probable rat GPCR, RTA (SEQ ID NO:14; RTA_RAT; SWISS-PROT Ace. No.:P23749); and shares 35.91% sequence identity and 46.64% similarity with the mouse putative G protein coupled receptor (SEQ ID NO:15; ORPHAN_MOUSE; SWISS-PROT Ace. No.:P2_{—}137806). The top matching GPCR to HGPRBMY31 by sequence analysis using the BLAST program is the mouse G-Protein Coupled receptor, GPR90 (Genbank Accession No.: NP_{—}109651).

The Cavia porcellus C5A anaphylatoxin chemotactic receptor (SEQ ID NO:7; C5AR_CAVPO; SWISS-PROT Acc. No.:070129) is a G-protein coupled receptor for the chemotactic and inflammatory peptide anaphylatoxin C5A. This receptor stimulates chemotaxis, granule enzyme release, and superoxide anion production (Int. Immunol. 10:275-283(1998)).

The N-formyl peptide receptor-like 2 receptor fragment (SEQ ID NO:8; FML2_PONPY; SWISS-PROT Ace. No.:P79237) is a G-protein coupled receptor that represents low affinity receptor for N-formyl-methionyl peptides, which are powerful neutrophil chemotactic factors. Binding of FMLP to this receptor causes activation of neutrophils, which is mediated via a G-protein that activates a phosphatidylinositol-calcium second messenger system (IMMUNOGENETICS 44:446-452(1996)).

Variants of GPCR polypeptides are also encompassed by the present invention. Preferably, a GPCR variant has at least 75 to 80%, more preferably at least 85 to 90%, and even more preferably at least 90% amino acid sequence identity to a GPCR amino acid sequence disclosed herein, and more preferably, retains at least one biological, immunological, or other functional characteristic or activity of the non-variant GPCR polypeptide. Most preferred are GPCR variants or substantially purified fragments thereof having at least 95% amino acid sequence identity to those of SEQ ID NO:2. Variants of GPCR polypeptides or substantially purified fragments of the polypeptides can also include amino acid sequences that differ from the amino acid sequence of SEQ ID NO:2 only by conservative substitutions. The invention also encompasses polypeptide homologues of the amino acid sequence as set forth in SEQ ID NO:4. Preferably, the GPCR variant of HGPRBMY31 is HGPRBMY31_variant, having an nucleic acid sequence of SEQ ID NO:3 and amino acid sequence of SEQ ID NO:4.

The HGPRBMY31 variant polypeptide is 321 amino acids in length (36.117 Kd) and shares amino acid sequence homology with “class A” GPCRs, in particular HGPRBMY31. The HGPRBMY31 variant (SEQ ID NO:4) shares 31.72% identity and 38.19% similarity with the amino acid sequence of Cavia porcellus C5A anaphylatoxin chemotactic receptor (SEQ ID NO:7; C5AR_CAVPO; SWISS-PROT Acc. No.:O70129); 33.55% sequence identity and 41.69% similarity with the orangatan N-formyl peptide receptor-like 2 receptor fragment (SEQ ID NO:8; FML2_PONPY; SWISS-PROT Acc. No.:P79237); 34.4% identity and 44.59% similarity with the mouse GPCR, GPR90 (SEQ ID NO:9; GPR90MOUSE; SWISS-PROT Acc. No.:P2_{—}120389); 99.67% identity and 99.67% sequence similarity with the HGPRBMY31 (SEQ ID NO:2; HGPRBMY31); 35.69% sequence identity and 47.27% similarity with the human Mas proto-oncogene (SEQ ID NO:10; MAS_HUMAN; SWISS-PROT Acc. No.:P04201); 36.01% identity and 46.3% similarity with the mouse Mas proto-oncogene (SEQ ID NO:11; MAS_MOUSE; SWISS-PROT Acc. No.: P30554, 035944); 36.98% identity and 48.88% sequence similarity with the rat Mas proto-oncogene (SEQ ID NO:12; MAS_RAT; SWISS-PROT Acc. No.: P12526); 36.07% identity and 46.56% sequence similarity with the human Mas related GPCR, MRG (SEQ ID NO:13; MRG_HUMAN; SWISS-PROT Acc. No.:P35410); 37.34% sequence identity and 45.25% similarity with the probable rat GPCR, RTA (SEQ ID NO:14; RTA_RAT; SWISS-PROT Ace. No.:P23749); and 36.66% sequence identity and 47.27% similarity with the mouse putative G protein coupled receptor (SEQ ID NO:15; ORPHAN_MOUSE; SWISS-PROT Ace. No.:P2_{—}137806).

The GAP global alignment program in GCG Sequence was used to calculate the percent identity and similarity values as compared with other sequences in the alignments of FIGS. 10A-10B. The following parameters were used in the GAP program: gap creation penalty=8; gap extension penalty=2. This program uses an algorithm based on a reference by Needleman, S. and Wunsch, C. (J. Mol. Biol. 48(3):443-53, 1970).

In another embodiment, the present invention encompasses polynucleotides which encode GPCR polypeptides. Accordingly, any nucleic acid sequence that encodes the amino acid sequence of a GPCR polypeptide of the invention can be used to produce recombinant molecules that express a GPCR protein. More particularly, the invention encompasses the GPCR polynucleotides comprising the nucleic acid sequences of SEQ ID NO:1 and 3. The present invention also provides GPCR cDNA clones, deposited at the American Type Culture Collection (ATCC), 10801 University Boulevard, Manassas, Va. 20110-2209 on Dec. 22, 2001 and under ATCC Accession No(s). PTA-3949 according to the terms of the Budapest Treaty.

The HGPRBMY31 polypeptide was predicted to comprise seven transmembrane domains (TM1 to TM7) located from about amino acid 28 to about amino acid 49 (TM1); from about amino acid 61 to about amino acid 79 (TM2); from about amino acid 105 to about amino acid 127 (TM3); from about amino acid 141 to about amino acid 164 (TM4); from about amino acid 186 to about amino acid 205 (TM5); from about amino acid 219 to about amino acid 242 (TM6); and/or from about amino acid 255 to about amino acid 278 (TM7) of SEQ ID NO:2 (FIGS. 1A-D). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

The present invention also encompasses the polypeptide sequences that intervene between each of the predicted HGPRBMY31 transmembrane domains. Since these regions are solvent accessible either extracellularly or intracellularly, they are particularly useful for designing antibodies specific to each region. Such antibodies may be useful as antagonists or agonists of the HGPRBMY31 full-length polypeptide and may modulate its activity.

The present invention encompasses polynucleotides corresponding to the full-length encoding sequence of the HGPRBMY31 polypeptide. Specifically, the present invention encompasses polynucleotides 90 to 1010 of SEQ ID NO:1.

The present invention also encompasses polynucleotides corresponding to the full-length encoding sequence of the HGPRBMY31 polypeptide minus the start codon. Specifically, the present invention encompasses polynucleotides 93 to 1010 of SEQ ID NO:1.

The present invention encompasses polynucleotides corresponding to the full-length encoding sequence of the HGPRBMY31 variant polypeptide. Specifically, the present invention encompasses polynucleotides 1 to 963 of SEQ ID NO:3.

The present invention also encompasses polynucleotides corresponding to the full-length encoding sequence of the HGPRBMY31 variant polypeptide minus the start codon. Specifically, the present invention encompasses polynucleotides 4 to 963 of SEQ ID NO:3.

The HGPRBMY31 polypeptide has been shown to comprise four glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following asparagine glycosylation site polypeptides are encompassed by the present invention: MNQTLNSSGT (SEQ ID NO:32), MNQTLNSSGTVESA (SEQ ID NO:33), VESALNYSRGSTVH (SEQ ID NO:34), and/or TQPLVNTTDKVHEL (SEQ ID NO:35). Polynucleotides encoding these polypeptides are also provided. The present invention also encompasses the use of these HGPRBMY31 asparagine glycosylation site polypeptides as immunogenic and/or antigenic epitopes as described elsewhere herein.

The HGPRBMY31 polypeptides of the present invention were determined to comprise several phosphorylation sites based upon the Motif algorithm (Genetics Computer Group, Inc.). The phosphorylation of such sites may regulate some biological activity of the HGPRBMY31 polypeptide. For example, phosphorylation at specific sites may be involved in regulating the proteins ability to associate or bind to other molecules (e.g., proteins, ligands, substrates, DNA, etc.).

Specifically, the HGPRBMY31 polypeptide was predicted to comprise one cAMP- and cGMP-dependent protein kinase phosphorylation site using the Motif algorithm (Genetics Computer Group, Inc.). There has been a number of studies relative to the specificity of cAMP- and cGMP-dependent protein kinases. Both types of kinases appear to share a preference for the phosphorylation of serine or threonine residues found close to at least two consecutive N-terminal basic residues.

A consensus pattern for cAMP- and cGMP-dependent protein kinase phosphorylation sites is as follows: [RK](2)-x-[ST], wherein “x” represents any amino acid, and S or T is the phosphorylation site.

Additional information specific to cAMP- and cGMP-dependent protein kinase phosphorylation sites may be found in reference to the following publication: Fremisco J. R., Glass D. B., Krebs E. G, J. Biol. Chem. 255:4240-4245(1980); Glass D. B., Smith S. B., J. Biol. Chem. 258:14797-14803(1983); and Glass D. B., El-Maghrabi M. R., Pilkis S. J., J. Biol. Chem. 261:2987-2993(1986); which is hereby incorporated herein in its entirety.

In preferred embodiments, the following cAMP- and cGMP-dependent protein kinase phosphorylation site polypeptide is encompassed by the present invention: LFVWVRRSSQQWRR (SEQ ID NO:21). Polynucleotides encoding this polypeptide are also provided. The present invention also encompasses the use of this cAMP- and cGMP-dependent protein kinase phosphorylation site polypeptide as an immunogenic and/or antigenic epitope as described elsewhere herein.

The HGPRBMY31 polypeptide was predicted to comprise two PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: PLVNTTDKVHELM (SEQ ID NO:22), and/or LTAISTQRCLSVL (SEQ ID NO:23). Polynucleotides encoding these polypeptides are also provided. The present invention also encompasses the use of these HGPRBMY31 PKC phosphorylation site polypeptides as immunogenic and/or antigenic epitopes as described elsewhere herein.

The HGPRBMY31 polypeptide was predicted to comprise four N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: TLNSSGTVESALNYSR (SEQ ID NO:36), FTCLCGMAGNSMVIWL (SEQ ID NO:37), SAWVCGLLWTLCLLMN (SEQ ID NO:38), and/or CLLMNGLTSSFCSKFL (SEQ ID NO:39). Polynucleotides encoding these polypeptides are also provided. The present invention also encompasses the use of these N-myristoylation site polypeptides as immunogenic and/or antigenic epitopes as described elsewhere herein.

TABLE II

| | | ATCC | | | | | | | |
|---|---|---|---|---|---|---|---|---|---|
| | | DEPOSIT | | NT | Total NT | 5′ NT of | | AA | |
| Gene | CDNA | NO. Z | | SEQ ID. | Seq of | Start Codon | 3′ NT | Seq ID | Total AA |
| No. | CloneID | AND DATE | Vector | No. X | Clone | of ORF | of ORF | No. Y | of ORF |
| 1. | HGPRBMY31 | PTA-3949 | pSport1 | 1 | 3791 | 90 | 1010 | 2 | 307 |
| | (also referred | Dec. 12, 2001 | | | | | | | |
| | to as GPCR61) | | | | | | | | |
| 2. | HGPRBMY31 | N/A | pSport1 | 3 | 966 | 1 | 963 | 4 | 321 |
| | variant (also | | | | | | | | |
| | referred to as | | | | | | | | |
| | GPCR-151) | | | | | | | | |

Table II summarizes the information corresponding to each “Gene No.” described above. The nucleotide sequence identified as “NT SEQ ID NO:X” was assembled from partially homologous (“overlapping”) sequences obtained from the “cDNA clone ID” identified in Table II and, in some cases, from additional related DNA clones. The overlapping sequences were assembled into a single contiguous sequence of high redundancy (usually several overlapping sequences at each nucleotide position), resulting in a final sequence identified as SEQ ID NO:X. However, for the purposes of the present invention, SEQ ID NO:X may refer to any polynucleotide of the present invention.

The cDNA Clone ID was deposited on the date and given the corresponding deposit number listed in “ATCC Deposit No:Z and Date.” “Vector” refers to the type of vector contained in the cDNA Clone ID.

“Total NT Seq. Of Clone” refers to the total number of nucleotides in the clone contig identified by “Gene No.” The deposited clone may contain all or most of the sequence of SEQ ID NO:X. The nucleotide position of SEQ ID NO:X of the putative start codon (methionine) is identified as “5′ NT of Start Codon of ORF.”

The translated amino acid sequence, beginning with the methionine, is identified as “AA SEQ ID NO:Y” although other reading frames can also be easily translated using known molecular biology techniques. The polypeptides produced by these alternative open reading frames are specifically contemplated by the present invention.

The total number of amino acids within the open reading frame of SEQ ID NO:Y is identified as “Total AA of ORF”.

SEQ ID NO:X (where X may be any of the polynucleotide sequences disclosed in the sequence listing) and the translated SEQ ID NO:Y (where Y may be any of the polypeptide sequences disclosed in the sequence listing) are sufficiently accurate and otherwise suitable for a variety of uses well known in the art and described further herein. For instance, SEQ ID NO:X is useful for designing nucleic acid hybridization probes that will detect nucleic acid sequences contained in SEQ ID NO:X or the cDNA contained in the deposited clone. These probes will also hybridize to nucleic acid molecules in biological samples, thereby enabling a variety of forensic and diagnostic methods of the invention. Similarly, polypeptides identified from SEQ ID NO:Y may be used, for example, to generate antibodies which bind specifically to proteins containing the polypeptides and the proteins encoded by the cDNA clones identified in Table II.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides may cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1 and the predicted translated amino acid sequence identified as SEQ ID NO:2, but also a sample of plasmid DNA containing a cDNA of the invention deposited with the ATCC, as set forth in Table II. The nucleotide sequence of each deposited clone can readily be determined by sequencing the deposited clone in accordance with known methods. The predicted amino acid sequence can then be verified from such deposits. Moreover, the amino acid sequence of the protein encoded by a particular clone can also be directly determined by peptide sequencing or by expressing the protein in a suitable host cell containing the deposited cDNA, collecting the protein, and determining its sequence.

The present invention also relates to the genes corresponding to SEQ ID NO:1, SEQ ID NO:3, or the deposited clone. The corresponding gene can be isolated in accordance with known methods using the sequence information disclosed herein. Such methods include preparing probes or primers from the disclosed sequence and identifying or amplifying the corresponding gene from appropriate sources of genomic material.

As will be appreciated by the skilled practitioner in the art, the degeneracy of the genetic code results in many nucleotide sequences that can encode the described GPCR polypeptides. Some of the sequences bear minimal or no homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring GPCR, and all such variations are to be considered as being specifically disclosed and able to be understood by the skilled practitioner.

Although nucleic acid sequences which encode the GPCR polypeptides and variants thereof are preferably capable of hybridizing to the nucleotide sequence of the naturally occurring GPCR polypeptide under appropriately selected conditions of stringency, it may be advantageous to produce nucleotide sequences encoding GPCR polypeptides, or derivatives thereof, which possess a substantially different codon usage. For example, codons may be selected to increase the rate at which expression of the peptide/polypeptide occurs in a particular prokaryotic or eukaryotic host in accordance with the frequency with which particular codons are utilized by the host. Another reason for substantially altering the nucleotide sequence encoding a GPCR polypeptide, or its derivatives, without altering the encoded amino acid sequences, includes the production of RNA transcripts having more desirable properties, such as a greater half-life, than transcripts produced from the naturally occurring sequence.

The present invention also encompasses production of DNA sequences, or portions thereof, which encode the GPCR polypeptides, or derivatives thereof, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding a GPCR polypeptide, or any fragment thereof.

In an embodiment of the present invention, a gene delivery vector containing the polynucleotide, or functional fragment thereof is provided. Preferably, the gene delivery vector contains the polynucleotide, or functional fragment thereof comprising an isolated and purified polynucleotide encoding a human GPCR having the sequence as set forth in any one of SEQ ID NO:1 and 3.

It will also be appreciated by those skilled in the pertinent art that a longer oligonucleotide probe, or mixtures of probes, for example, degenerate probes, can be used to detect longer, or more complex, nucleic acid sequences, such as, for example, genomic or full length DNA. In such cases, the probe may comprise at least 20-300 nucleotides, preferably, at least 30-100 nucleotides, and more preferably, 50-100 nucleotides.

The present invention also provides methods of obtaining the full length sequence of the GPCR polypeptides as described herein. In one instance, the method of multiplex cloning was devised as a means of extending large numbers of bioinformatic gene predictions into full length sequences by multiplexing probes and cDNA libraries in an effort to minimize the overall effort typically required for cDNA cloning. The method relies on the conversion of plasmid-based, directionally cloned cDNA libraries into a population of pure, covalently-closed, circular, single-stranded molecules and long biotinylated DNA oligonucleotide probes designed from predicted gene sequences.

Probes and libraries were subjected to solution hybridization in a formamide buffer which has been found to be superior to aqueous buffers typically used in other biotin/streptavidin cDNA capture methods (i.e., GeneTrapper). The hybridization was performed without prior knowledge of the clones represented in the libraries. Hybridization was performed two times. After the first selection, the isolated sequences were screened with PCR primers specific for the targeted clones. The second hybridization was carried out with only those oligo probes whose gene-specific PCR assays gave positive results.

The secondary hybridization serves to ‘normalize’ the selected library, thereby decreasing the amount of screening needed to identify particular clones. The method is robust and sensitive. Typically, dozens of cDNAs are isolated for any one particular gene, thereby increasing the chances of obtaining a full length cDNA. The entire complexity of any cDNA library is screened in the solution hybridization process, which is advantageous for finding rare sequences. The procedure is scaleable, with 50 oligonucleotide probes per experiment currently being used, although this is not to be considered a limiting number.

Using bioinformatic predicted gene sequence, the following types of PCR primers and cloning oligos can be designed: A) PCR primer pairs that reside within a single predicted exon; B) PCR primer pairs that cross putative exon/intron boundaries; and C) 80mer antisense and sense oligos containing a biotin moiety on the 5′ end. The primer pairs of the A type above are optimized on human genomic DNA; the B type primer pairs are optimized on a mixture of first strand cDNAs made with and without reverse transcriptase. Primers will be optimized using mRNA derived from appropriate tissues sources, for example, brain, lung, uterus, cartilage, and testis poly A+RNA.

The information obtained with the B type primers is used to assess those putative expressed sequences which can be experimentally observed to have reverse transcriptase-dependent expression. The primer pairs of the A type are less stringent in terms of identifying expressed sequences. However, because they amplify genomic DNA as well as cDNA, their ability to amplify genomic DNA provides for the necessary positive control for the primer pair. Negative results with the B type are subject to the caveat that the sequence(s) may not be expressed in the tissue first strand that is under examination.

The biotinylated 80-mer oligonucleotides are added en mass to pools of single strand cDNA libraries. Up to 50 probes have been successfully used on pools for 15 different libraries. After the primary selection is performed, all of the captured DNA is repaired to double strand form using the T7 primer for the commercial libraries in pCMVSPORT, and the Sp6 primer for other constructed libraries in pSPORT. The resulting DNA is electroporated into E. coli DH12S and plated onto 150 mm plates with nylon filters. The cells are scraped and a frozen stock is made, thereby comprising the primary selected library.

One-fifth of the library is generally converted into single strand form and the DNA is assayed with gene specific primer pairs (GSPs). The next round of solution hybridization capture is carried out with 80 mer oligos for only those sequences that are positive with the gene-specific-primers. After the second round, the captured single strand DNAs are repaired with a pool of GSPs, where only the primer complementary to polarity of the single-stranded circular DNA is used (i.e., the antisense primer for pCMVSPORT and pSPORT1 and the sense primer for pSPORT2).

The resulting colonies are screened by PCR using the GSPs. Typically, greater than 80% of the clones are positive for any given GSP. The entire 96 well block of clones is subjected to “mini-prep”, as known in the art, and each of clones is sized by either PCR or restriction enzyme digestion. A selection of different sized clones for each targeted sequence is chosen for transposon-hopping and DNA sequencing.

Preferably, as for established cDNA cloning methods used by the skilled practitioner, the libraries employed are of high quality. High complexity and large average insert size are optimal. High Pressure Liquid Chromatography (HPLC) may be employed as a means of fractionating cDNA for the purpose of constructing libraries.

Another embodiment of the present invention provides a method of identifying full-length genes encoding the disclosed polypeptides. The GPCR polynucleotides of the present invention, the polynucleotides encoding the GPCR polypeptides of the present invention, or the polypeptides encoded by the deposited clone(s) preferably represent the complete coding region (i.e., full-length gene).

Several methods are known in the art for the identification of the 5′ or 3′ non-coding and/or coding portions of a given gene. The methods described herein are exemplary and should not be construed as limiting the scope of the invention. These methods include, but are not limited to, filter probing, clone enrichment using specific probes, and protocols similar or identical to 5′ and 3′ “RACE” protocols that are well known in the art. For instance, a method similar to 5′ RACE is available for generating the missing 5′ end of a desired full-length transcript. (Fromont-Racine et al., Nucleic Acids Res. 21(7):1683-1684 (1993)).

Briefly, in the RACE method, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full-length gene.

The above method utilizes total RNA isolated from the desired source, although poly-A+ RNA can be used. The RNA preparation is treated with phosphatase, if necessary, to eliminate 5′ phosphate groups on degraded or damaged RNA that may interfere with the later RNA ligase step. The phosphatase is preferably inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

The above-described modified RNA preparation is used as a template for first strand cDNA synthesis employing a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene. It may also be advantageous to optinuze the RACE protocol to increase the probability of isolating additional 5′ or 3′ coding or non-coding sequences. Various methods of optimizing a RACE protocol are known in the art; for example, a detailed description summarizing these methods can be found in B. C. Schaefer, Anal. Biochem., 227:255-273, (1995).

An alternative method for carrying out 5′ or 3′ RACE for the identification of coding or non-coding nucleic acid sequences is provided by Frohman, M. A., et al., Proc. Nat'l. Acad. Sci. USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation for an encoded product. A brief description of a modification of the original 5′ RACE procedure is as follows. Poly A+ or total RNA is reverse transcribed with Superscript II (Gibco/BRL) and an antisense or an I complementary primer specific to any one of the cDNA sequences provided as SEQ ID NO:1 and 3. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoIJ Sail and Clal) at the 5′ end and a primer containing just these restriction sites. This double-stranded cDNA is PCR amplified for 40 cycles with the same primers, as well as a nested cDNA-specific antisense primer. The PCR products are size-separated on an ethidium bromide-agarose gel and the region of gel containing cDNA products having the predicted size of missing protein-coding DNA is removed.

cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length genes. A second kit is available from Clontech which is a modification of a related technique, called single-stranded ligation to single-stranded cDNA, (SLIC), developed by Dumas et al., Nucleic Acids Res., 19:5227-32(1991). The major difference in the latter procedure is that the RNA is alkaline hydrolyzed after reverse transcription and RNA ligase is used to join a restriction site-containing anchor primer to the first-strand cDNA. This obviates the necessity for the dA-tailing reaction which results in a polyT stretch that can impede sequencing.

An alternative to generating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

Also encompassed by the present invention are polynucleotide sequences that are capable of hybridizing to the novel GPCR nucleic acid sequences, as set forth in SEQ ID NO:1 and 3, under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the GPCR sequences of SEQ ID NO:1 and 3, and other sequences which are degenerate to those which encode the novel GPCR polypeptides. For example, a non-limiting example of moderate stringency conditions include prewashing solution of 2×SSC, 0.5% SDS, 1.0 mM EDTA, pH 8.0, and hybridization conditions of 50° C., 5×SSC, overnight.

The nucleic acid sequence encoding the GPCR proteins of the present invention may be extended by utilizing a partial nucleotide sequence and employing various methods known in the art to detect upstream sequences such as promoters and regulatory elements. For example, one method that can be employed is restriction-site PCR, which utilizes universal primers to retrieve unknown sequence adjacent to a known locus (See, e.g., G. Sarkar, 1993, PCR Methods Applic., 2:318-322). In particular, genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences, Inc., Plymouth, Minn.), or another appropriate program, to be 22-30 nucleotides in length, to have a GC content of 50% or more, and to anneal to the target sequence at temperatures about 68° C.-72° C. The method uses several restriction enzymes to generate a suitable fragment in the known region of a gene. The fragment is then circularized by intramolecular ligation and used as a PCR template.

Another method which may be used to amplify or extend sequences is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. Bacterial artificial chromosomes (BACs) are also used for such applications. In addition, PCR, nested primers, and PROMOTERFINDER libraries can be used to “walk” genomic DNA (Clontech; Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are also preferable, since such libraries will contain more sequences that comprise the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and generally available in the art. The methods may employ such enzymes as the Klenow fragment of DNA polymerase I, SEQUENASE (US Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton; Reno, Nev.), Peltier Thermal Cycler (PTC200; MJ Research; Watertown, Mass.) and the ABI Catalyst and 373 and 377 DNA sequencers (PE Biosystems). Commercially available capillary electrophoresis systems may be used to analyze the size or confirm the nucleotide sequence of sequencing or PCR products. Capillary electrophoresis is especially preferable for the sequencing of small pieces of DNA, which might be present in limited amounts in a particular sample.

In another embodiment of the present invention, polynucleotide sequences or portions thereof which encode GPCR polypeptides or peptides can comprise recombinant DNA molecules to direct the expression of GPCR polypeptide products, peptide fragments, or functional equivalents thereof, in appropriate host cells. Because of the inherent degeneracy of the genetic code, other DNA sequences, which encode substantially the same or a functionally equivalent amino acid sequence, may be produced and these sequences may be used to clone and express the GPCR proteins as described.

As will be appreciated by those having skill in the art, it may be advantageous to produce GPCR polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequences of the present invention can be engineered using methods generally known in the art in order to alter the GPCR polypeptide-encoding sequences for a variety of reasons, including, but not limited to, alterations which modify the cloning, processing, and/or expression of the gene product. DNA shuffling by random fragmentation, PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and the like.

In another embodiment of the present invention, natural, modified, or recombinant nucleic acid sequences encoding the GPCR polypeptides may be ligated to a heterologous sequence to encode a fusion (or chimeric or hybrid) protein. For example, a fusion protein can comprise any one of the amino acid sequences as set forth in SEQ ID NO:2 and 4, and an amino acid sequence of an Fc portion (or constant region) of a human immunoglobulin protein. The fusion protein may further comprise an amino acid sequence that differs from any one of SEQ ID NO:2 and 4 only by conservative substitutions. As another example, to screen peptide libraries for inhibitors of GPCR activity, it may be useful to encode a chimeric GPCR protein that can be recognized by a commercially available antibody. A fusion protein may also be engineered to contain a cleavage site located between the GPCR protein-encoding sequence and the heterologous protein sequence, so that the GPCR protein may be cleaved and purified away from the heterologous moiety.

In a further embodiment, sequences encoding the GPCR polypeptides may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the GPCR protein itself, or a fragment or portion thereof, may be produced using chemical methods to synthesize the amino acid sequence of the GPCR polypeptide, or a fragment or portion thereof. For example, peptide synthesis can be performed using various solid-phase techniques (J. Y. Roberge et al., 1995, Science, 269:202-204) and automated synthesis can be achieved, for example, using the ABI 431A Peptide Synthesizer (PE Biosystems).

The newly synthesized GPCR polypeptide or peptide can be substantially purified by preparative high performance liquid chromatography (e.g., T. Creighton, 1983, Proteins, Structures and Molecular Principles, W. H. Freeman and Co., New York, N.Y.), by reverse-phase high performance liquid chromatography (HPLC), or other purification methods as known and practiced in the art. The composition of the synthetic peptides may be confirmed by amino acid analysis or sequencing (e.g., the Edman degradation procedure; Creighton, supra). In addition, the amino acid sequence of a GPCR polypeptide, or any portion thereof, can be altered during direct synthesis and/or combined using chemical methods with sequences from other proteins, or any part thereof, to produce a variant polypeptide.

To express a biologically active GPCR polypeptide or peptide, the nucleotide sequences encoding the GPCR polypeptide, or functional equivalents, may be inserted into an appropriate expression vector, i.e., a vector, which contains the necessary elements for the transcription and translation of the inserted coding sequence.

In one embodiment of the present invention, an expression vector contains an isolated and purified polynucleotide sequence as set forth in any one of SEQ ID NO:1 and 3, encoding a human GPCR, or a functional fragment thereof, in which the human GPCR comprises the amino acid sequence as set forth in any one of SEQ ID NO:2 and 4. Alternatively, an expression vector can contain the complement of the aforementioned GPCR nucleic acid sequences.

Expression vectors derived from retroviruses, adenovirus, herpes or vaccinia viruses, or from various bacterial plasmids can be used for the delivery of nucleotide sequences to a target organ, tissue or cell population. Methods, which are well known to those skilled in the art, may be used to construct expression vectors containing sequences encoding one or more GPCR polypeptide along with appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding the GPCR polypeptides or peptides. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems, including mammalian cell systems. The host cell employed is not limiting to the present invention. Preferably, the host cell of the invention contains an expression vector comprising an isolated and purified polynucleotide having a nucleic acid sequence selected from any one of SEQ ID NO:1 and 3, and encoding a human GPCR of this invention, or a functional fragment thereof, comprising an amino acid sequence as set forth in any one of SEQ ID NO:2 and 4.

Bacterial artificial chromosomes (BACs) may be used to deliver larger fragments of DNA than can be contained and expressed in a plasmid vector. BACs are vectors used to clone DNA sequences of 100-300 kb, on average 150 kb, in size in E. coli cells. BACs are constructed and delivered via conventional delivery methods (e.g., liposomes, polycationic amino polymers, or vesicles) for therapeutic purposes.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. Specific initiation signals may also be used to achieve more efficient translation of sequences encoding a GPCR polypeptide. Such signals include the ATG initiation codon and adjacent sequences. In cases where sequences encoding a GPCR polypeptide, its initiation codon, and upstream sequences are inserted into the appropriate expression vector, no additional transcriptional or translational control signals may be needed. However, in cases where only a GPCR coding sequence, or a fragment thereof, is inserted, exogenous translational control signals, including the ATG initiation codon, are optimally provided. Furthermore, the initiation codon should be in the correct reading frame to insure translation of the entire insert. Exogenous translational elements and initiation codons can be of various origins, both natural and synthetic. The efficiency of expression can be enhanced by the inclusion of enhancers which are appropriate for the particular cell system that is used, such as those described in the literature (see, e.g., D. Scharf et al., 1994, Results Probl. Cell Differ., 20:125-162).

In bacterial systems, a number of expression vectors may be selected, depending upon the use intended for the expressed GPCR product. For example, when large quantities of expressed protein are needed for the generation of antibodies, vectors that direct high level expression of fusion proteins that can be readily purified may be used. Such vectors include, but are not limited to, the multifunctional E. coli cloning and expression vectors such as BLUESCRIPT (Stratagene), in which the sequence encoding the GPCR polypeptide can be ligated into the vector in-frame with sequences for the amino-terminal Met and the subsequent 7 residues of β-galactosidase, so that a hybrid protein is produced; pIN vectors (see, G. Van Heeke and S. M. Schuster, 1989, J. Biol. Chem., 264:5503-5509); and the like. pGEX vectors (Promega, Madison, Wis.) can also be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. Proteins made in such systems can be designed to include heparin, thrombin, or factor XA protease cleavage sites so that the cloned polypeptide of interest can be released from the GST moiety at will.

In mammalian host cells, a number of viral-based expression systems can be utilized. In cases where an adenovirus is used as an expression vector, sequences encoding the GPCR polypeptide may be ligated into an adenovirus transcription/translation complex containing the late promoter and tripartite leader sequence. Insertion into a non-essential E1 or E3 region of the viral genome may be used to obtain a viable virus which is capable of expressing GPCR polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells. Other expression systems can also be used, such as, but not limited to yeast, plant, and insect vectors.

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), 10801 University Boulevard, Manassas, Va. 20110-2209, and may be chosen to ensure the correct modification and processing of the foreign protein.

Host cells transformed with nucleotide sequences encoding a GPCR protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode a GPCR protein can be designed to contain signal sequences which direct secretion of the GPCR protein through a prokaryotic or eukaryotic cell membrane. Other constructions can be used to join nucleic acid sequences encoding a GPCR protein to a nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and GPCR protein may be used to facilitate purification. One such expression vector provides for expression of a fusion protein containing GPCR and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif, 3:263-281, while the enterokinase cleavage site provides a means for purifying from the fusion protein. For a discussion of suitable vectors for fusion protein production, see D. J. Kroll et al., 1993; DNA Cell Biol., 12:441-453.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk- or aprt-cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S.C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if the nucleic acid sequence encoding a GPCR polypeptide is inserted within a marker gene sequence, recombinant cells containing polynucleotide sequence encoding the GPCR polypeptide can be identified by the absence of marker gene function. Alternatively, a marker gene can be placed in tandem with a sequence encoding a GPCR polypeptide under the control of a single promoter. Expression of the marker gene in response to induction or selection typically indicates co-expression of the tandem gene.

A wide variety of labels and conjugation techniques are known and employed by those skilled in the art and may be used in various nucleic acid and amino acid assays. Means for producing labeled hybridization or PCR probes for detecting sequences related to polynucleotides encoding a GPCR polypeptide include oligo-labeling, nick translation, end-labeling, or PCR amplification using a labeled nucleotide. Alternatively, the sequences encoding a GPCR polypeptide of this invention, or any portion or fragment thereof, can be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which can be used include radionucleotides, enzymes, fluorescent, chemiluminescent, or chromogenic agents, as well as substrates, cofactors, inhibitors, magnetic particles, and the like.

Alternatively, host cells which contain the nucleic acid sequence coding for a GPCR polypeptide of the invention and which express the GPCR polypeptide product may be identified by a variety of procedures known to those having skill in the art. These procedures include, but are not limited to, DNA-DNA or DNA-RNA hybridizations and protein bioassay or immunoassay techniques, including membrane, solution, bead, microarray, or chip based technologies, for the detection and/or quantification of nucleic acid or protein.

The presence of polynucleotide sequences encoding GPCR polypeptides can be detected by DNA-DNA or DNA-RNA hybridization, or by amplification using probes, portions, or fragments of polynucleotides encoding a GPCR polypeptide. Nucleic acid amplification based assays involve the use of oligonucleotides or oligomers based on the nucleic acid sequences encoding a GPCR polypeptide to detect transformants containing DNA or RNA encoding GPCR polypeptide.

In addition to recombinant production, fragments of GPCR polypeptides may be produced by direct peptide synthesis using solid phase techniques (J. Merrifield, 1963, J. Am. Chem. Soc., 85:2149-2154). Protein synthesis may be performed using manual techniques or by automation. Automated synthesis may be achieved, for example, using ABI 431A Peptide Synthesizer (PE Biosystems). Various fragments of the GPCR polypeptides can be chemically synthesized separately and then combined using chemical methods to produce the full length molecule.

### Diagnostic Assays

In another embodiment of the present invention, antibodies which specifically bind to a GPCR polypeptide may be used for the diagnosis of conditions or diseases characterized by expression (or overexpression) of the GPCR polynucleotide or polypeptide, or in assays to monitor patients being treated with one or more of the GPCR polypeptides, or agonists, antagonists, or inhibitors of the novel GPCRs. The antibodies useful for diagnostic purposes can be prepared in the same manner as those described herein for use in therapeutic methods. Diagnostic assays for the GPCR polypeptides include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules known to those in the art may be used, several of which are described herein.

Another embodiment of the present invention contemplates a method of detecting a GPCR homologue, or an antibody-reactive fragment thereof, in a sample. The method comprises a) contacting the sample with an antibody specific for a GPCR polypeptide of the present invention, or an antigenic fragment thereof, under conditions in which an antigen-antibody complex can form between the antibody and the polypeptide or antigenic fragment thereof in the sample; and b) detecting the antigen-antibody complex formed in step a), wherein detection of the complex indicates the presence of the GPCR polypeptide, or an antigenic fragment thereof, in the sample.

Several assay protocols including enzyme-linked immunosorbent assay (ELISA), radioimmunoassay (RIA), and fluorescence activated cell sorting (FACS) for measuring GPCR polypeptide are known in the art and provide a basis for diagnosing altered or abnormal levels of GPCR polypeptide expression. Normal or standard values for GPCR polypeptide expression are established by combining body fluids or cell extracts taken from normal mammalian subjects, preferably human, with antibody to the GPCR polypeptide under conditions suitable for complex formation. The amount of standard complex formation may be quantified by various methods; photometric means are preferred. Quantities of GPCR polypeptide expressed in a subject or test sample, control sample, and disease samples from biopsied tissues are compared with the standard values. Deviation between standard and subject values establishes the parameters for diagnosing disease.

A variety of protocols for detecting and measuring the expression of GPCR polypeptide using either polyclonal or monoclonal antibodies specific for the polypeptide, or epitopic portions thereof, are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on a GPCR polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216).

In another embodiment of the present invention, a method of using a GPCR-encoding polynucleotide sequence to purify a molecule or compound in a sample, wherein the molecule or compound specifically binds to the polynucleotide, is contemplated. The method comprises: a) combining a GPCR-encoding polynucleotide of the invention with a sample undergoing testing to determine if the sample contains the molecule or compound, under conditions to allow specific binding; b) detecting specific binding between the GPCR-encoding polynucleotide and the molecule or compound, if present; c) recovering the bound polynucleotide; and d) separating the polynucleotide from the molecule or compound, thereby obtaining a purified molecule or compound.

This invention also relates to a method of using GPCR polynucleotides as diagnostic reagents. For example, the detection of a mutated form of the GPCR gene associated with a dysfunction can provide a diagnostic tool that can add to or define diagnosis of a disease or susceptibility to a disease which results from under-expression, over-expression, or altered expression of GPCRs. Individuals carrying mutations in the GPCR gene may be detected at the DNA level by a variety of techniques.

Nucleic acids for diagnosis may be obtained from various sources of a subject, for example, from cells, tissue, blood, urine, saliva, tissue biopsy or autopsy material. Genomic DNA may be used directly for detection or may be amplified by using PCR or other amplification techniques prior to analysis. RNA or cDNA may also be used in similar fashion. Deletions and insertions in GPCR-encoding polynucleotide can be detected by a change in size of the amplified product compared with that of the normal genotype. Hybridizing amplified DNA to labeled GPCR polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, for example, Myers et al., Science (1985) 230:1242. Sequence changes at specific locations may also be revealed by nuclease protection assays, such as RNase and S1 protection or the chemical cleavage method. (See Cotton et al., Proc. Natl. Acad. Sci., USA (1985) 85:43297-4401).

In another embodiment, an array of oligonucleotide probes comprising GPCR nucleotide sequence or fragments thereof can be constructed to conduct efficient screening of, for example, genetic mutations. Array technology methods are well known, have general applicability and can be used to address a variety of questions in molecular genetics, including gene expression, genetic linkage, and genetic variability (see for example: M. Chee et al., Science, 274:610-613, 1996).

Yet another aspect of the present invention involves a method of screening a library of molecules or compounds with a GPCR-encoding polynucleotide to identify at least one molecule or compound therein which specifically binds to the GPCR polynucleotide sequence. Such a method includes a) combining a GPCR-encoding polynucleotide of the present invention with a library of molecules or compounds under conditions to allow specific binding; and b) detecting specific binding, thereby identifying a molecule or compound, which specifically binds to a GPCR-encoding polynucleotide sequence, wherein the library is selected from DNA molecules, RNA molecules, artificial chromosome constructions, PNAs, peptides and proteins.

The present invention provides diagnostic assays for determining or monitoring through detection of a mutation in a GPCR gene (polynucleotide) described herein susceptibility to the following conditions, diseases, or disorders: cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, headache, migraine, schizophrenia, manic depression, delirium, dementia, severe mental retardation and dyskinesias, such as Huntington's disease or Gilles de la Tourette's syndrome.

In addition, such diseases, disorder, or conditions, can be diagnosed by methods of determining from a sample derived from a subject having an abnormally decreased or increased level of GPCR polypeptide or GPCR mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as a GPCR in a sample derived from a host are well known to those of skill in the art. Such assay methods include, without limitation, radioimmunoassays, competitive-binding assays, Western Blot analysis and ELISA assays.

In another of its aspects, this invention relates to a kit for detecting and diagnosing a GPCR-associated disease or susceptibility to such a disease, which comprises a GPCR polynucleotide, preferably the nucleotide sequence of SEQ ID NO: 1 and 3, or a fragment thereof; or a nucleotide sequence complementary to the GPCR polynucleotide of SEQ ID NO:1 and 3; or a GPCR polypeptide, preferably the polypeptide of SEQ ID NO:2 and 4, or a fragment thereof; or an antibody to the GPCR polypeptide, preferably to the polypeptide of SEQ ID NO:2 and 4, an epitope-containing portion thereof, or combinations of the foregoing. It will be appreciated that in any such kit, any of the previously mentioned components may comprise a substantial component. Also preferably included are instructions for use.

The GPCR polynucleotides which may be used in the diagnostic assays according to the present invention include oligonucleotide sequences, complementary RNA and DNA molecules, and PNAs. The polynucleotides may be used to detect and quantify GPCR-encoding nucleic acid expression in biopsied tissues in which expression (or under- or over-expression) of the GPCR polynucleotide may be determined, as well as correlated with disease. The diagnostic assays may be used to distinguish between the absence of GPCR, the presence of GPCR, or the excess expression of GPCR, and to monitor the regulation of GPCR polynucleotide levels during therapeutic treatment or intervention.

In a related aspect, hybridization with PCR probes which are capable of detecting polynucleotide sequences, including genomic sequences, encoding a GPCR polypeptide according to the present invention, or closely related molecules, may be used to identify nucleic acid sequences which encode a GPCR polypeptide. The specificity of the probe, whether it is made from a highly specific region, for example, about 8 to 10 contiguous nucleotides in the 5′ regulatory region, or a less specific region, for example, especially in the 3′ coding region, and the stringency of the hybridization or amplification (maximal, high, intermediate, or low) will determine whether the probe identifies only naturally occurring sequences encoding GPCR polypeptide, alleles thereof, or related sequences.

Probes may also be used for the detection of related sequences, and should preferably contain at least 50% of the nucleotides encoding the GPCR polypeptide. The hybridization probes or primers of this invention may be DNA or RNA and may be derived from the nucleotide sequences of SEQ ID NO:1 and 3, or may be derived from genomic sequence, including promoter, enhancer elements, and introns of the naturally occurring GPCR protein, wherein the probes or primers comprise a polynucleotide sequence capable of hybridizing with a polynucleotide of SEQ ID NO:1 and 3, under low, moderate, or high stringency conditions.

Methods for producing specific hybridization probes for DNA encoding the GPCR polypeptides include the cloning of a nucleic acid sequence that encodes the GPCR polypeptide, or GPCR derivatives, into vectors for the production of mRNA probes. Such vectors are known in the art, or are commercially available, and may be used to synthesize RNA probes in vitro by means of the addition of the appropriate RNA polymerases and the appropriate labeled nucleotides. Hybridization probes may be labeled by a variety of detector/reporter groups, including, but not limited to, radionucleotides such as ³²P or ³⁵S, or enzymatic labels, such as alkaline phosphatase coupled to the probe via avidin/biotin coupling systems, and the like.

The polynucleotide sequences encoding the GPCR polypeptides of this invention, or fragments thereof, may be used for the diagnosis of disorders associated with expression of GPCRs. The polynucleotide sequence encoding the GPCR polypeptide may be used in Southern or Northern analysis, dot blot, or other membrane-based technologies; in PCR technologies; or in dipstick, pin, ELISA or chip assays utilizing fluids or tissues from patient biopsies to detect the status of, for example, levels of, or overexpression of, a GPCR, or to detect altered GPCR expression or levels. Such qualitative or quantitative methods are commonly practiced in the art. In one embodiment, HGPRBMY31 polypeptides and polynucleotides, and variants thereof, preferably the HGPRBMY31 variant, are useful for diagnosing diseases related to over- or under-expression of HGPRBMY31 and HGPRBMY31 variant proteins. Briefly, mutations in the HGPRBMY31 and HGPRBMY31 variant genes are identified by using probes directed to HGPRMBY31 and HGPRBMY31 variant, determining proteins or mRNA expression levels to HGPRBMY31 and HGPRBMY31 variant.

In a particular aspect, a nucleotide sequence encoding a GPCR polypeptide as described herein may be useful in assays that detect activation or induction of various neoplasms, cancers, or other GPCR-related diseases, disorders, or conditions. The nucleotide sequence encoding a GPCR polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the GPCR polypeptide in the sample indicates the presence of the associated disease. Such assays may also be used to evaluate the efficacy of a particular therapeutic treatment regimen in animal studies, in clinical trials, or in monitoring the treatment or responsiveness of an individual patient.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to tumors or cancer, the presence of an abnormal amount or level of a GPCR transcript in biopsied tissue from an individual may indicate a predisposition for the development of the disease, or may provide a means for detecting the disease prior to the appearance of actual clinical symptoms. A more definitive diagnosis of this type may allow health practitioners to employ preventative measures or aggressive treatment earlier, thereby preventing the development or further progression of the tumor or cancer.

Additional diagnostic uses for oligonucleotides designed from the nucleic acid sequences encoding the novel GPCR polypeptides of this invention can involve the use of PCR. Such oligomers may be chemically synthesized, generated enzymatically, or produced from a recombinant source. Oligomers will preferably comprise two nucleotide sequences: one with sense orientation (5′→3′) and another with antisense orientation (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of GPCR include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or colorimetric response gives rapid quantification.

In one embodiment of the invention, a compound to be tested can be radioactively, colorimetrically or fluorimetrically labeled using methods well known in the art and incubated with the GPCR for testing. After incubation, it is determined whether the test compound is bound to the GPCR polypeptide. If so, the compound is to be considered a potential agonist or antagonist. Functional assays are performed to determine whether the receptor activity is activated (or enhanced or increased) or inhibited (or decreased or reduced). These assays include, but are not limited to, cell cycle analysis and in vivo tumor formation assays. Responses can also be measured in cells expressing the receptor using signal transduction systems including, but not limited to, protein phosphorylation, adenylate cyclase activity, phosphoinositide hydrolysis, guanylate cyclase activity, ion fluxes (i.e. calcium) and pH changes. These types of responses can either be present in the host cell or introduced into the host cell along with the receptor.

The present invention further embraces a method of screening for candidate compounds capable of modulating the activity of a GPCR-encoding polypeptide. Such a method comprises a) contacting a test compound with a cell or tissue expressing a GPCR polypeptide of the invention (e.g., recombinant expression); and b) selecting as candidate modulating compounds those test compounds that modulate activity of the GPCR polypeptide. Those candidate compounds which modulate GPCR activity are preferably agonists or antagonists, more preferably antagonists of GPCR activity.

### Therapeutic Assays

The GPCR proteins according to this invention may play a role in cell signaling, in cell cycle regulation, and/or in immune-related disorders. The GPCR proteins may further be involved in neoplastic, cardiovascular, and neurological disorders.

In one embodiment in accordance with the present invention, the novel GPCR protein may play a role in neoplastic disorders. An antagonist or inhibitor of the GPCR protein may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to GPCR may be used directly as an antagonist or indirectly as a targeting or delivery mechanism for bringing a pharmaceutical agent to cells or tissue which express the GPCR polypeptide.

In yet another embodiment of the present invention, an antagonist or inhibitory agent of the GPCR polypeptide may be administered therapeutically to an individual to prevent or treat an immunological disorder. Such disorders may include, but are not limited to, AIDS, HIV infection, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections, trauma, and neurological disorders including, but not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

In a preferred embodiment, HGPRBMY31 and the HGPRBMY31 variant can be used to treat diseases, disorders, and/or conditions related to acute heart failure, hypotension, hypertension, myocardial infarction, angina pectoris, endocrinal diseases, growth disorders, obesity, anorexia, bulimia, asthma, HIV infections, osteoporosis, cancers, neuropathic pain, Parkinson's disease, and cardiovascular, metabolic, psychotic, and neurological disorders. In addition, compounds acting on the HGPRBMY31 receptor, or variant receptor thereof, can be used as taste modifiers.

A preferred method of treating a GPCR associated disease, disorder, syndrome, or condition in a mammal comprises administration of a modulator, preferably an inhibitor or antagonist, of a GPCR polypeptide or homologue of the invention, in an amount effective to treat, reduce, and/or ameliorate the symptoms incurred by the GPCR-associated disease, disorder, syndrome, or condition. In some instances, an agonist or enhancer of a GPCR polypeptide or homologue of the invention is administered in an amount effective to treat and/or ameliorate the symptoms incurred by a GPCR-related disease, disorder, syndrome, or condition. In other instances, the administration of a novel GPCR polypeptide or homologue thereof pursuant to the present invention is envisioned for administration to treat a GPCR associated disease.

The polynucleotides or polypeptides, or agonists or antagonists of the present invention may be useful in treating, preventing, and/or diagnosing diseases, disorders, and/or conditions of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune diseases, disorders, and/or conditions may be genetic, somatic, such as cancer or some autoimmune diseases, disorders, and/or conditions, acquired (e.g., by chemotherapy or toxins), or infectious. Moreover, a polynucleotides or polypeptides, or agonists or antagonists of the present invention can be used as a marker or detector of a particular immune system disease or disorder.

A polynucleotides or polypeptides, or agonists or antagonists of the present invention may be useful in treating, preventing, and/or diagnosing diseases, disorders, and/or conditions of hematopoietic cells. A polynucleotides or polypeptides, or agonists or antagonists of the present invention could be used to increase differentiation and proliferation of hematopoietic cells, including the pluripotent stem cells, in an effort to treat or prevent those diseases, disorders, and/or conditions associated with a decrease in certain (or many) types hematopoietic cells. Examples of immunologic deficiency syndromes include, but are not limited to: blood protein diseases, disorders, and/or conditions (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a polynucleotides or polypeptides, or agonists or antagonists of the present invention could also be used to modulate hemostatic (the stopping of bleeding) or thrombolytic activity (clot formation). For example, by increasing hemostatic or thrombolytic activity, a polynucleotides or polypeptides, or agonists or antagonists of the present invention could be used to treat or prevent blood coagulation diseases, disorders, and/or conditions (e.g., afibrinogenemia, factor deficiencies, arterial thrombosis, venous thrombosis, etc.), blood platelet diseases, disorders, and/or conditions (e.g. thrombocytopenia), or wounds resulting from trauma, surgery, or other causes. Alternatively, a polynucleotides or polypeptides, or agonists or antagonists of the present invention that can decrease hemostatic or thrombolytic activity could be used to inhibit or dissolve clotting. Polynucleotides or polypeptides, or agonists or antagonists of the present invention are may also be useful for the detection, prognosis, treatment, and/or prevention of heart attacks (infarction), strokes, scarring, fibrinolysis, uncontrolled bleeding, uncontrolled coagulation, uncontrolled complement fixation, and/or inflammation.

A polynucleotides or polypeptides, or agonists or antagonists of the present invention may also be useful in treating, preventing, and/or diagnosing autoimmune diseases, disorders, and/or conditions. Many autoimmune diseases, disorders, and/or conditions result from inappropriate recognition of self as foreign material by immune cells. This inappropriate recognition results in an immune response leading to the destruction of the host tissue. Therefore, the administration of a polynucleotides or polypeptides, or agonists or antagonists of the present invention that inhibits an immune response, particularly the proliferation, differentiation; or chemotaxis of T-cells, may be an effective therapy in preventing autoimmune diseases, disorders, and/or conditions.

Examples of autoimmune diseases, disorders, and/or conditions that can be treated, prevented, and/or diagnosed or detected by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated, prevented, and/or diagnosed by polynucleotides or polypeptides, or agonists or antagonists of the present invention. Moreover, these molecules can be used to treat anaphylaxis, hypersensitivity to an antigenic molecule, or blood group incompatibility.

A polynucleotides or polypeptides, or agonists or antagonists of the present invention may also be used to treat, prevent, and/or diagnose organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a polynucleotides or polypeptides, or agonists or antagonists of the present invention that inhibits an immune response, particularly the proliferation, differentiation, or chemotaxis of T-cells, may be an effective therapy in preventing organ rejection or GVHD.

Similarly, a polynucleotides or polypeptides, or agonists or antagonists of the present invention may also be used to modulate inflammation. For example, the polypeptide or polynucleotide or agonists or antagonist may inhibit the proliferation and differentiation of cells involved in an inflammatory response. These molecules can be used to treat, prevent, and/or diagnose inflammatory conditions, both chronic and acute conditions, including chronic prostatitis, granulomatous prostatitis and malacoplakia, inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

A polynucleotides or polypeptides, or agonists or antagonists of the invention can be used to treat, prevent, and/or diagnose hyperproliferative diseases, disorders, and/or conditions, including neoplasms. A polynucleotides or polypeptides, or agonists or antagonists of the present invention may inhibit the proliferation of the disorder through direct or indirect interactions. Alternatively, a polynucleotides or polypeptides, or agonists or antagonists of the present invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative diseases, disorders, and/or conditions can be treated, prevented, and/or diagnosed. This immune response may be increased by either enhancing an existing immune response, or by initiating a new immune response. Alternatively, decreasing an immune response may also be a method of treating, preventing, and/or diagnosing hyperproliferative diseases, disorders, and/or conditions, such as a chemotherapeutic agent.

Examples of hyperproliferative diseases, disorders, and/or conditions that can be treated, prevented, and/or diagnosed by polynucleotides or polypeptides, or agonists or antagonists of the present invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative diseases, disorders, and/or conditions can also be treated, prevented, and/or diagnosed by a polynucleotides or polypeptides, or agonists or antagonists of the present invention. Examples of such hyperproliferative diseases, disorders, and/or conditions include, but are not limited to: hypergammaglobulinemia, lymphoproliferative diseases, disorders, and/or conditions, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

One preferred embodiment utilizes polynucleotides of the present invention to inhibit aberrant cellular division, by gene therapy using the present invention, and/or protein fusions or fragments thereof.

Thus, the present invention provides a method for treating or preventing cell proliferative diseases, disorders, and/or conditions by inserting into an abnormally proliferating cell a polynucleotide of the present invention, wherein said polynucleotide represses said expression.

Another embodiment of the present invention provides a method of treating or preventing cell-proliferative diseases, disorders, and/or conditions in individuals comprising administration of one or more active gene copies of the present invention to an abnormally proliferating cell or cells. In a preferred embodiment, polynucleotides of the present invention is a DNA construct comprising a recombinant expression vector effective in expressing a DNA sequence encoding said polynucleotides. In another preferred embodiment of the present invention, the DNA construct encoding the polynucleotides of the present invention is inserted into cells to be treated utilizing a retrovirus, or more Preferably an adenoviral vector (See G J. Nabel, et. al., PNAS 1999 96: 324-326, which is hereby incorporated by reference). In a most preferred embodiment, the viral vector is defective and will not transform non-proliferating cells, only proliferating cells. Moreover, in a preferred embodiment, the polynucleotides of the present invention inserted into proliferating cells either alone, or in combination with or fused to other polynucleotides, can then be modulated via an external stimulus (i.e. magnetic, specific small molecule, chemical, or drug administration, etc.), which acts upon the promoter upstream of said polynucleotides to induce expression of the encoded protein product. As such the beneficial therapeutic affect of the present invention may be expressly modulated (i.e. to increase, decrease, or inhibit expression of the present invention) based upon said external stimulus.

Polynucleotides of the present invention may be useful in repressing expression of oncogenic genes or antigens. By “repressing expression of the oncogenic genes” is intended the suppression of the transcription of the gene, the degradation of the gene transcript (pre-message RNA), the inhibition of splicing, the destruction of the messenger RNA, the prevention of the post-translational modifications of the protein, the destruction of the protein, or the inhibition of the normal function of the protein.

For local administration to abnormally proliferating cells, polynucleotides of the present invention may be administered by any method known to those of skill in the art including, but not limited to transfection, electroporation, microinjection of cells, or in vehicles such as liposomes, lipofectin, or as naked polynucleotides, or any other method described throughout the specification. The polynucleotide of the present invention may be delivered by known gene delivery systems such as, but not limited to, retroviral vectors (Gilboa, J. Virology 44:845 (1982); Hocke, Nature 320:275 (1986); Wilson, et al., Proc. Natl. Acad. Sci. U.S.A. 85:3014), vaccinia virus system (Chakrabarty et al., Mol. Cell Biol. 5:3403 (1985) or other efficient DNA delivery systems (Yates et al., Nature 313:812 (1985)) known to those skilled in the art. These references are exemplary only and are hereby incorporated by reference. In order to specifically deliver or transfect cells which are abnormally proliferating and spare non-dividing cells, it is preferable to utilize a retrovirus, or adenoviral (as described in the art and elsewhere herein) delivery system known to those of skill in the art. Since host DNA replication is required for retroviral DNA to integrate and the retrovirus will be unable to self replicate due to the lack of the retrovirus genes needed for its life cycle. Utilizing such a retroviral delivery system for polynucleotides of the present invention will target said gene and constructs to abnormally proliferating cells and will spare the non-dividing normal cells.

The polynucleotides of the present invention may be delivered directly to cell proliferative disorder/disease sites in internal organs, body cavities and the like by use of imaging devices used to guide an injecting needle directly to the disease site. The polynucleotides of the present invention may also be adrmnistered to disease sites at the time of surgical intervention.

By “cell proliferative disease” is meant any human or animal disease or disorder, affecting any one or any combination of organs, cavities, or body parts, which is characterized by single or multiple local abnormal proliferations of cells, groups of cells, or tissues, whether benign or malignant.

Any amount of the polynucleotides of the present invention may be administered as long as it has a biologically inhibiting effect on the proliferation of the treated cells. Moreover, it is possible to administer more than one of the polynucleotide of the present invention simultaneously to the same site. By “biologically inhibiting” is meant partial or total growth inhibition as well as decreases in the rate of proliferation or growth of the cells. The biologically inhibitory dose may be determined by assessing the effects of the polynucleotides of the present invention on target malignant or abnormally proliferating cell growth in tissue culture, tumor growth in animals and cell cultures, or any other method known to one of ordinary skill in the art.

The present invention is further directed to antibody-based therapies which involve administering of anti-polypeptides and anti-polynucleotide antibodies to a mammalian, preferably human, patient for treating, preventing, and/or diagnosing one or more of the described diseases, disorders, and/or conditions. Methods for producing anti-polypeptides and anti-polynucleotide antibodies polyclonal and monoclonal antibodies are described in detail elsewhere herein. Such antibodies may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

In particular, the antibodies, fragments and derivatives of the present invention are useful for treating, preventing, and/or diagnosing a subject having or developing cell proliferative and/or differentiation diseases, disorders, and/or conditions as described herein. Such treatment comprises administering a single or multiple doses of the antibody, or a fragment, derivative, or a conjugate thereof.

The antibodies of this invention may be advantageously utilized in combination with other monoclonal or chimeric antibodies, or with lymphokines or hematopoietic growth factors, for example, which serve to increase the number or activity of effector cells which interact with the antibodies.

It is preferred to use high affinity and/or potent in vivo inhibiting and/or neutralizing antibodies against polypeptides or polynucleotides of the present invention, fragments or regions thereof, for both immunoassays directed to and therapy of diseases, disorders, and/or conditions related to polynucleotides or polypeptides, including fragments thereof, of the present invention. Such antibodies, fragments, or regions, will preferably have an affinity for polynucleotides or polypeptides, including fragments thereof. Preferred binding affinities include those with a dissociation constant or Kd less than 5×10-6M, 10-6M, 5×10-7M, 10-7M, 5×10-8M, 10-8M, 5×10-9M, 10-9M, 5×10-10M, 10-10M, 5×10-11M, 10-11M, 5×10-12M, 10-12M, 5×10-13M, 10-13M, 5×10-14M, 10-14M, 5×10-15M, and 10-15M.

Moreover, polypeptides of the present invention may be useful in inhibiting the angiogenesis of proliferative cells or tissues, either alone, as a protein fusion, or in combination with other polypeptides directly or indirectly, as described elsewhere herein. In a most preferred embodiment, said anti-angiogenesis effect may be achieved indirectly, for example, through the inhibition of hematopoietic, tumor-specific cells, such as tumor-associated macrophages (See Joseph I B, et al. J Natl Cancer Inst, 90(21):1648-53 (1998), which is hereby incorporated by reference). Antibodies directed to polypeptides or polynucleotides of the present invention may also result in inhibition of angiogenesis directly, or indirectly (See Witte L, et al., Cancer Metastasis Rev. 17(2):155-61 (1998), which is hereby incorporated by reference)).

Polypeptides, including protein fusions, of the present invention, or fragments thereof may be useful in inhibiting proliferative cells or tissues through the induction of apoptosis. Said polypeptides may act either directly, or indirectly to induce apoptosis of proliferative cells and tissues, for example in the activation of a death-domain receptor, such as tumor necrosis factor (TNF) receptor-1, CD95 (Fas/APO-1), TNF-receptor-related apoptosis-mediated protein (TRAMP) and TNF-related apoptosis-inducing ligand (TRAIL) receptor-1 and -2 (See Schulze-Osthoff K, et al., Eur J Biochem 254(3):439-59 (1998), which is hereby incorporated by reference). Moreover, in another preferred embodiment of the present invention, said polypeptides may induce apoptosis through other mechanisms, such as in the activation of other proteins which will activate apoptosis, or through stimulating the expression of said proteins, either alone or in combination with small molecule drugs or adjuvants, such as apoptonin, galectins, thioredoxins, antiinflammatory proteins (See for example, Mutat. Res. 400(1-2):447-55 (1998), Med Hypotheses.50(5):423-33 (1998), Chem. Biol. Interact. April 24;111-112:23-34 (1998), J Mol Med.76(6):402-12 (1998), Int. J. Tissue React. 20(1):3-15 (1998), which are all hereby incorporated by reference).

Polypeptides, including protein fusions to, or fragments thereof, of the present invention are useful in inhibiting the metastasis of proliferative cells or tissues. Inhibition may occur as a direct result of administering polypeptides, or antibodies directed to said polypeptides as described elsewhere herein, or indirectly, such as activating the expression of proteins known to inhibit metastasis, for example alpha 4 integrins, (See, e.g., Curr Top Microbiol Immunol 1998;231:125-41, which is hereby incorporated by reference). Such therapeutic affects of the present invention may be achieved either alone, or in combination with small molecule drugs or adjuvants.

In another embodiment, the invention provides a method of delivering compositions containing the polypeptides of the invention (e.g., compositions containing polypeptides or polypeptide antibodies associated with heterologous polypeptides, heterologous nucleic acids, toxins, or prodrugs) to targeted cells expressing the polypeptide of the present invention. Polypeptides or polypeptide antibodies of the invention may be associated with heterologous polypeptides, heterologous nucleic acids, toxins, or prodrugs via hydrophobic, hydrophilic, ionic and/or covalent interactions.

Polypeptides, protein fusions to, or fragments thereof, of the present invention are useful in enhancing the immunogenicity and/or antigenicity of proliferating cells or tissues, either directly, such as would occur if the polypeptides of the present invention ‘vaccinated’ the immune response to respond to proliferative antigens and immunogens, or indirectly, such as in activating the expression of proteins known to enhance the immune response (e.g. chemokines), to said antigens and immunogens.

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B 12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

In yet another embodiment of the present invention, an expression vector containing the complement of the polynucleotide encoding a GPCR polypeptide is administered to an individual to treat or prevent any one of the types of diseases, disorders, or conditions previously described, in an antisense therapy method.

The GPCR proteins; modulators, including antagonists, antibodies, and agonists; complementary sequences; or vectors of the present invention can also be administered in combination with other appropriate therapeutic agents as necessary or desired. Selection of the appropriate agents for use in combination therapy may be made by the skilled practitioner in the art, according to conventional pharmaceutical and clinical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects or adverse events.

Antagonists or inhibitors of the GPCR polypeptide of this invention can be produced using methods which are generally known in the art. In particular, purified GPCR protein, or fragments thereof, can be used to produce antibodies, or to screen libraries of pharmaceutical agents, to identify those which specifically bind to the novel GPCR polypeptides as described herein.

Antibodies specific for GPCR polypeptide, or immunogenic peptide fragments thereof, can be generated using methods that have long been known and conventionally practiced in the art. Such antibodies may include, but are not limited to, polyclonal, monoclonal, neutralizing antibodies, (i.e., those which inhibit dimer formation), chimeric, single chain, Fab fragments, and fragments produced by an Fab expression library. Non-limiting examples of GPCR polypeptides or immunogenic fragments thereof that may be used to generate antibodies are provided in SEQ ID NO:2 and 4.

For the production of antibodies, various hosts, including goats, rabbits, sheep, rats, mice, humans, and others, can be immunized by injection with one or more of the GPCR polypeptides, or any immunogenic and/or epitope-containing fragment or oligopeptide thereof, which have immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guerin) and Corynebacterium parvumn.

Preferably, the GPCR polypeptides, peptides, fragments, or oligopeptides used to induce antibodies to the GPCR polypeptide immunogens have an amino acid sequence of at least five amino acids in length, and more preferably, at least 7-10, or more, amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of GPCR amino acids may be fused with another protein as carrier, such as KLH, such that antibodies are produced against the chimeric molecule.

Monoclonal antibodies to the GPCR polypeptides, or immunogenic fragments thereof, may be prepared using any technique which provides for the production of antibody molecules by continuous cell lines in culture. Such techniques are conventionally used in the art. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies to immunogenic proteins and peptides is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce GPCR polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments, which contain specific binding sites for a GPCR polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (e.g., W. D. Huse et al., 1989, Science, 254.1275-1281).

Various inimunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between a GPCR polypeptide and its specific antibody. A two-site, monoclonal-based immunoassay utilizing monoclonal antibodies reactive with two non-interfering GPCR polypeptide epitopes is suitable, but a competitive binding assay may also be employed (Maddox, supra).

To induce an immunological response in a mammal, a host animal is inoculated with a GPCR polypeptide, or a fragment thereof, of this invention in an amount adequate to produce an antibody and/or a T cell immune response to protect the animal from a disease or disorder associated with the expression or production of a GPCR polypeptide. Yet another aspect of the invention relates to a method of inducing immunological response in a mammal, if applicable or required. Such a method comprises delivering GPCR polypeptide via a vector directing expression of GPCR polynucleotide in vivo in order to induce such an immunological response to produce antibody to protect said animal from GPCR-related diseases.

A further aspect of the invention relates to an immunological vaccine or immunogen formulation or composition which, when introduced into a mammalian host, induces an immunological response in that mammal to a GPCR polypeptide wherein the composition comprises a GPCR polypeptide or GPCR gene. The vaccine or immunogen formulation may further comprise a suitable carrier. Since the GPCR polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents.

The formulations may be presented in unit-dose or multi-dose containers, for example, sealed ampoules and vials, and may be stored in a freeze-dried condition requiring only the addition of the sterile liquid carrier immediately prior to use. A vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In a specific embodiment, formulations of the present invention may further comprise antagonists of P-glycoprotein (also referred to as the multiresistance protein, or PGP), including antagonists of its encoding polynucleotides (e.g., antisense oligonucleotides, ribozymes, zinc-finger proteins, etc.). P-glycoprotein is well known for decreasing the efficacy of various drug administrations due to its ability to export intracellular levels of absorbed drug to the cell exterior. While this activity has been particularly pronounced in cancer cells in response to the administration of chemotherapy regimens, a variety of other cell types and the administration of other drug classes have been noted (e.g., T-cells and anti-HIV drugs). In fact, certain mutations in the PGP gene significantly reduces PGP function, making it less able to force drugs out of cells. People who have two versions of the mutated gene—one inherited from each parent—have more than four times less PGP than those with two normal versions of the gene. People may also have one normal gene and one mutated one. Certain ethnic populations have increased incidence of such PGP mutations. Among individuals from Ghana, Kenya, the Sudan, as well as African Americans, frequency of the normal gene ranged from 73% to 84%. In contrast, the frequency was 34% to 59% among British whites, Portuguese, Southwest Asian, Chinese, Filipino and Saudi populations. As a result, certain ethnic populations may require increased administration of PGP antagonist in the formulation of the present invention to arrive at the an efficacious dose of the therapeutic (e.g., those from African descent). Conversely, certain ethnic populations, particularly those having increased frequency of the mutated PGP (e.g., of Caucasian descent, or non-African descent) may require less pharmaceutical compositions in the formulation due to an effective increase in efficacy of such compositions as a result of the increased effective absorption (e.g., less PGP activity) of said composition.

Moreover, in another specific embodiment, formulations of the present invention may further comprise antagonists of OATP2 (also referred to as the multiresistance protein, or MRP2), including antagonists of its encoding polynucleotides (e.g., antisense oligonucleotides, ribozymes, zinc-finger proteins, etc.). The invention also further comprises any additional antagonists known to inhibit proteins thought to be attributable to a multidrug resistant phenotype in proliferating cells.

Preferred antagonists that formulations of the present may comprise include the potent P-glycoprotein inhibitor elacridar, and/or LY-335979. Other P-glycoprotein inhibitors known in the art are also encompassed by the present invention.

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219(1981); and Nygren, J. Histochem. And Cytochem., 30:407 (1982).

Antibodies directed against the polypeptides of the present invention are useful for the affinity purification of such polypeptides from recombinant cell culture or natural sources. In this process, the antibodies against a particular polypeptide are immobilized on a suitable support, such as a Sephadex resin or filter paper, using methods well known in the art. The immobilized antibody then is contacted with a sample containing the polypeptides to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide from the antibody.

### Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

### Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

### Therapeutic Uses Of Antibodies

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6 M, 5×10-7 M, 10-7 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, and 10-15 M.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the gene encoding an antibody directed against a polypeptide of the present invention, said polypeptide having the potential to elicit an allergic and/or immune response in an organism, and transforming the organism with said antibody gene such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or gene therapy applications of the present invention are provided elsewhere herein.

Alternatively, antibodies of the present invention could be produced in a plant (e.g., cloning the gene of the antibody directed against a polypeptide of the present invention, and transforming a plant with a suitable vector comprising said gene for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

In another embodiment, antibodies of the present invention, preferably polyclonal antibodies, more preferably monoclonal antibodies, and most preferably single-chain antibodies, can be used as a means of inhibiting gene expression of a particular gene, or genes, in a human, mammal, and/or other organism. See, for example, International Publication Number WO 00/05391, published Feb. 3, 2000, to Dow Agrosciences LLC. The application of such methods for the antibodies of the present invention are known in the art, and are more particularly described elsewhere herein.

In yet another embodiment, antibodies of the present invention may be useful for multimerizing the polypeptides of the present invention. For example, certain proteins may confer enhanced biological activity when present in a multimeric state (i.e., such enhanced activity may be due to the increased effective concentration of such proteins whereby more protein is available in a localized location).

### Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. Demonstration of Therapeutic or

Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem . . . 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diagnosis and Imaging With Antibodies

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S.W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

### Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

In an aspect of the present invention, the polynucleotide encoding a GPCR polypeptide, or any fragment or complement thereof, as described herein may be used for therapeutic purposes. For instance, antisense to a GPCR polynucleotide encoding a GPCR polypeptide, may be used in situations in which it would be desirable to block the transcription of GPCR mRNA. In particular, cells may be transformed, transfected, or injected with sequences complementary to polynucleotides encoding GPCR polypeptide. Thus, complementary molecules may be used to modulate GPCR polynucleotide and polypeptide activity, or to achieve regulation of gene function. Such technology is well known in the art, and sense or antisense oligomers or oligonucleotides, or larger fragments, can be designed from various locations along the coding or control regions of the GPCR polynucleotide sequences encoding the novel GPCR polypeptides.

Polypeptides used in treatment can also be generated endogenously in the subject, in treatment modalities often referred to as “gene therapy”. Thus for example, cells from a subject may be engineered with a polynucleotide, such as DNA or RNA, to encode a polypeptide ex vivo, for example, by the use of a retroviral plasmid vector. The cells can then be introduced into the subject's body in which the desired polypeptide is expressed.

A gene encoding a GPCR polypeptide can be turned off by transforming a cell or tissue with an expression vector that expresses high levels of a GPCR polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of a GPCR polynucleotide sequence encoding a GPCR polypeptide, (e.g., a signal sequence, promoters, enhancers, and introns). Oligonucleotides may be derived from the transcription initiation site, for example, between positions −10 and +10 from the start site.

Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Many methods for introducing vectors into cells or tissues are available and are equally suitable for use in vivo, in vitro, and ex vivo. For ex vivo therapy, vectors may be introduced into stem cells or bone marrow cells obtained from the patient and clonally propagated for autologous transplant back into that same patient. Delivery by transfection, direct injection (e.g., microparticle bombardment) and by liposome injections may be achieved using methods which are well known in the art.

Any of the therapeutic methods described above can be applied to any individual in need of such therapy, including, for example, mammals such as dogs, cats, cows, horses, rabbits, monkeys, and most preferably, humans.

### Administration

A further embodiment of the present invention embraces the administration of a pharmaceutical composition, in conjunction with a pharmaceutically acceptable carrier, diluent, or excipient, to achieve any of the above-described therapeutic uses and effects. Such pharmaceutical compositions can comprise GPCR nucleic acid, polypeptide, or peptides, antibodies to GPCR polypeptide, mimetics, GPCR modulators, such as agonists, antagonists, or inhibitors of a GPCR polypeptide or polynucleotide, preferably the HGPRBM7e1 and/or HGPRBMY31 variant having polypeptide SEQ ID NO:2 and 4, respectively, and polynucleotide SEQ ID NO:1 and 3, respectively. The compositions can be administered alone, or in combination with at least one other agent or reagent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (e.g., GPCR nucleic acid or polypeptide, or functional fragments thereof, or a GPCR agonist or antagonist), the pharmaceutical compositions may contain pharmaceutically acceptable/physiologically suitable carriers or excipients comprising auxiliaries which facilitate processing of the active compounds into preparations that can be used pharmaceutically. Further details on techniques for formulation and administration are provided in the latest edition of Remington 's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

In addition, pharmaceutical preparations for oral use can be obtained by the combination of active compounds with a solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, further include push-fit capsules made of gelatin, as well as soft, scaled capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents (enhancers) that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

A pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of a GPCR product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, for example, using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, GPCR polynucleotide, GPCR polypeptide, or fragments thereof, antibodies to GPCR polypeptide, agonists, antagonists or inhibitors of GPCR polypeptide, which ameliorates, reduces, diminishes, or eliminates the symptoms or condition. Therapeutic efficacy and toxicity can be determined by standard pharmaceutical procedures in cell cultures or in experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, the sensitivity of the patient, and the route of administration.

The practitioner, who will consider the factors related to an individual requiring treatment, will determine the exact dosage. Dosage and administration are adjusted to provide sufficient levels of the active component, or to maintain the desired effect. Factors which may be taken into account include the severity of the individual's disease state; the general health of the patient; the age, weight, and gender of the patient; diet; time and frequency of administration; drug combination(s); reaction sensitivities; and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

As a guide, normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors or activators. Similarly, the delivery of polynucleotides or polypeptides will be specific to particular cells, conditions, locations, and the like.

### Microarrays and Screening Assays

In another embodiment of the present invention, oligonucleotides, or longer fragments derived from the GPCR polynucleotide sequences described herein can be used as targets in a microarray. The microarray can be used to monitor the expression levels of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 to Chee et al.; D. J. Lockhart et al., 1996, Nature Biotechnology, 14:1675-1680; and M. Schena et al., 1996, Proc. Natl. Acad. Sci. USA, 93:10614-10619. Microarrays are further described in U.S. Pat. No. 6,015,702 to P. Lal et al.

In another embodiment of this invention, a nucleic acid sequence which encodes a novel GPCR polypeptide, may also be used to generate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

In another embodiment of the present invention, a GPCR polypeptide of this invention, its catalytic or immunogenic fragments, or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between the GPCR polypeptide, or a portion thereof, and the agent being tested, may be measured utilizing techniques commonly practiced in the art.

The human HGPRBMY31 polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a HGPRBMY31 polypeptide, or a bindable peptide fragment, of this invention, comprising providing a plurality of compounds, combining the HGPRBMY31 polypeptide, or a bindable peptide fragment, with each of a plurality of compounds for a time sufficient to allow binding under suitable conditions and detecting binding of the HGPRBMY31 polypeptide or peptide to each of the plurality of test compounds, thereby identifying the compounds that specifically bind to the HGPRBMY31 polypeptide or peptide.

Methods of identifying compounds that modulate the activity of the novel human HGPRBMY31 polypeptides and/or peptides are provided by the present invention and comprise combining a potential or candidate compound or drug modulator of G-protein coupled receptor biological activity with an HGPRBMY31 polypeptide or peptide, for example, the HGPRBMY31 amino acid sequence as set forth in SEQ ID NO:2, and measuring an effect of the candidate compound or drug modulator on the biological activity of the HGPRBMY31 polypeptide or peptide. Such measurable effects include, for example, physical binding interaction; the ability to cleave a suitable G-protein coupled receptor substrate; effects on native and cloned HGPRBMY31-expressing cell line; and effects of modulators or other G-protein coupled receptor-mediated physiological measures.

Another method of identifying compounds that modulate the biological activity of the novel HGPRBMY31 polypeptides of the present invention comprises combining a potential or candidate compound or drug modulator of a G-protein coupled receptor biological activity with a host cell that expresses the HGPRBMY31 polypeptide and measuring an effect of the candidate compound or drug modulator on the biological activity of the HGPRBMY31 polypeptide. The host cell can also be capable of being induced to express the HGPRBMY31 polypeptide, e.g., via inducible expression. Physiological effects of a given modulator candidate on the HGPRBMY31 polypeptide can also be measured. Thus, cellular assays for particular G-protein coupled receptor modulators may be either direct measurement or quantification of the physical biological activity of the HGPRBMY31 polypeptide, or they may be measurement or quantification of a physiological effect. Such methods preferably employ a HGPRBMY31 polypeptide as described herein, or an overexpressed recombinant HGPRBMY31 polypeptide in suitable host cells containing an expression vector as described herein, wherein the HGPRBMY31 polypeptide is expressed, overexpressed, or undergoes upregulated expression.

Another aspect of the present invention embraces a method of screening for a compound that is capable of modulating the biological activity of a HGPRBMY31 polypeptide, comprising providing a host cell containing an expression vector harboring a nucleic acid sequence encoding a HGPRBMY31 polypeptide, or a functional peptide or portion thereof (e.g., SEQ ID NOS:2); determining the biological activity of the expressed HGPRBMY31 polypeptide in the absence of a modulator compound; contacting the cell with the modulator compound and determining the biological activity of the expressed HGPRBMY31 polypeptide in the presence of the modulator compound. In such a method, a difference between the activity of the HGPRBMY31 polypeptide in the presence of the modulator compound and in the absence of the modulator compound indicates a modulating effect of the compound.

Essentially any chemical compound can be employed as a potential modulator or ligand in the assays according to the present invention. Compounds tested as G-protein coupled receptor modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel HGPRBMY31 polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptides (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a HGPRBMY31 polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a HGPRBMY31 polypeptide or peptide to measure a biological binding or ligand binding activity, the source may be a whole cell lysate that can be prepared by successive freeze-thaw cycles (e.g., one to three) in the presence of standard protease inhibitors. The HGPRBMY31 polypeptide may be partially or completely purified by standard protein purification methods, e.g., affinity chromatography using specific antibody described infra, or by ligands specific for an epitope tag engineered into the recombinant HGPRBMY31 polypeptide molecule, also as described herein. Binding activity can then be measured as described.

Compounds which are identified according to the methods provided herein, and which modulate or regulate the biological activity or physiology of the HGPRBMY31 polypeptides according to the present invention are a preferred embodiment of this invention. It is contemplated that such modulatory compounds may be employed in treatment and therapeutic methods for treating a condition that is mediated by the novel HGPRBMY31 polypeptides by administering to an individual in need of such treatment a therapeutically effective amount of the compound identified by the methods described herein.

In addition, the present invention provides methods for treating an individual inneed of such treatment for a disease, disorder, or condition that is mediated by the HGPRBMY31 polypeptides of the invention, comprising administering to the individual a therapeutically effective amount of the HGPRBMY31-modulating compound identified by a method provided herein.

Another technique for drug screening, which may be employed, provides for high throughput screening of compounds having suitable binding affinity to the protein of interest as described in WO 84/03564 to Venton, et al. In this method, as applied to the GPCR protein, large numbers of different small test compounds are synthesized on a solid substrate, such as plastic pins or some other surface. The test compounds are reacted with the GPCR polypeptide, or fragments thereof, and washed. Bound GPCR polypeptide is then detected by methods well known in the art. Purified GPCR polypeptide can also be coated directly onto plates for use in the aforementioned drug screening techniques. Alternatively, non-neutralizing antibodies can be used to capture the peptide and immobilize it on a solid support.

In a further embodiment, competitive drug screening assays can be used in which neutralizing antibodies, capable of binding a GPCR polypeptide according to this invention, specifically compete with a test compound for binding to the GPCR polypeptide. In this manner, the antibodies can be used to detect the presence of any peptide that shares one or more antigenic determinants with the GPCR polypeptide.

A polypeptide of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins. minerals, cofactors or other nutritional factors or component(s); effecting behavioral characteristics, including, without limitation, appetite, libido, stress, cognition (including cognitive disorders), depression (including depressive disorders) and violent behaviors, analgesic effects or other pain reducing effects; promoting differentiation and growth of embryonic stem cells in lineages other than hernatopoletic lineages; hormonal or endocrine activity; in the case of enzymes, correcting deficiencies of the enzyme and treating deficiencv-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Polypeptide or polynucleotides and/or agonist or antagonists of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. Such a use may be administered either prior to an extraterrestrial event, during an extraterrestrial event, or both. Moreover, such a use may result in a number of beneficial changes in the recipient, such as, for example, any one of the following, non-limiting, effects: an increased level of hematopoietic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoietic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid the recipient by decreasing their basal daily energy requirements; (ii) effectively lower the level of oxidative and/or metabolic stress in recipient (i.e., to enable recipient to cope with increased extraterrestial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: A Laboratory Manual, 2^{nd} Edition, Cold Spring Harbor Laboratory Press, USA, (1989).

### Example 1

### Bioinformatics Analysis

G-protein coupled receptor sequences were used as probes to search the human genomic sequence database. The search program used was gapped BLAST (Altschul et al. Nuc. Acids Res. 25:3389-3402, 1997). The top genomic exon hits from the BLAST results were searched back against the non-redundant protein and patent sequence databases. From this analysis, exons encoding potential novel GPCRs were identified based on sequence homology. Also, the genomic region surrounding the matching exons was analyzed. Based on this analysis, potential full-length sequence of a novel human GPCR, HGPRBMY31, also called GPCR61 was identified directly from the genomic sequence (Genbank Accession no. AP000808). The full-length clone of this GPCR was experimentally obtained using the sequence from genomic data. The complete protein sequence of HGPRBMY31 was analyzed for potential transmembrane domains. TMPRED program (Hofmann, K. and W. Stoffel Biol. Chem. Hoppe-Seyler 347:166, 1993) was used for transmembrane prediction. Also, a variant form of HGPRBMY31, called HGPRBMY31 variant, has been predicted directly from the genomic data. The human BAC: AP000808 was used to predict the variant sequence.

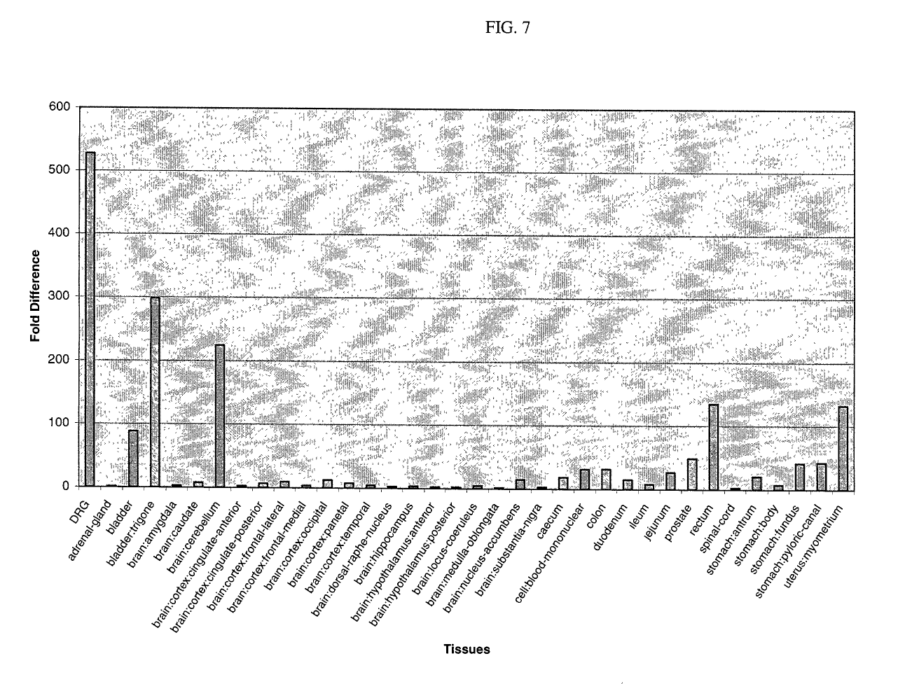

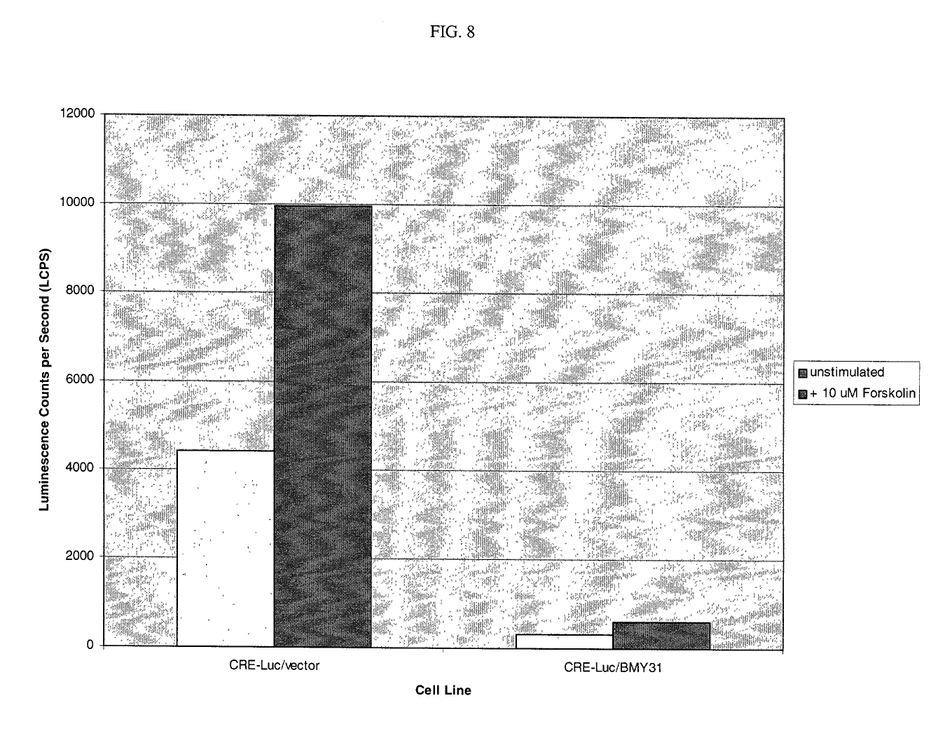

Domain predictions as shown in FIGS. 7 and 8, are valuable for suggesting possible functional domains in the predicted protein. These predictions are based on comparisons of the given protein sequence (the query, or Q) against a collection of statistical models known as Hidden Markov Models (HMMs) (the targets, or T). HMMs represent consensus patterns for known functional domains and this method of comparison allows for the prediction of functional domains in novel protein sequences. In particular, the HGPRBMY31 and HGPRBMY31 variant were searched against profile hidden Markov models of GPCRs. Profile hidden Markov models (profile HMMs) are built from the Pfam alignments. The Pfam is a database of multiple alignments of protein domains or conserved protein regions. The alignments represent some evolutionary conserved structure, which has implications for the protein's function that can be very useful for automatically recognizing that a new protein belongs to an existing protein family, even if the homology is weak (A. Bateman, E. Birney, R. Durbin, S. R. Eddy, K. L. Howe, and E. L. L. Sonnhammer. The Pfam Protein Families Database. Nucleic Acids Research, 28:263-266, 2000). HGPRBMY31 and HGPRBMY31 variant matched significantly to the “CLASS A” Rhodopsin GPCRs Pfam (FIGS. 7 and 8). Based on sequence, structure and significant match to the GPCR Pfam domain, the orphan protein HGPRBMY31 and HGPRBMY31 variant are predicted to be novel human GPCRs.

In FIGS. 7 and 8, the query (or “Q”) sequence is that of HGPRBMY31 and HGPRBMY31 variant, respectively, while the target (“T”) sequence is that of the sequence having the highest percent identity, i.e., seven transmembrane receptor of the rhodopsin family, for this GPCR sequence.

### Example 2

### Cloning of the Novel Human GPCR, HGPRBMY31

Using the predicted gene sequence from BAC: AP000808, an antisense 80 base pair oligonucleotide with biotin on the 5′ end complementary to the putative coding region of GPCR was designed as follows:

5-b-ATCTTCCTCTCGTAGGGATGAACCAGACTTTGAATAGCAGTGGGACCG TGGAGTCAGCCCTAAACTATTCCAGAGGGAG-3′ (SEQ ID NO:16).

This biotinylated oligo can be incubated with a mixture of single-stranded covalently closed circular cDNA libraries, which contain DNA corresponding to the sense strand. Hybrids between the biotinylated oligo and the circular cDNA are captured on streptavidin magnetic beads. Upon thermal release of the cDNA from the biotinylated oligo, the single stranded cDNA is converted into double strands using a primer homologous to a sequence on the cDNA cloning vector. The double stranded cDNA is introduced into E. coli by electroporation and the resulting colonies are screened by PCR, using a primer pair designed from the EST sequence to identify the proper cDNA. Oligos used to identify the cDNA of HGPRBMY31 by PCR were as follows:

| | |
|---|---|
| HGRBMY31s | |
| 5′-TCTCGTAGGGATGAACCAGAC-3′ | (SEQ ID NO:17) |
| HGRBMY31a | |
| 5′-CACGGTCCCACTGCTATTC-3′ | (SEQ ID NO:18) |

### Example 3

### Multiplex Cloning

Construction Of Size Fractionated cDNA Libraries Brain and testis polyA+ RNA was purchased from Clontech, treated with DNase I to remove traces of genomic DNA contamination, and converted into double stranded cDNA using the SuperScript™ Plasmid System for cDNA Synthesis and Plasmid Cloning (Life Technologies). No radioisotope was incorporated in either of the cDNA synthesis steps. The cDNA was then size fractionated on a TransGenomics HPLC system equipped with a size exclusion column (TosoHass) with dimensions of 7.8 mm×30 cm and a particle size of 10 μm. Tris buffered saline (TBS) was used as the mobile phase, and the column was run at a flow rate of 0.5 mL/min. The system was calibrated by running a 1 kb ladder through the column and analyzing the fractions by agarose gel electrophoresis. Using these data, it can be determined which fractions are to be pooled to obtain the largest cDNA library. Generally, fractions that eluted in the range of 12 to 15 minutes were used.

The cDNA was precipitated, concentrated and then ligated into the SalI/NotI sites in pSPORT. After electroporation into E. coli DH12S, colonies were subjected to a miniprep procedure and the resulting cDNA was digested using SalI/NotI restriction enzymes. Generally, the average insert size of libraries made in this fashion was greater the 3.5 Kb; the overall complexity of the library is optimally greater than 10⁷ independent clones. The library was amplified in semi-solid agar for 2 days at 30° C. An aliquot (200 microliters) of the amplified library was inoculated into a 200 mL culture for single-stranded DNA isolation by super-infection with an f1 helper phage. After overnight growth, the released phage particles were precipitated with PEG and the DNA isolated with proteinase K, SDS, and phenol extractions. The single stranded circular DNA was concentrated by ethanol precipitation, resuspended at a concentration of one microgram per microliter and used for the cDNA capture experiments.

### Conversion of Double-Stranded cDNA Libraries into Single-Stranded Circular Form

To prepare cultures, 200 mL LB with 400 μL carbenicillin (100 mg/mL stock solution) was inoculated with from 200 μL to 1 mL of thawed cDNA library and incubated at 37° C. while shaking at 250 rpm for approximately 45 minutes, or until an OD600 of 0.025-0.040 was attained. M13K07 helper phage (1 mL) was added to the culture and grown for 2 hours, after which kanamycin (500 μl; 30 mg/mL) was added and the culture was grown for an additional 15-18 hours.

The culture was then poured into 6 screw-cap tubes (50 mL autoclaved tubes) and cells subjected to centrifugation at 10K in an HB-6 rotor for 15 minutes at 4° C. to pellet the cells. The supernatant was filtered through a 0.2 μm filter and 12,000 units of Gibco DNase I was added. The mixture was incubated for 90 minutes at room temperature.

For PEG precipitation, 50 mL of ice-cold 40% PEG 8000, 2.5 M NaCl, and 10 mM MgSO₄ were added to the supernatant, mixed, and aliquotted into 6 centrifuge tubes (covered with parafilm). The tubes and contents were incubated for 1 hour on wet ice or at 4° C. overnight. The tubes were then centrifuged at 10K in a HB-6 rotor for 20 minutes at 4° C. to pellet the helper phage.

Following centrifugation, the supernatant was discarded and the sides of the tubes were dried. Each pellet was resuspended in 1 mL TE, pH 8. The resuspended pellets were pooled into a 14 mL tube (Sarstadt) containing 6 mL total. SDS was added to 0.1% (60 μl of stock 10% SDS). Freshly made proteinase K (20 mg/mL) was added (60 μl) and the suspension was incubated for 1 hour at 42° C.

For phenol/chloroform extractions, 1 mL of NaCl (5M) was added to the suspension in the tube. An equal volume of phenol/chloroform (6 mL) was added and the contents were vortexed or shaken. The suspension was then centrifuged at 5K in an HB-6 rotor for 5 minutes at 4° C. The aqueous (top) phase was transferred to a new tube (Sarstadt) and extractions were repeated until no interface was visible.

Ethanol precipitation was then performed on the aqueous phase whose volume was divided into 2 tubes containing 3 mL each. To each tube, 2 volumes of 100% ethanol was added and precipitation was carried out overnight at −20° C. The precipitated DNA was pelleted at 10K in an HB-6 rotor for 20 minutes at 4° C. The ethanol was discarded. Each pellet was resuspended in 700 μl of 70% ethanol. The contents of each tube were combined into one microcentrifuge tube and centrifuged in a microcentrifuge (Eppendorf) at 14K for 10 minutes at 4° C. After discarding the ethanol, the DNA pellet was dried in a speed vacuum. In order to remove oligosaccharides, the pellet was resuspended in 50 μl TE buffer, pH 8. The resuspension was incubated on dry ice for 10 minutes and centrifuged at 14K in an Eppendorf microfuge for 15 minutes at 4° C. The supernatant was then transferred to a new tube and the final volume was recorded.

To check purity, DNA was diluted 1:100 and added to a micro quartz cuvette, where DNA was analyzed by spectrometry at an OD260/OD280. The preferred purity ratio was between 1.7 and 2.0. The DNA was diluted to 1 μg/1L in TE, pH 8 and stored at 4° C. The concentration of DNA was calculated using the formula: (32 μg/mL*OD)(mL/1000 μL)(100)(OD260). The quality of single-stranded DNA was determined by first mixing 1L of 5 ng/μl ssDNA; 1 μl deionized water; 1.5 μL 10 μM T7 sport primer (fresh dilution of stock); 1.5 l, 10× Precision-Taq buffer per reaction. In the repair mix, a cocktail of 4 μl of 5 mM dNTPs (1.25 mM each); 1.5 μL 10× Precision-Taq buffer; 9.25 μL deionized water; and 0.25 μL Precision-Taq polymerase was mixed per reaction and preheated at 70° C. until the middle of the thermal cycle.

The DNA mixes were aliquotted into PCR tubes and the thermal cycle was started. The PCR thermal cycle consisted of 1 cycle at 95° C. for 20 sec.; 59° C. for 1 min. (15 μL repair mix added); and 73° C. for 23 minutes. For ethanol precipitation, 15 μg glycogen, 16 μl ammonium acetate (7.5M), and 125 μL 100% ethanol were added and the contents were centrifuged at 14K in an Eppendorf microfuge for 30 minutes at 4° C. The resulting pellet was washed one time with 125 μL 70% ethanol and then the ethanol was discarded. The pellet was dried in a speed vacuum and resuspended in 10 μL TE buffer, pH 8.

Single-stranded DNA was electroporated into E. coli DH10B or DH12S cells by pre-chilling the cuvettes and sliding holder and thawing the cells on ice-water. DNA was aliquotted into micro centrifuge tubes (Eppendorf) as follows: 2 μL repaired library, (=1×1⁻³ Hg); 1 μL unrepaired library (1 ng/μL), (=1×10⁻³ μg); and 1 μL pUC19 positive control DNA (0.01 μg/μL), (=1×10⁻⁵ μg). The mixtures were stored on ice until use.

One at a time, 40 μL of cells were added to a DNA aliquot. The cell/DNA mixture was not pipetted up and down more than one time. The mixture was then transferred via pipette into a cuvette between the metal plates and electroporation was performed at 1.8 kV. Immediately afterward, 1 mL SOC medium (i.e., SOB (bacto-tryptone; bacto-yeast extract; NaCl)+glucose (20 mM)+Mg²⁺) (See, J. Sambrook et al., Molecular Cloning: A Laboratory Manual, 2nd Ed., A.2, 1989) was added to the cuvette and the contents were transferred 15 mL media as commonly known in the art. The cells were allowed to recover for 1 hour at 37° C. with shaking (225 rpm).

Serial dilutions of the culture were made in 1:10 increments (20 μL into 180 μL LB) for plating the electroporated cells. For the repaired library, dilutions of 1:100, 1:1000, 1:10,000 were made. For the unrepaired library, dilutions of 1:10 and 1:100 were made. Positive control dilutions of 1:10 and 1:100 were made. Each dilution (100 μL) was plated onto small plates containing LB+carbenicillin and incubated at 37° C. overnight. The titer and background were calculated by methods well known in the art. Specifically, the colonies on each plate were counted using the lowest dilution countable. The titer was calculated using the formula: (# of colonies)(dilution factor)(200 μL/¹⁰⁰ μL)(1000 μL/20 μL)=CFUs, where CFUs/μg DNA used=CFU/μg. The % background=((unrepaired CFU/μg)/(repaired CFU/μg))×100%.

### Solution Hybridization And DNA Capture

One microliter (150 ng) of the anti-sense biotinylated oligonucleotide (SEQ ID NO:16) was added to six microliters (6 μg) of a mixture of single-stranded, covalently-closed, circular brain and testis cDNA libraries, and seven microliters of 100% formamide in a 0.5 mL PCR tube. The mixture was heated in a thermal cycler to 95° C. for 2 minutes. Fourteen microliters of 2× hybridization buffer (50% formamide, 1.5 M NaCl, 0.04 M NaPO₄, pH 7.2, 5 mM EDTA, 0.2% SDS) were added to the heated probe/cDNA library mixture and incubated at 42° C. for 26 hours. Hybrids between the biotinylated oligo and the circular cDNA were isolated by diluting the hybridization mixture to 220 microliters in a solution containing 1 M NaCl, 10 mM Tris-HCl pH 7.5, 1 mM EDTA, pH 8.0 and adding 125 microliters of streptavidin magnetic beads. This solution was incubated at 42° C. for 60 minutes, and mixed every 5 minutes to resuspend the beads. The beads were separated from the solution with a magnet and washed three times in 200 microliters of 0.1× SSPE, 0.1% SDS at 45° C.

The single stranded cDNAs were released from the biotinylated oligo/streptavidin magnetic bead complex by adding 50 microliters of 0.1 N NaOH and incubating at room temperature for 10 minutes. Six microliters of 3 M sodium acetate was added along with 15 micrograms of glycogen and the solution was ethanol precipitated with 120 microliters of 100% ethanol. The precipitated DNA was re-suspended in 12 microliters of TE (10 mM Tris-HCl, pH 8.0, 1 nM EDTA, pH 8.0). The single stranded cDNA was converted into double strands in a thermal cycler by mixing 5 microliters of the captured DNA with 1.5 microliters of 10 μM of standard SP6 primer for libraries in pSPORT 1, and 1.5 μL of 10× PCR buffer. The mixture was heated to 95° C. for 20 seconds, and then ramped down to 59° C. At this time, 15 μL of a repair mix (4 μL of 5 mM dNTPs (1.25 mM each); 1.5 μL of 10× PCR buffer; 9.25 μL of water; and 0.25 μL of Taq polymerase) that was preheated to 70° C., was added to the DNA. The solution was ramped back to 73° C. and incubated for 23 minutes.

The repaired DNA was ethanol precipitated and resuspended in 10 μL of TE. Two microliters were electroporated per tube containing 40 μL of E. coli DH12S cells. Three hundred and thirty three μL were plated onto one 150 mm plate of LB agar plus 100 μg/mL of ampicillin. After overnight incubation at 37° C., the resulting colonies from all plates were harvested by scraping into 10 mL of LB+50 μg/mL of ampicillin and 2 mL of sterile glycerol. The resulting colonies were screened by PCR using a primer pair designed from the genomic exonic sequence to identify the proper cDNAs. The oligos used to identify the cDNA by PCR are, for example, the primers having SEQ ID NO:17-18.

### Example 4

### Expression Profiling of Novel Human GPCR Polypeptides

The same PCR primer pairs used to identify GPCR cDNA clones were used to measure the steady state levels of mRNA by quantitative PCR. Briefly, first strand cDNA is made from commercially available mRNA (Clontech) and subjected to real time quantitative PCR using a PE 5700 instrument (Applied Biosystems, Foster City, Calif.) which detects the amount of DNA amplified during each cycle by the fluorescent output of SYBR green, a DNA binding dye specific for double strands. The specificity of the primer pair for its target is verified by performing a thermal denaturation profile at the end of the run which provided an indication of the number of different DNA sequences present by determining melting Tm. The contribution of contaminating genomic DNA to the assessment of tissue abundance is controlled for by performing the PCR with first strand made with and without reverse transcriptase. In all cases, the contribution of material amplified in the no reverse transcriptase controls is expected to be negligible.

Small variations in the amount of cDNA used in each tube are determined by performing a parallel experiment using a primer pair for the cyclophilin gene, which is expressed in equal amounts in all tissues. These data were used to normalize the data obtained with the primer pairs. The PCR data were converted into a relative assessment of the differences in transcript abundance among the tissues tested.

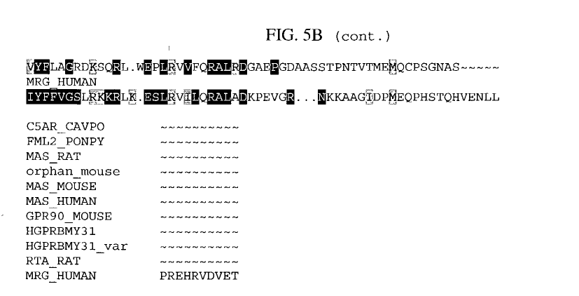

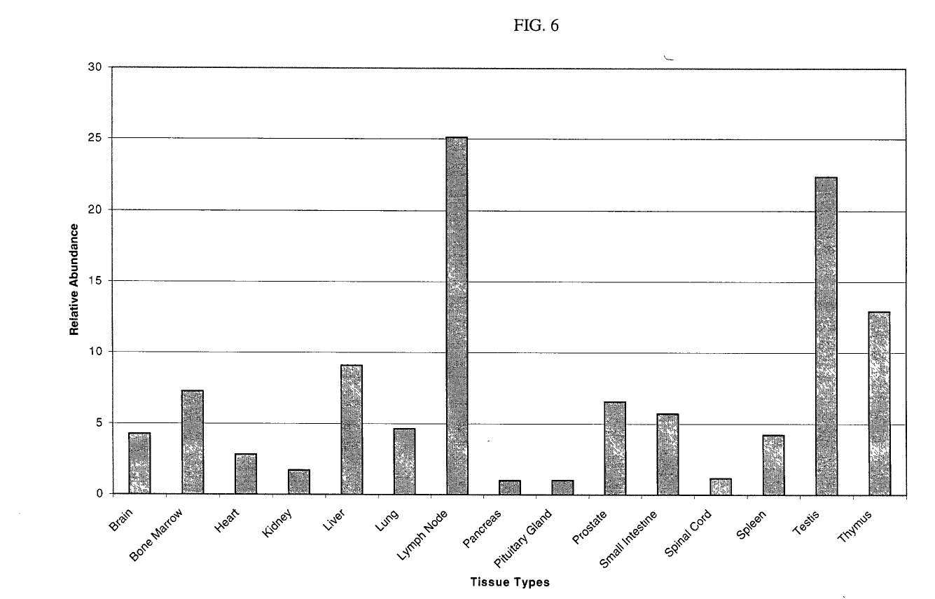

As indicated in FIG. 6, transcripts corresponding to HGPRBMY31 as described herein were found to be expressed in several tissues of pharmacological interest, but not limited to heart, brain, pituitary, thymus, testis, lymph node, small intestine, prostate, and bone marrow.

### Example 5

### Method of Assessing the Expression Profile of the Novel HGPRBMY31 Polypeptides of the Present Invention Using Expanded mRNA Tissue and Cell Sources

Total RNA from tissues was isolated using the TriZol protocol (Invitrogen) and quantified by determining its absorbance at 260 nM. An assessment of the 18s and 28s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For HGPRBMY31, the primer probe sequences were as follows

| | |
|---|---|
| Forward Primer | |
| 5′-CCATGCTTCACAACCCTTCTC-3′ | (SEQ ID NO:5) |
| Reverse Primer | |
| 5′-ACCAACCGTCGGCTTTAGACT-3′ | (SEQ ID NO:6) |
| TaqMan Probe | |
| 5′-CCACCTCGTGGCTGCCCAGGT-3′ | (SEQ ID NO:32) |

I. DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+ RNA. If not the RNA was not used in actual experiments.

II. Reverse Transcription Reaction and Sequence DetectioN

100 ng of Dnase-treated total RNA was annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 2.0 μM of the TaqMan probe, 500 μM of each dNTP, buffer and 5U AmpliTaq Gold™. The PCR reaction was then held at 94° C. for 12 min, followed by 40 cycles of 94° C. for 15 sec and 60° C. for 30 sec.

III. Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(Δct)}

SYBR green quantitative PCR analysis of HGPRBMY31 demonstrated that transcripts for this gene could be found in a variety of tissues albeit at low abundance, as shown in FIG. 6 and described in Example 10. Analysis of HGPRBMY31 by TaqMan™ quantitative PCR on an extended panel of tissue RNAs confirms that this gene is indeed expressed at low levels, but with a degree of specificity not previously appreciated. The tissue with the highest level of expression was the dorsal root ganglion, where transcripts for HGPRBMY31 can be found at levels 500 times greater that most other tissues. Expression in the neighboring spinal cord was essentially absent. Within the brain, expression of HGPRBMY31 was essentially restricted to the cerebellum, where transcripts were found in approximately 200 fold greater abundance than the 17 other sub regions analyzed. Tissues that also showed higher than average expression levels were the bladder trigone and the bladder itself. Low level HGPRBMY31 expression was also observed throughout the gastrointestinal tract with the greatest transcript numbers being observed in the rectum.

### Example 6

### Functional Characterization of the Novel Human GPCR, HGPRBMY31

The use of mammalian cell reporter assays to demonstrate functional coupling of known GPCRs (G Protein Coupled Receptors) has been well documented in the literature (Gilman, 1987, Boss et al., 1996; Alam & Cook, 1990; George et al., 1997; Selbie & Hill, 1998; Rees et al., 1999). Reporter assays have been successfully used for identifying novel small molecule agonists or antagonists against GPCRs (Zlokarnik et al., 1998; George et al., 1997; Boss et al., 1996; Rees et al, 2001). In such reporter assays, a promoter is regulated as a direct consequence of activation of specific signal transduction cascades following small molecule binding to a GPCR (Alam & Cook 1990; Selbie & Hill, 1998; Boss et al., 1996; George et al., 1997; Gilman, 1987).

A number of response element-based reporter systems have been developed that enable the study of GPCR function. These include cAMP response element (CRE) reporter genes for G alpha i/o and G alpha s-coupled GPCRs, Nuclear Factor Activator of Transcription (NFAT) reporters for G alpha q/11-coupled receptors and MAP kinase reporter for use in Galpha i/o coupled receptors (Selbie & Hill, 1998; Boss et al., 1996; George et al., 1997; Gilman, 1987; Rees et al., 2001). Transcriptional response elements that regulate the expression of firefly luciferase (PathDetect® in Vivo Signal Transduction Pathway cis-Reporting Systems; Stratagene) have been implemented to characterize the function of the orphan HGPRBMY31 polypeptide of the present invention. The system enables demonstration of constitutive G-protein coupling to endogenous cellular signaling components upon overexpression of orphan receptors. Overexpression has been shown to represent a physiologically relevant event. For example, it has been shown that overexpression occurs in nature during metastatic carcinomas, wherein defective expression of the monocyte chemotactic protein 1 receptor, CCR2, in macrophages is associated with the incidence of human ovarian carcinoma (Sica, et al.,2000; Salcedo et al., 2000). Indeed, it has been shown that overproduction of the Beta 2 Adrenergic Receptor in transgenic mice leads to constitutive activation of the receptor signaling pathway such that these mice exhibit increased cardiac output (Kypson et al., 1999; Dorn et al., 1999). These are only a few of the many examples demonstrating constitutive activation of GPCRs whereby many of these receptors are likely to be in an active conformation (J.Wess 1997).

### Materials and Methods

### DNA Constructs

The putative GPCR HGPRBMY31 cDNA was PCR amplified using Platinum Taq HiFi™ (Invitrogen). The primers used in the PCR reaction were specific to the HGPRBMY31 polynucleotide and were ordered from Genset (5 prime primer: 5′-GGGGACAAGTTTGTACAAAAAAGCAGGCTTCACCATGAACCAGACTTTGA ATAGCAGTG-3′ (SEQ ID NO:41), 3 prime primer: 5′-GGGGACCACTTTGTACAAGAAAGCTGGGTCTCAAGCCCCCATCTCATTG-3′ (SEQ ID NO:42). Both primers contained attB recombination sites for use in the Gateway™ Cloning System (Invitrogen). The product from the PCR reaction was isolated from a 0.8% Agarose gel (FMC) and purified using a Gel Extraction Kit™ from Qiagen.

The purified product was then recombined overnight with the PDONRTm2O1 entry vector from Invitrogen using BP Clonase™ Enzyme Mix (Invitrogen). One microliter of the reaction was subsequently used to transform DH5 alpha cloning efficiency competent E. coli™ (Invitrogen). The plasmid DNA from the Kanamycin resistant clones was isolated on a Biorobot 9600 (Qiagen), and sequenced using standard transposon mediated DNA sequencing techniques. Once the correct sequence of the entry clone was confirmed (e.g., to ensure clone did not contain any PCR introduced base changes that could result in missense mutations), the purified DNA was recombined with the expression vector pEF-DEST5 ₁TM (Invitrogen) using LR Clonase™ Enzyme Mix (Invitrogen). Following overnight incubation at room temperature, one microliter of the reaction was used to transform DH10B™ E. coli (Invitrogen) by electroporation. The plasmid DNA from an ampicillin resistant clone was purified using the Qiagen Midiprep™ plasmid DNA purification kit. A detailed description of the pEF-DEST51™ mammalian expression vector may be found in the Invitrogen manual which is hereby incorporated herein by reference.

### Transient Transfection and Luciferase Detection

The pEF-DEST51™ vector containing the orphan HGPRBMY31 cDNA, and the PathDetect™ CRE-Luciferase reporter plasmid pCRE-Luc (Stratagene) were used to co-transfect CHO-K1 and HEK 293T cells (ATCC) using Lipofectamine™ according to the manufacturers specifications (Invitrogen). Two days later, the cells were lysed and analyzed for Luciferase expression using the Bright-Glo™ Luciferase Assay System (Promega). All cell culture reagents were purchased from Mediatech.

The changes in gene expression as a consequence of constitutive G-protein coupling of the orphan HGPRBMY31 GPCR, can be visualized using Luciferase as a reporter which catalyzes the mono-oxygenation of beetle luciferin into oxyluciferin and in the process emits a photon of light. The lysates of the CHO-K1 and HEK 293T cell lines, transiently transfected with the orphan HGPRBMY31 GPCR and pCRE-Luc, were analyzed using the 1450 MicroBeta JET Liquid Scintillation and Luminescence Counter (Wallac).

### Results—HGPRBMY31 Constitutively Inhibits Gene Expression Through the CRE Response Element

There is strong evidence that certain GPCRs exhibit a cDNA concentration-dependent constitutive activity detectable via cAMP response element (CRE) luciferase reporters (Chen et al., 1999). To demonstrate functional coupling of the putative GPCR HGPRBMY31, the HGPRBMY31 ORF was cloned it into pEF-DEST51™ and transiently transfected into CHO-K1 and HEK293 cells in the presents of pCRE-Luc. Control transfections contained the pEF-DEST51TM vector alone and pCRE-Luc. The cells were analyzed for luminescence with and without stimulation by 10 uM Forskolin (Sigma).

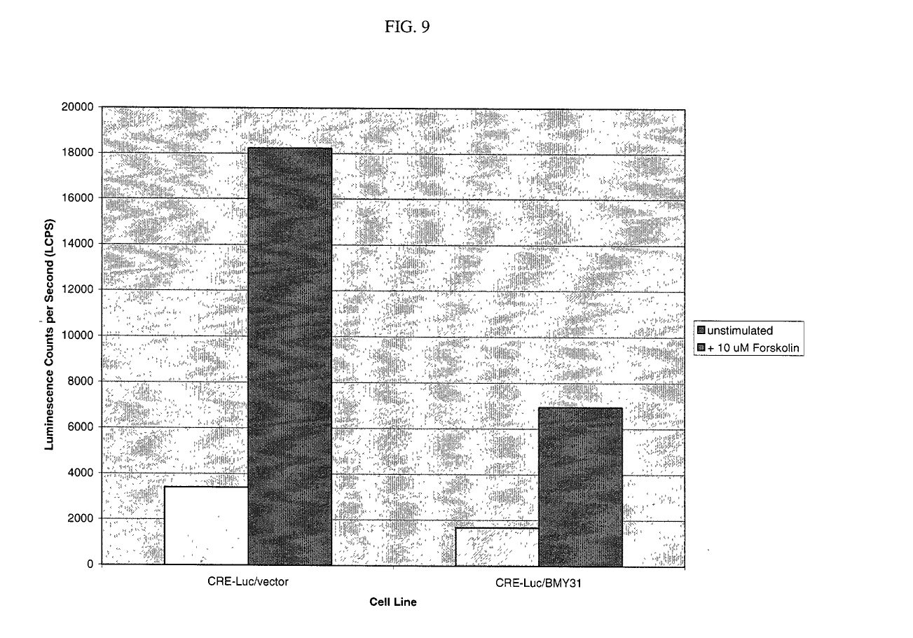

The luminescence data demonstrates the constitutive activity of HGPRBMY31 in CHO-K1 and HEK 293T cell lines as evidenced by the significant decrease in intracellular cAMP levels in HGPRBMY31 cotransfected cell lines compared to cotransfection with the vector alone (see FIGS. 8 and 9). This decrease in cAMP is maintained even after stimulation of the cells with Forskolin. Taken together, these data demonstrate that overexpression of HGPRBMY31 leads to constitutive coupling of signaling to a pathway known to be mediated by G i/o coupled receptors that inhibit CRE response elements.

In conclusion, the results are consistent with HGPRBMY31 representing a functional GPCR analogous to known G i/o coupled receptors. Therefore, constitutive expression of HGPRBMY31 in the CHO-K1 and HEK 293T cell lines leads to CRE inhibition through a decrease of intracellular cAMP as has been demonstrated for other Gi/o linked GPCRs.

In preferred embodiments, the HGPRBMY31 polynucleotides and polypeptides, including agonists, antagonists, and fragments thereof, are useful for modulating intracellular cAMP levels, modulating cAMP sensitive signaling pathways, and modulating CRE element associated signaling pathways.

I. Screening Paradigm

The Luciferase reporter technology provides a clear path for identifying agonists and antagonists of the HGPRBMY31 polypeptide. Cell lines transiently transfected with HGPRBMY31 will provide the opportunity to screen, indirectly, for agonists and antagonists of HGPRBMY31 by looking for small molecules that alter the luciferase response. HGPRBMY31 modulator screens may be carried out using a variety of high throughput methods known in the art, though preferably using a fully automated UHTSS system. HGPRBMY31 transfected cell lines would represent a base line of luciferase expression. Following treatment with 10 uM Forskolin, vector alone transfected cells fully activate the CRE-response element demonstrating the dynamic range of the assay. Screening for antagonists of the HGPRBMY31 induced repression of cAMP would entail looking for molecules that restore luciferase to near that of vector alone transfected cells, either in the presence or absence of adenylate cyclase activatiors such as forskolin.

In preferred embodiments, the HGPRBMY31 transfected CHO-K1 and HEK 293T cell lines of the present invention are useful for the identification of agonists and antagonists of the HGPRBMY31 polypeptide. Representative uses of these cell lines would be their inclusion in a method of identifying HGPRBMY31 agonists and antagonists. Preferably, the cell lines are useful in a method for identifying a compound that modulates the biological activity of the HGPRBMY31 polypeptide, comprising the steps of (a) combining a candidate modulator compound with a host cell expressing the HGPRBMY31 polypeptide having the sequence as set forth in SEQ ID NO:2; and (b) measuring an effect of the candidate modulator compound on the activity of the expressed HGPRBMY31 polypeptide. Representative vectors expressing the HGPRBMY31 polypeptide are referenced herein (e.g., pEF-DEST51™) or otherwise known in the art.

The cell lines are also useful in a method of screening for a compound that is capable of modulating the biological activity of HGPRBMY31 polypeptide, comprising the steps of: (a) determining the biological activity of the HGPRBMY31 polypeptide in the absence of a modulator compound; (b) contacting a host cell expression the HGPRBMY31 polypeptide with the modulator compound; and (c) determining the biological activity of the HGPRBMY31 polypeptide in the presence of the modulator compound; wherein a difference between the activity of the HGPRBMY31 polypeptide in the presence of the modulator compound and in the absence of the modulator compound indicates a modulating effect of the compound. Additional uses for these cell lines are described herein or otherwise known in the art

1. Rees, S., Brown, S., Stables, J.: Reporter gene systems for the study of G Protein Coupled Receptor signalling in mammalian cells. In Milligan G. (ed.): Signal Transduction: A practical approach. Oxford: Oxford University Press, 1999: 171-221.

2. Alam, J., Cook, J. L.: Reporter Genes: Application to the study of mammalian gene transcription. Anal. Biochem. 1990; 188: 245-254.

3. Selbie, L. A. and Hill, S. J.: G protein-coupled receptor cross-talk: The fine-tuning of multiple receptor-signaling pathways. TiPs. 1998; 19: 87-93.

4. Boss, V., Talpade, D. J., and Murphy, T. J.: Induction of NFAT mediated transcription by Gq-coupled Receptors in lympoid and non-lymphoid cells. JBC. 1996; 271: 10429-10432.

5. George, S. E., Bungay, B. J., and Naylor, L. H.: Functional coupling of endogenous serotonin (5-HT1B) and calcitonin (Cla) receptors in Cho cells to a cyclic AMP-responsive luciferase reporter gene. J. Neurochem. 1997; 69: 1278-1285.

6. Suto, C M, Igna D M: Selection of an optimal reporter for cell-based high throughput screening assays. J. Biomol. Screening. 1997; 2: 7-12.

7. Zlokarnik, G., Negulescu, P. A., Knapp, T. E., More, L., Burres, N., Feng, L., Whitney, M., Roemer, K., and Tsien, R. Y. Quantitation of transcription and clonal selection of single living cells with a B-Lactamase Reporter. Science. 1998; 279: 84-88.

8. S. Fiering et. al., Genes Dev. 4, 1823 (1990).

9. J. Karttunen and N. Shastri, PNAS 88, 3972 (1991).

10. Hawes, B. E., Luttrell. L. M., van Biesen, T., and Lefkowitz, R. J. (1996) JBC 271, 12133-12136.

11. Gilman, A. G. (1987) Annul. Rev. Biochem. 56, 615-649.

12. Maniatis et al.,

13. Salcedo, R., Ponce, M. L., Young, H. A., Wasserman, K., Ward, J. M., Kleinman, H. K., Oppenheim, J. J., Murphy, W. J. Human endothelial cells express CCR2 and respond to MCP-1: direct role of MCP-1 in angiogenesis and tumor progression. Blood. 2000; 96 (1): 34-40.

14. Sica, A., Saccani, A., Bottazzi, B., Bernasconi, S., Allavena, P., Gaetano, B., LaRossa, G., Scotton, C., Balkwill F., Mantovani, A. Defective expression of the monocyte chemotactic protein 1 receptor CCR2 in macrophages associated with human ovarian carcinoma. J. Immunology. 2000; 164: 733-8.

15. Kypson, A., Hendrickson, S., Akhter, S., Wilson, K., McDonald, P., Lilly, R., Dolber, P., Glower, D., Lefkowitz, R., Koch, W. Adenovirus-mediated gene transfer of the B2 AR to donor hearts enhances cardiac function. Gene Therapy. 1999; 6: 1298-304.

16. Dorn, G. W., Tepe, N. M., Lorenz, J. N., Kock, W. J., Ligget, S. B. Low and high level transgenic expression of B2AR differentially affect cardiac hypertrophy and function in Galpha q-overexpressing mice. PNAS. 1999; 96: 6400-5.

17. J. Wess. G protein coupled receptor: molecular mechanisms involved in receptor activation and selectivity of G-protein recognition.

18. Whitney, M, Rockenstein, E, Cantin, G., Knapp, T., Zlokarnik, G., Sanders, P., Durick, K., Craig, F. F., and Negulescu, P. A. A genome-wide functional assay of signal transduction in living mammalian cells. 1998. Nature Biotech. 16: 1329-1333.

19. BD Biosciences: FACS Vantage SE Training Manual. Part Number 11-11020-00 Rev. A. August 1999.

20. Chen, G., Jaywickreme, C., Way, J., Armour S., Queen K., Watson., C., Ignar, D., Chen, W. J., Kenakin, T. Constitutive Receptor systems for drug discovery. J. Pharmacol. Toxicol. Methods 1999; 42: 199-206.

### Example 7

### Method of Creating N- and C-Terminal Deletion Mutants Corresponding to the HGPRBMY31 and HGPRBMY31 Variant Polypeptide

As described elsewhere herein, the present invention encompasses the creation of N- and C-terminal deletion mutants, in addition to any combination of N- and C-terminal deletions thereof, corresponding to the HGPRBMY31 and/or HGPRBMY31 variant polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutants of the present invention, exemplary methods are described below.

Briefly, using the isolated cDNA clone encoding the full-length HGPRBMY31 and/or HGPRBMY31 variant polypeptide sequence, appropriate primers of about 15-25 nucleotides derived from the desired 5′ and 3′ positions of SEQ ID NO:1 and/or SEQ ID NO:3 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences (DYKDDDDK (SEQ ID NO:40), kozac sequences, or other sequences discussed and/or referenced herein.

Representative PCR amplification conditions are provided below, although the skilled artisan would appreciate that other conditions may be required for efficient amplification. A 100 microliter PCR reaction mixture may be prepared using 10 ng of the template DNA (cDNA clone of HGPRBMY31), 200 uM 4dNTPs, 1 uM primers, 0.25U Taq DNA polymerase (PE), and standard Taq DNA polymerase buffer. Typical PCR cycling condition are as follows:

20-25 cycles:

45 sec, 93 degrees

2 min, 50 degrees

2 min, 72 degrees

1 cycle:

10 min, 72 degrees

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants may be determined by reference to the following formula:

(S+(X*3)) to ((S+(X*3))+25),

wherein ‘S’ is equal to the nucleotide position of the initiating start codon of the HGPRBMY31 gene (SEQ ID NO:1), and/or the HGPRBMY31 variant gene (SEQ ID NO:3) and ‘X’ is equal to the most N-terminal amino acid of the intended N-terminal deletion mutant. The first term provides the start 5′ nucleotide position of the 5′ primer, while the second term provides the end 3′ nucleotide position of the 5′ primer corresponding to sense strand of SEQ ID NO:1 and/or SEQ ID NO:3. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25),

wherein ‘S’ is equal to the nucleotide position of the initiating start codon of the HGPRBMY31 gene (SEQ ID NO:1) and the HGPRBMY31_variant gene (SEQ ID NO:3), and ‘X’ is equal to the most C-terminal amino acid of the intended N-terminal deletion mutant. The first term provides the start 5′ nucleotide position of the 3′ primer, while the second term provides the end 3′ nucleotide position of the 3′ primer corresponding to the anti-sense strand of SEQ ID NO:1 and/or SEQ ID NO:3. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

In preferred embodiments, the following N-terminal HGPRBMY31 deletion polypeptides are encompassed by the present invention: M1-A307, N2-A307, Q3-A307, T4-A307, L5-A307, N6-A307, S7-A307, S8-A307, G9-A307, T10-A307, V11-A307, E12-A307, S13-A307, A14-A307, L15-A307, N16-A307, Y17-A307, S18-A307, R19-A307, G20-A307, S21-A307, T22-A307, V23-A307, H24-A307, T25-A307, A26-A307, Y27-A307, L28-A307, V29-A307, L30-A307, S31-A307, S32-A307, L33-A307, A34-A307, M35-A307, F36-A307, T37-A307, C38-A307, L39-A307, C40-A307, G41-A307, M42-A307, A43-A307, G44-A307, N45-A307, S46-A307, M47-A307, V48-A307, 149-A307, W50-A307, L51-A307, L52-A307, G53-A307, F54-A307, R55-A307, M56-A307, H57-A307, R58-A307, N59-A307, P60-A307, F61-A307, C62-A307, 163-A307, Y64-A307, 165-A307, L66-A307, N67-A307, L68-A307, A69-A307, A70-A307, A71-A307, D72-A307, L73-A307, L74-A307, F75-A307, L76-A307, F77-A307, S78-A307, M79-A307, A80-A307, S81-A307, T82-A307, L83-A307, S84-A307, L85-A307, E86-A307, T87-A307, Q88-A307, P89-A307, L90-A307, V91-A307, N92-A307, T93-A307, T94-A307, D95-A307, K96-A307, V97-A307, H98-A307, E99-A307, L100-A307, M101-A307, K102-A307, R103-A307, L104-A307, M105-A307, Y106-A307, F107-A307, A108-A307, Y109-A307, T110-A307, V111-A307, G112-A307, L113-A307, S114-A307, L115-A307, L116-A307, T117-A307, A118-A307, 119-A307, S120-A307, T121-A307, Q122-A307, R123-A307, C124-A307, L125-A307, S126-A307, V127-A307, L128-A307, F129-A307, P130-A307, 1131-A307, W132-A307, F133-A307, K134-A307, C135-A307, H136-A307, R137-A307, P138-A307, R139-A307, H140-A307, L141-A307, S142-A307, A143-A307, W144-A307, V145-A307, C146-A307, G147-A307, L148-A307, L149-A307, W150-A307, T151-A307, L152-A307, C153-A307, L154-A307, L155-A307, M156-A307, N157-A307, G158-A307, L159-A307, T160-A307, S161-A307, S162-A307, F163-A307, C164-A307, S165-A307, K166-A307, F167-A307, L168-A307, K169-A307, F170-A307, N171-A307, E172-A307, D173-A307, R174-A307, C175-A307, F176-A307, R177-A307, V178-A307, D179-A307, M180-A307, V181-A307, Q182-A307, A183-A307, A184-A307, L185-A307, 1186-A307, M187-A307, G188-A307, V189-A307, L190-A307, T191-A307, P192-A307, V193-A307, M194-A307, T195-A307, L196-A307, S197-A307, S198-A307, L199-A307, T200-A307, L201-A307, F202-A307, V203-A307, W204-A307, V205-A307, R206-A307, R207-A307, S208-A307, S209-A307, Q210-A307, Q211-A307, W212-A307, R213-A307, R214-A307, Q215-A307, P216-A307, T217-A307, R218-A307, L219-A307, F220-A307, V221-A307, V222-A307, V223-A307, L224-A307, A225-A307, S226-A307, V227-A307, L228-A307, V229-A307, F230-A307, L231-A307, 1232-A307, C233-A307, S234-A307, L235-A307, P236-A307, L237-A307, S238-A307, 1239-A307, Y240-A307, W241-A307, F242-A307, V243-A307, L244-A307, Y245-A307, W246-A307, L247-A307, S248-A307, L249-A307, P250-A307, P251-A307, E252-A307, M253-A307, Q254-A307, V255-A307, L256-A307, C257-A307, F258-A307, S259-A307, L260-A307, S261-A307, R262-A307, L263-A307, S264-A307, S265-A307, S266-A307, V267-A307, S268-A307, S269-A307, S270-A307, A271-A307, N272-A307, P273-A307, A274-A307, T275-A307, R276-A307, S277-A307, L278-A307, G279-A307, T280-A307, V281-A307, L282-A307, Q283-A307, Q284-A307, A285-A307, L286-A307, R287-A307, E288-A307, E289-A307, P290-A307, E291-A307, L292-A307, E293-A307, G294-A307, G295-A307, E296-A307, T297-A307, P298-A307, T299-A307, V300-A307, and/or G301-A307 of SEQ ID NO:2. Polynucleotide sequences encoding these polypeptides are also provided. The present invention also encompasses the use of these N-terminal HGPRBMY31 deletion polypeptides as immunogenic and/or antigenic epitopes as described elsewhere herein.

In preferred embodiments, the following C-terminal HGPRBMY31 deletion polypeptides are encompassed by the present invention: M1-A307, M1-G306, M1-M305, M1-E304, M1-N303, M1-T302, M1-G301, M1-V300, M1-T299, M1-P298, M1-T297, M1-E296, M1-G295, M1-G294, M1-E293, M1-L292, M1-E291, M1-P290, M1-E289, M1-E288, M1-R287, M1-L286, M1-A285, M1-Q284, M1-Q283, M1-L282, M1-V281, M1-T280, M1-G279, M1-L278, M1-S277, M1-R276, M1-T275, M1-A274, M1-P273, M1-N272, M1-A271, M1-S270, M1-S269, M1-S268, M1-V267, M1-S266, M1-S265, M1-S264, M1-L263, M1-R262, M1-S261, M1-L260, M1-S259, M1-F258, M1-C257, M1-L256, M1-V255, M1-Q254, M1-M253, M1-E252, M1-P251, M1-P250, M1-L249, M1-S248, M1-L247, M1-W246, M1-Y245, M1-L244, M1-V243, M1-F242, M1-W241, M1-Y240, M1-1239, M1-S238, M1-L237, M1-P236, M1-L235, M1-S234, M1-C233, M1-1232, M1-L231, M1-F230, M1-V229, M1-L228, M1-V227, M1-S226, M1-A225, M1-L224, M1-V223, M1-V222, M1-V221, M1-F220, M1-L219, M1-R218, M1-T217, M1-P216, M1-Q215, M1-R214, M1-R213, M1-W212, M1-Q211, M1-Q210, M1-S209, M1-S208, M1-R207, M1-R206, M1-V205, M1-W204, M1-V203, M1-F202, M1-L201, M1-T200, M1-L199, M1-S198, M1-S197, M1-L196, M1-T195, M1-M194, M1-V193, M1-P192, M1-T191, M1-L190, M1-V189, M1-G188, M1-M187, M1-1186, M1-L185, M1-A184, M1-A183, M1-Q182, M1-V181, M1-M180, M1-D179, M1-V178, M1-R177, M1-F176, M1-C175, M1-R174, M1-D173, M1-E172, M1-N171, M1-F170, M1-K169, M1-L168, M1-F167, M1-K166, M1-S165, M1-C164, M1-F163, M1-S162, M1-S161, M1-T160, M1-L159, M1-G158, M1-N157, M1-M156, M1-L155, M1-L154, M1-C153, M1-L152, M1-T151, M1-W150, M1-L149, M1-L148, M1-G147, M1-C146, M1-V145, M1-W144, M1-A143, M1-S142, M1-L141, M1-H140, M1-R139, M1-P138, M1-R137, M1-H136, M1-C135, M1-K134, M1-F133, M1-W132, M1-1131, M1-P130, M1-F129, M1-L128, M1-V127, M1-S126, M1-L125, M1-C124, M1-R123, M1-Q122, M1-T121, M1-S120, M1-1119, M1-A118, M1-T117, M1-L116, M1-L115, M1-S114, M1-L113, M1-G112, M1-V111, M1-T110, M1-Y109, M1-A108, M1-F107, M1-Y106, M1-M105, M1-L104, M1-R103, M1-K102, M1-M101, M1-L100, M1-E99, M1-H98, M1-V97, M1-K96, M1-D95, M1-T94, M1-T93, M1-N92, M1-V91, M1-L90, M1-P89, M1-Q88, M1-T87, M1-E86, M1-L85, M1-S84, M1-L83, M1-T82, M1-S81, M1-A80, M1-M79, M1-S78, M1-F77, M1-L76, M1-F75, M1-L74, M1-L73, M1-D72, M1-A71, M1-A70, M1-A69, M1-L68, M1-N67, M1-L66, M1-165, M1-Y64, M1-163, M1-C62, M1-F61, M1-P60, M1-N59, M1-R58, M1-H57, M1-M56, M1-R55, M1-F54, M1-G53, M1-L52, M1-L51, M1-W50, M1-149, M1-V48, M1-M47, M1-S46, M1-N45, M1-G44, M1-A43, M1-M42, M1-G41, M1-C40, M1-L39, M1-C38, M1-T37, M1-F36, M1-M35, M1-A34, M1-L33, M1-S32, M1-S31, M1-L30, M1-V29, M1-L28, M1-Y27, M1-A26, M1-T25, M1-H24, M1-V23, M1-T22, M1-S21, M1-G20, M1-R19, M1-S18, M1-Y17, M1-N16, M1-L15, M1-A14, M1-S13, M1-E12, M1-V11, M1-T10, M1-G9, M1-S8, and/or M1-S7 of SEQ ID NO:2. Polynucleotide sequences encoding these polypeptides are also provided. The present invention also encompasses the use of these C-terminal HGPRBMY31 deletion polypeptides as immunogenic and/or antigenic epitopes as described elsewhere herein.

In preferred embodiments, the following N-terminal HGPRBMY31 variant deletion polypeptides are encompassed by the present invention: M1-A321, N2-A321, Q3-A321, T4-A321, L5-A321, N6-A321, S7-A321, S8-A321, G9-A321, T10-A321, V11-A321, E12-A321, S13-A321, A14-A321, L15-A321, N16-A321, Y17-A321, S18-A321, R19-A321, G20-A321, S21-A321, T22-A321, V23-A321, H24-A321, T25-A321, A26-A321, Y27-A321, L28-A321, V29-A321, L30-A321, S31-A321, S32-A321, L33-A321, A34-A321, M35-A321, F36-A321, T37-A321, C38-A321, L39-A321, C40-A321, G41-A321, M42-A321, A43-A321, G44-A321, N45-A321, S46-A321, M47-A321, V48-A321, 149-A321, W50-A321, L51-A321, L52-A321, G53-A321, F54-A321, R55-A321, M56-A321, H57-A321, R58-A321, N59-A321, P60-A321, F61-A321, C62-A321, 163-A321, Y64-A321, 165-A321, L66-A321, N67-A321, L68-A321, A69-A321, A70-A321, A71-A321, D72-A321, L73-A321, L74-A321, F75-A321, L76-A321, F77-A321, S78-A321, M79-A321, A80-A321, S81-A321, T82-A321, L83-A321, S84-A321, L85-A321, E86-A321, T87-A321, Q88-A321, P89-A321, L90-A321, V91-A321, N92-A321, T93-A321, T94-A321, D95-A321, K96-A321, V97-A321, H98-A321, E99-A321, L100-A321, M101-A321, K102-A321, R103-A321, L104-A321, M105-A321, Y106-A321, F107-A321, A108-A321, Y109-A321, T110-A321, V111-A321, G112-A321, L113-A321, S114-A321, L115-A321, L116-A321, T117-A321, A118-A321, 1119-A321, S120-A321, T121-A321, Q122-A321, R123-A321, C124-A321, L125-A321, S126-A321, V127-A321, L128-A321, F129-A321, P130-A321, 1131-A321, W132-A321, F133-A321, K134-A321, C135-A321, H136-A321, R137-A321, P138-A321, R139-A321, H140-A321, L141-A321, S142-A321, A143-A321, W144-A321, V145-A321, C146-A321, G147-A321, L148-A321, L149-A321, W150-A321, T151-A321, L152-A321, C153-A321, L154-A321, L155-A321, M156-A321, N157-A321, G158-A321, L159-A321, T160-A321, S161-A321, S162-A321, F163-A321, C164-A321, S165-A321, K166-A321, F167-A321, L168-A321, K169-A321, F170-A321, N171-A321, E172-A321, D173-A321, R174-A321, C175-A321, F176-A321, R177-A321, V178-A321, D179-A321, M180-A321, V181-A321, Q182-A321, A183-A321, A184-A321, L185-A321, 1186-A321, M187-A321, G188-A321, V189-A321, L190-A321, T191-A321, P192-A321, V193-A321, M194-A321, T195-A321, L196-A321, S197-A321, S198-A321, L199-A321, T200-A321, L201-A321, F202-A321, V203-A321, W204-A321, V205-A321, R206-A321, R207-A321, S208-A321, S209-A321, Q210-A321, Q211-A321, W212-A321, R213-A321, R214-A321, Q215-A321, P216-A321, T217-A321, R218-A321, L219-A321, F220-A321, V221-A321, V222-A321, V223-A321, L224-A321, A225-A321, S226-A321, V227-A321, L228-A321, V229-A321, F230-A321, L231-A321, 1232-A321, C233-A321, S234-A321, L235-A321, P236-A321, L237-A321, S238-A321, 1239-A321, Y240-A321, W241-A321, F242-A321, V243-A321, L244-A321, Y245-A321, W246-A321, L247-A321, S248-A321, L249-A321, P250-A321, P251-A321, E252-A321, M253-A321, Q254-A321, V255-A321, L256-A321, C257-A321, F258-A321, S259-A321, L260-A321, S261-A321, R262-A321, L263-A321, S264-A321, S265-A321, S266-A321, V267-A321, S268-A321, S269-A321, S270-A321, A271-A321, N272-A321, P273-A321, V274-A321, 1275-A321, Y276-A321, F277-A321, L278-A321, V279-A321, G280-A321, S281-A321, R282-A321, R283-A321, S284-A321, H285-A321, R286-A321, L287-A321, P288-A321, T289-A321, R290-A321, S291-A321, L292-A321, G293-A321, T294-A321, V295-A321, L296-A321, Q297-A321, Q298-A321, A299-A321, L300-A321, R301-A321, E302-A321, E303-A321, P304-A321, E305-A321, L306-A321, E307-A321, G308-A321, G309-A321, E310-A321, T311-A321, P312-A321, T313-A321, V314-A321, and/or G315-A321 of SEQ ID NO:4. Polynucleotide sequences encoding these polypeptides are also provided. The present invention also encompasses the use of these N-terminal HGPRBMY31 variant deletion polypeptides as immunogenic and/or antigenic epitopes as described elsewhere herein.

In preferred embodiments, the following C-terminal HGPRBMY31 variant deletion polypeptides are encompassed by the present invention: M1-A321, M1-G320, M1-M319, M1-E318, M1-N317, M1-T316, M1-G315, M1-V314, M1-T313, M1-P312, M1-T311, M1-E310, M1-G309, M1-G308, M1-E307, M1-L306, M1-E305, M1-P304, M1-E303, M1-E302, M1-R301, M1-L300, M1-A299, M1-Q298, M1-Q297, M1-L296, M1-V295, M1-T294, M1-G293, M1-L292, M1-S291, M1-R290, M1-T289, M1-P288, M1-L287, M1-R286, M1-H285, M1-S284, M1-R283, M1-R282, M1-S281, M1-G280, M1-V279, M1-L278, M1-F277, M1-Y276, M1-1275, M1-V274, M1-P273, M1-N272, M1-A271, M1-S270, M1-S269, M1-S268, M1-V267, M1-S266, M1-S265, M1-S264, M1-L263, M1-R262, M1-S261, M1-L260, M1-S259, M1-F258, M1-C257, M1-L256, M1-V255, M1-Q254, M1-M253, M1-E252, M1-P251, M1-P250, M1-L249, M1-S248, M1-L247, M1-W246, M1-Y245, M1-L244, M1-V243, M1-F242, M1-W241, M1-Y240, MI-1239, M1-S238, M1-L237, M1-P236, M1-L235, M1-S234, M1-C233, M1-1232, M1-L231, M1-F230, M1-V229, M1-L228, M1-V227, M1-S226, M1-A225, M1-L224, M1-V223, M1-V222, M1-V221, M1-F220, M1-L219, M1-R218, M1-T217, M1-P216, M1-Q215, M1-R214, M1-R213, M1-W212, M1-Q211, M1-Q210, M1-S209, M1-S208, M1-R207, M1-R206, M1-V205, M1-W204, M1-V203, M1-F202, M1-L201, M1-T200, M1-L199, M1-S198, M1-S197, M1-L196, M1-T195, M1-M194, M1-V193, M1-P192, M1-T191, M1-L190, M1-V189, M1-G188, M1-M187, M1-1186, M1-LI85, M1-A184, M1-A183, M1-Q182, M1-V181, M1-M180, M1-D179, M1-V178, M1-R177, M1-F176, M1-C175, M1-R174, M1-D173, M1-E172, M1-N171, M1-F170, M1-K169, M1-L168, M1-Fi67, M1-K166, M1-S165, M1-C164, M1-F163, M1-S162, M1-S161, MI-T160, M1-L159, M1-G158, M1-N157, M1-M156, M1-LI55, M1-L154, M1-C153, M1-L152, M1-T151, M1-W150, M1-L149, M1-L148, M1-G147, M1-C146, M1-V145, M1-W144, M1-A143, M1-S142, M1-L141, M1-H140, M1-R139, M1-P138, M1-R137, M1-H136, M1-C135, M1-K134, M1-F133, M1-W132, M1-1131, M1-P130, M1-F129, M1-L128, M1-V127, M1-S126, M1-L125, M1-C124, M1-R123, M1-Q122, M1-T121, M1-S120, M1-1119, M1-A118, M1-T117, M1-L116, M1-L115, M1-S114, M1-L113, M1-G112, MI-V111, M1-T110, M1-Y109, M1-A108, M1-F107, M1-Y106, M1-M105, M1-L104, M1-R103, M1-K102, M1-M101, M1-L100, M1-E99, M1-H98, M1-V97, M1-K96, M1-D95, M1-T94, M1-T93, M1-N92, M1-V91, M1-L90, M1-P89, M1-Q88, M1-T87, M1-E86, M1-L85, M1-S84, M1-L83, M1-T82, M1-S81, M1-A80, M1-M79, M1-S78, M1-F77, M1-L76, M1-F75, M1-L74, M1-L73, M1-D72, M1-A71, M1-A70, M1-A69, M1-L68, M1-N67, M1-L66, M1-165, M1-Y64, M1-163, M1-C62, M1-F61, M1-P60, M1-N59, M1-R58, M1-H57, M1-M56, M1-R55, M1-F54, M1-G53, M1-L52, M1-L51, M1-W50, M1-149, M1-V48, M1-M47, M1-S46, M1-N45, M1-G44, M1-A43, M1-M42, M1-G41, M1-C40, M1-L39, M1-C38, M1-T37, M1-F36, M1-M35, M1-A34, M1-L33, M1-S32, M1-S31, M1-L30, M1-V29, M1-L28, M1-Y27, M1-A26, M1-T25, M1-H24, M1-V23, M1-T22, M1-S21, M1-G20, M1-R19, M1-S18, M1-Y17, M1-N16, M1-L15, M1-A14, M1-S13, M1-E12, M1-V11, M1-T10, M1-G9, M1-S8, and/or M1-S7 of SEQ ID NO:4. Polynucleotide sequences encoding these polypeptides are also provided. The present invention also encompasses the use of these C-terminal HGPRBMY31 variant deletion polypeptides as immunogenic and/or antigenic epitopes as described elsewhere herein.

Alternatively, preferred polypeptides of the present invention may comprise polypeptide sequences corresponding to, for example, internal regions of the HGPRBMY31 and/or HGPRBMY31 variant polypeptide (e.g., any combination of both N- and C-terminal HGPRBMY31 and/or HGPRBMY31 variant polypeptide deletions) of SEQ ID NO:2 and/or SEQ ID NO:4, respectively. For example, internal regions could be defined by the equation: amino acid NX to amino acid CX, wherein NX refers to any N-terminal deletion polypeptide amino acid of HGPRBMY31 (SEQ ID NO:2) and/or HGPRBMY31 variant (SEQ ID NO:4), and where CX refers to any C-terminal deletion polypeptide amino acid of HGPRBMY31 (SEQ ID NO:2) and/or HGPRBMY31 variant (SEQ ID NO:4), respectively. Polynucleotides encoding these polypeptides are also provided. The present invention also encompasses the use of these polypeptides as an immunogenic and/or antigenic epitope as described elsewhere herein.

### Example 8

### Alternative Functional Characterization of HGPRBMY31

The putative GPCR HGPRBMY31 cDNA is PCR amplified using PFU™ (Stratagene). The primers that are used in the PCR reaction are specific to the HGPRBMY31 polynucleotide and are ordered from Gibco BRL, where the 5 prime primer of SEQ ID NO:17 may be used. The 3 prime primer of SEQ ID NO:18 may be used to add a Flag-tag epitope to the HGPRBMY31 polypeptide for immunocytochemistry. Additionally, SEQ ID NO:17 may be modified to include a HindIII site at the 5′ end, such that the resulting primer sequence is SEQ ID NO:19. Further, SEQ ID NO:18 may be modified to include a BamHI site at the 5′ end, and an optional kozak sequence, followed by the complementary encoding sequence of the FLAG tag epitope, followed by the SEQ ID NO:18 sequence to produce a primer of SEQ ID NO:20.

| | |
|---|---|
| HGRBMY31s | |
| 5′-GTCCCCAAGCTTGCACCTCTCGTAGGGATG | (SEQ ID NO:19) |
| AACCAGAC-3′ | |
| HGRBMY31a | |
| 5′-CGGGATCCTACTITGTCGTCGTCGTCCTTGT | (SEQ ID NO:20) |
| AGTCCACGGTCCCACTGCTATTC-3′ | |

The product from the PCR reaction is isolated from a 0.8% Agarose gel (Invitrogen) and purified using a Gel Extraction Kit TM from Qiagen.

The purified product is then digested overnight along with the pcDNA3.1 Hygro™ mammalian expression vector from Invitrogen using the Hindll and BamHI restriction enzymes (N ew England Biolabs). These digested products are then purified using the Gel Extraction Kit™ from Qiagen and subsequently ligated to the pcDNA3.1 Hygro™ expression vector using a DNA molar ratio of 4 parts insert: 1 vector. All DNA modification enzymes are purchased from NEB. The ligation is incubated overnight at 16° C., after which time, one microliter of the mix is used to transform DH5 alpha cloning efficiency competent E. coli™ (Gibco BRL). A detailed description of the pcDNA3.1 Hygro™ mammalian expression vector is available at the Invitrogen web site (www.Invitrogen.com). The plasmid DNA from the ampicillin resistant clones is isolated using the Wizard DNA Miniprep System™ from Promega. Positive clones are then confirmed and scaled up for purification using the Qiagen Maxiprep TM plasmid DNA purification kit.

### Cell Line Generation

The pcDNA3.1hygro vector containing the orphan HGPRBMY31 cDNA is used to transfect CHO-NFAT/CRE or the HEK/CRE (Aurora Biosciences) cells using Lipofectamine 2000™ according to the manufacturers specifications (Gibco BRL). Two days later, the cells are split 1:3 into selective media (DMEM 11056, 600 g/ml Hygromycin, 200 g/ml Zeocin, 10% FBS). All cell culture reagents are purchased from Gibco BRL-Invitrogen.

The CHO-NFAT/CRE or HEK/CRE cell lines, transiently or stably transfected with the orphan HGPRBMY31 GPCR, are analyzed using the FACS Vantage SE TM (BD), fluorescence microscopy (Nikon) and the UL Analyst TM (Molecular Devices). In this system, changes in real-time gene expression, as a consequence of constitutive G-protein coupling of the orphan HGPRBMY31 GPCR, are examined by analyzing the fluorescence emission of the transformed cells at 447 nm and 518 nm. The changes in gene expression are visualized using Beta-Lactamase as a reporter, that, when induced by the appropriate signaling cascade, hydrolyzes an intracellularly loaded, membrane-permeant ester substrate Cephalosporin-Coumarin-Fluorescein2/Acetoxymethyl (CCF2/AM™ Aurora Biosciences; Zlokarnik, et al., 1998). The CCF2/AM™ substrate is a 7-hydroxycoumarin cephalosporin with a fluorescein attached through a stable thioether linkage. Induced expression of the Beta-Lactamase enzyme is readily apparent since each enzyme molecule produced can change the fluorescence of many CCF2/AM™ substrate molecules. A schematic of this cell based system is shown below.

[image]

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only—thus giving rise to blue fluorescent emission at 447 nm.

Fluorescent emissions are detected using a Nikon-TE300 microscope equipped with an excitation filter (D405/IOX-25), dichroic reflector (430DCLP), and a barrier filter for dual DAPI/FITC (510 nM) to visually capture changes in Beta-Lactamase expression. The FACS Vantage SE is equipped with a Coherent Enterprise II Argon Laser and a Coherent 302C Krypton laser. In flow cytometry, UV excitation at 351-364 nm from the Argon Laser or violet excitation at 407 nm from the Krypton laser is used. The optical filters on the FACS Vantage SE are HQ460/50m and HQ535/40m bandpass separated by a 490 dichroic mirror.

Prior to analyzing the fluorescent emissions from the cell lines as described above, the cells are loaded with the CCF2/AM substrate. A 6× CCF2/AM loading buffer is prepared whereby 1 mM CCF2/AM (Aurora Biosciences) is dissolved in 100% DMSO (Sigma). Stock solution (12 μl) is added to 60 μl of 100 mg/ml Pluronic F127 (Sigma) in DMSO containing 0.1% Acetic Acid (Sigma). This solution is added while vortexing to 1 mL of Sort Buffer (PBS minus calcium and magnesium-Gibco-25 mM HEPES-Gibco-pH 7.4, 0.1% BSA). Cells are placed in serum-free media and the 6×CCF2/AM is added to a final concentration of 1×. The cells are then loaded at room temperature for one to two hours, and then subject to fluorescent emission analysis as described herein. Additional details relative to the cell loading methods and/or instrument settings may be found by reference to the following publications: see Zlokarnik, et al., 1998; Whitney et al., 1998; and B D Biosciences,1999.

### Immunocytochemistry

The cell lines transfected and selected for expression of Flag-epitope tagged orphan GPCRs are analyzed by immunocytochemistry. The cells are plated at 1×10³ in each well of a glass slide (VWR). The cells are rinsed with PBS followed by acid fixation for 30 minutes at room temperature using a mixture of 5% Glacial Acetic Acid/90% ethanol. The cells are then blocked in 2% BSA and 0.1% Triton in PBS, and incubated for 2 h at room temperature or overnight at 4C. A monoclonal anti-Flag FITC antibody is diluted at 1:50 in blocking solution and incubated with the cells for 2 h at room temperature. Cells are then washed three times with 0.1% Triton in PBS for five minutes. The slides are overlayed with mounting media dropwise with Biomedia-Gel Mount™ (Biomedia; Containing Anti-Quenching Agent). Cells are examined at 10× magnification using the Nikon TE300 equiped with FITC filter (535 nm).

There is strong evidence that certain GPCRs exhibit a cDNA concentration-dependent constitutive activity through cAMP response element (CRE) luciferase reporters (Chen et al., 1999). In an effort to demonstrate functional coupling of HGPRBMY31 to known GPCR second messenger pathways, the HGPRBMY31 polypeptide are expressed at high constitutive levels in the CHO-NFAT/CRE cell line. To this end, the HGPRBMY31 cDNA is PCR amplified and subcloned into the pcDNA3. 1 hygro™ mammalian expression vector as described herein. Early passage CHO-NFAT/CRE cells are then transfected with the resulting pcDNA3.1 hygro™/HGPRBMY31 construct. Transfected and non-transfected CHO-NFAT/CRE cells (control) are loaded with the CCF2 substrate and stimulated with 10 nM PMA, and 1 M Thapsigargin (NFAT stimulator) or 10 M Forskolin (CRE stimulator) to fully activate the NFAT/CRE element. The cells are then analyzed for fluorescent emission by FACS.

The FACS profile demonstrates the constitutive activity of HGPRBMY31 in the CHO-NFAT/CRE line as may be evidenced by the significant population of cells with blue fluorescent emission at 447 nm. The results indicate that CHO-NFAT/CRE TM cell lines are transfected with the pcDNA3.1 Hygro™/HGPRBMY31 mammalian expression vector. The cells are analyzed via FACS (Fluorescent Assisted Cell Sorter) according to their wavelength emission at 518 nM (Channel R3—Green Cells), and 447 nM (Channel R2—Blue Cells). As may be shown, overexpression of HGPRBMY31 may result in functional coupling and subsequent activation of beta lactamase gene expression, as evidenced by a significant number of cells with fluorescent emission at 447 nM relative to the non-transfected control CHO-NFAT/CRE cells. Control CHO-NFAT/CRE (Nuclear Factor Activator of Transcription (NFAT)/cAMP response element (CRE)) cell lines are those in the absence of the pcDNA3.1 Hygro™/HGPRBMY31 mammalian expression vector transfection. The vast majority of cells emit at 518 nM, with minimal emission observed at 447 nM. The latter is expected since the NFAT/CRE Response Elements remain dormant in the absence of an activated G-protein dependent signal transduction pathway (e.g., pathways mediated by Gq/111 or Gs coupled receptors). As a result, the cell permeant, CCF2/AM™ (Aurora Biosciences; Zlokarnik, et al., 1998) substrate remains intact and emits light at 518 nM.

The NFAT/CRE response element in the untransfected control cell line may not be activated (i.e., beta lactamase not induced), enabling the CCF2 substrate to remain intact, and resulting in the green fluorescent emission at 518 nM. A very low level of leaky Beta Lactamase expression may be detectable as evidenced by a small population of cells emitting at 447 nm. Analysis of a stable pool of cells transfected with HGPRBMY31 may reveal constitutive coupling of the cell population to the NFAT/CRE response element, activation of Beta Lactamase and cleavage of the substrate. The results may demonstrate that overexpression of HGPRBMY31 leads to constitutive coupling of signaling pathways known to be mediated by Gq/11 or Gs coupled receptors that converge to activate either the NFAT or CRE response elements respectively (Boss et al., 1996; Chen et al., 1999).

In preferred embodiments, the HGPRBMY31 polynucleotides and polypeptides, including agonists, antagonists, and fragments thereof, may be useful for modulating intracellular calcium associated signaling pathways.

### Demonstration of Cellular Expression

HGPRBMY31 is tagged at the C-terminus using the Flag epitope and inserted into the pcDNA3.1 hygro™ expression vector, as described herein. Immunocytochemistry of CHO-NFAT/CRE cell lines transfected with the Flag-tagged HGPRBMY31 construct with FITC conjugated Anti Flag monoclonal antibody may demonstrate that HGPRBMY31 is indeed a cell surface receptor. The immunocytochemistry may also confirm expression of the HGPRBMY31 in the CHO-NFAT/CRE cell lines. Briefly, CHO-NFAT/CRE cell lines are transfected with pcDNA3.1 hygro™/HGPRBMY31-Flag vector, fixed with 70% methanol, and permeablized with 0.1% TritonX100. The cells are then blocked with 1% Serum and incubated with a FITC conjugated Anti Flag monoclonal antibody at 1:50 dilution in PBS-Triton. The cells are then washed several times with PBS-Triton, and overlayed with mounting solution. Fluorescent images are captured. The control cell line, non-transfected CHO-NFAT/CRE cell line, may exhibit no detectable fluorescence. These data may provide clear evidence that HGPRBMY31 is expressed in these cells and the majority of the protein is localized to the cell surface. Cell surface localization may be consistent with HGPRBMY31 representing a 7 transmembrane domain containing GPCR. Taken together, the data may indicate that HGPRBMY31 is a cell surface GPCR that can function through increases in Ca²⁺ signal transduction pathways.

### Screening Paradigm

The Aurora Beta-Lactamase technology provides a clear path for identifying agonists and antagonists of the HGPRBMY31 polypeptide. Cell lines that exhibit a range of constitutive coupling activity are identified by sorting through HGPRBMY31 transfected cell lines using the FACS Vantage SE. Several CHO-NFAT/CRE cell lines may be transfected with the pcDNA3.1 Hygro TM/HGPRBMY31 mammalian expression vector isolated via FACS that has either intermediate or high beta lactamase expression levels of constitutive activation, as described herein. Experiments may involve untransfected CHO-NFAT/CRE cells prior to stimulation with 10 nM PMA, 1 M Thapsigargin, and 10 M Forskolin (−P/T/F), CHO-NFAT/CRE cells after stimulation with 10 nM PMA, 1 M Thapsigargin, and 10 M Forskolin (+P/T/F), a representative orphan GPCR (OGPCR) transfected in CHO-NFAT/CRE cells that has an intermediate level of beta lactamase expression, and a representative orphan GPCR transfected in CHO-NFAT/CRE cells that has a high level of beta lactamase expression. For example, cell lines are sorted by those that have an intermediate level of orphan GPCR expression, which may correlate with an intermediate coupling response, using the UL analyst. Such cell lines provide the opportunity to screen, indirectly, for both agonists and antogonists of HGPRBMY31 by searching for inhibitors that block the beta lactamase response, or agonists that increase the beta lactamase response. As described herein, modulating the expression level of beta lactamase directly correlates with the level of cleaved CCF2 substrate. For example, this screening paradigm may work for the identification of modulators of a known GPCR, for example, 5HT6, that couples through adenylate cyclase, in addition to, the identification of modulators of the 5HT2c GPCR, that couples through changes in [Ca²⁺]i. The data may represent cell lines that are engineered with the desired pattern of HGPRBMY31 expression to enable the identification of potent small molecule agonists and antagonists. HGPRBMY31 modulator screens may be carried out using a variety of high throughput methods known in the art, though preferably using the fully automated Aurora UHTSS system. The uninduced, orphan-transfected CHO-NFAT/CRE cell line represents the relative background level of beta lactamase expression. Following treatment with a cocktail of 10 nM PMA, IM Thapsigargin, and 10M Forskolin (P/T/F), the cells may fully activate the CRE-NFAT response element demonstrating the dynamic range of the assay. An orphan transfected CHO-NFAT/CRE cell line that has an intermediate level of beta lactamase expression post P/T/F stimulation and/or an HGPRBMY31 transfected CHO-NFAT/CRE cell line that has a high level of beta lactamase expression post P/T/F stimulation may be observed.

### Example 9

### Signal Transduction Assay

The activity of GPCRs or homologues thereof, can be measured using any assay suitable for the measurement of the activity of a G protein-coupled receptor, as commonly known in the art. Signal transduction activity of a G protein-coupled receptor can be monitor by monitoring intracellular Ca²⁺, cAMP, inosital 1,4,5-trisphophate (IP₃), or 1,2-diacylglycerol (DAG). Assays for the measurement of intracellular Ca⁺ are described in Sakurai et al. (EP 480 381). Intracellular IP₃ can be measured using a kit available from Amersham, Inc. (Arlington Heights, Ill.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the media of the host cells expressing GPCRs. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to free Ca²⁺. The fluorescence can be measured without lysing the cells at an excitation spectrum of 340 nm or 380 nm and at fluorescence spectrum of 500 nm (EP 480 381 to Sakurai et al.).

Upon activation of a G protein-coupled receptor, the rise of free cytosolic Ca²⁺ concentrations is preceded by the hydrolysis of phosphatidylinositol 4,5-bisphosphate. Hydrolysis of this phospholipid by the phospholipase C yields 1,2-diacylglycerol (DAG), which remains in the membrane, and water-soluble inosital 1,4,5-trisphophate (IP3). Binding of ligands or agonists will increase the concentration of DAG and IP₃. Thus, signal transduction activity can be measured by monitoring the concentration of these hydrolysis products.

To measure the IP concentrations, radioactivity labeled ³H-inositol is added to the media of host cells expressing GPCRs. The ³H-inositol is taken up by the cells and incorporated into IP₃. The resulting inositol triphosphate is separated from the mono and di-phosphate forms and measured (EP 480 381 Sakurai et al.). Alternatively, Amersham provides an inosital 1,4,5-triphosphate assay system. With this system Amersham provides tritylated inositol 1,4,5-triphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described in Gilman et al., Proc. Natl. Acad. Sci. 67:305-312 (1970). In addition, a kit for assaying levels of cAMP is available from Diagnostic Products Corp. (Los Angeles, Calif.).

### Example 10

### GPCR Activity

This example describes another method for screening compounds which are GPCR antagonists, and thus inhibit the activation or function of the GPCR polypeptides of the present invention. The method involves determining inhibition of binding of a labeled ligand, such as dATP, dAMP, or UTP, to cells expressing a novel GPCR on the cell surface, or to cell membranes containing the GPCR.

Such a method further involves transfecting a eukaryotic cell with DNA encoding a GPCR polypeptide such that the cell expresses the receptor on its surface. The cell is then contacted with a potential antagonist in the presence of a labeled form of a ligand, such as dATP, dAMP, or UTP. The ligand can be labeled, for example, by radioactivity, fluorescence, chemiluminescence, or any other suitable detectable label commonly known in the art. The amount of labeled ligand bound to the expressed GPCR receptors is measured, for example, by measuring radioactivity associated with transfected cells, or membranes from these cells. If the compound binds to the expressed GPCR, the binding of labeled ligand to the receptor is inhibited, as determined by a reduction of labeled ligand which also binds to the GPCR. This method is called a binding assay. The above-described technique can also be used to determine binding of GPCR agonists.

In a further screening procedure, manmalian cells, for example, but not limited to, CHO, HEK 293, Xenopus oocytes, RBL-2H3, etc., which are transfected with nucleic acid encoding a novel GPCR, are used to express the receptor of interest. The cells are loaded with an indicator dye that produces a fluorescent signal when bound to calcium, and the cells are contacted with a test substance and a receptor agonist, such as dATP, dAMP, or UTP. Any change in fluorescent signal is measured over a defined period of time using, for example, a fluorescence spectrophotometer or a fluorescence imaging plate reader. A change in the fluorescence signal pattern generated by the ligand relative to control indicates that a compound is a potential antagonist or agonist for the receptor.

In yet another screening procedure, manmalian cells are transfected with a GPCR-encoding polynucleotide sequence so as to express the GPCR of interest. The same cells are also transfected with a reporter gene construct that is coupled to/associated with activation of the receptor. Nonlimiting examples of suitable reporter gene systems include luciferase or beta-galactosidase regulated by an appropriate promoter. The engineered cells are contacted with a test substance or compound and a receptor ligand, such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for determining gpcr antagonists or agonists involves introducing ma encoding the gpcr polypeptide into cells (e.g., CHO, HEK 293, RBL-2H3 cells, and the like) in which the receptor is transiently or stably expressed. The receptor cells are then contacted with a ligand for the GPCR, such as DATP, DAMP, or UTP, and a compound to be screened. inhibition or activation of the receptor is then determined by detection of a signal, such as, camp, calcium, proton, or other ions.

### Example 11

### Method of Enhancing the Biological Activity or Functional Characteristics Through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, Del., et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (W P C, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments—further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it may be subjected to Dnase I digestion. About 2-4 μg of the DNA substrate(s) may be digested with 0.0015 units of Dnase I (Sigma) per microliter (μl) in 100 μl of 50 mM Tris-HCL, pH 7.4/1 mM MgCl₂ for 10-20 min. at room temperature. The resulting fragments of 10-50 bp may then be purified by running them through a 2% low-melting point agarose gel by electrophoresis onto DE81 ion-exchange paper (Whatmann) or may be purified using Microcon concentrators (Amicon) of the appropriate molecular weight cutoff, or may use oligonucleotide purification columns (Qiagen), in addition to other methods known in the art. If using DE81 ion-exchange paper, the 10-50 bp fragments may be eluted from said paper using IM NaCl followed by ethanol precipitation.

The resulting purified fragments may then be subjected to a PCR assembly reaction by resuspension in a PCR mixture containing: 2 mM of each dNTP, 2.2 mM MgCl2, 50 mM KCl, 10 mM TrisHCL, pH 9.0, and 0.1% Triton X-100, at a final fragment concentration of 10-30 ng/ul. No primers are added at this point. Taq DNA polymerase (Promega) would be used at 2.5 units per 100 μl of reaction mixture. A PCR program of 94 C for 60s; 94 C for 30s, 50-55 C for 30s, and 72 C for 30s using 30-45 cycles, followed by 72 C for 5 minutes using an MJ Research (Cambridge, Mass.) PTC-150 thermocycler. After the assembly reaction is completed, a 1:40 dilution of the resulting primeness product may then be introduced into a PCR mixture (using the same buffer mixture used for the assembly reaction) containing 0.8 um of each primer and subjecting this mixture to 15 cycles of PCR (using 94 C for 30s, 50 C for 30s, and 72 C for 30s). The referred primers may be primers corresponding to the nucleic acid sequences of the polynucleotide(s) utilized in the shuffling reaction. Said primers may consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6): 1307-1308, (1997)).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16,000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 12

### Phage Display Methods for Identifying Peptide Ligands or Modulators of Orphan GPCRs

### Creation of Peptide Libraries

One of two types of libraries may be created: A 15 mer library for finding peptides that may function as (ant-)agonists, and a 40 mer library for database searches for finding natural ligands.

The 15 mer library may be an aliquot of the 15 mer library originally constructed by GP Smith (Scott, JK and Smith, GP. 1990, Science 249, 386-390). Such a library may be made essentially as described therein.

The 40 mer library may be made essentially as described in Gene, 128, 1993, 59-65: An M13 phage library displaying random 38-amino acid peptides as a source of novel sequences with affinity to selected targets (B K Kay, N B Adey, Y-S He, J P Manfredi, A H Mataragnon, D M Fowlkes), with the exception that a 15 bp complementary region is used to anneal the two oligos, as opposed to 6, 9, or 12 bp, as described below.

| | |
|---|---|
| The oligos used are: Oligo 1: | |
| 5′-CGAAGCGTAAGGGCCCAGCCGGCCNNBNNBNNBNNBNNBNNBNNBNNBN | (SEQ ID NO:30) |
| NBNNBNNBNNBNNBNNBNNBNNBNNBNNBNNBNNBCCGGGTCCGGGCGG-3′ | |
| and | |
| Oligo2: | |
| 5′-AAAAGGAAAAAAGCGGCCGCVNNVNNVNNVNNVNNVNNVNNVNNVNNV | (SEQ ID NO:31) |
| NNVNNVNNVNNVNNVNNVNNVNNVNNVNNVNNGCCGCCCGGACCCGG-3′, | |
| where N = A, G, C, or T; B = C, G, or T; and V = C, A, or G. | |

The oligos are annealed via their 15 base pair complimentary sequences which encode a constant ProGlyProGlyGly pentapeptide sequence between the random 20aa segments, and then extended by standard procedure using Klenow enzyme. This is followed by endonuclease digestion using Sfi1 and Not1 enzymes and ligation to Sfi1 and Not1 cleaved pCantab5E (Pharmacia). The ligation mixture is electroporated into E. coli XL1Blue and generation of phage clones essentially as suggested by Pharmacia for making ScFv antibody libraries in pCantab5E.

### Sequencing of Bound Phage

Standard procedure. Phage in eluates are infected into E. coli host strain (TG1 for 15 mer library; XL1Blue for 40 mer library) and are plated for single colonies. Colonies are grown in liquid and sequenced by standard procedure which involve 1.) generating PCR product with suitable primers of the library segments in the phage genome (15 mer library) or pCantab5E (40 mer library) and 2.) sequencing of the PCR products using one primer of each PCR primer pair. Sequences are visually inspected or by using the Vector NTI alignment tool.

I. Peptide Synthesis

Peptides are synthesized on Fmoc-Knorr amide resin [N-(9-fluorenyl)methoxycarbonyl-Knorr amide-resin, Midwest Biotech, Fishers, Ind.] with an Applied Biosystems (Foster City, Calif.) model 433A synthesizer and theFastMoc chemistry protocol (0.25 mmol scale) supplied with the instrument.

Amino acids are double coupled as their N-alpha-Fmoc-derivatives and reactive side chains are protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group is removed by the multi-step treatment with piperidine in N-Methylpyrrolidone described by the manufacturer. The N-terminal free amines are then treated with 10% acetic anhydride, 5% Diisopropylamine in N-Methylpyrrolidone to yield the N-acetyl-derivative. The protected peptidyl-resins are simultaneously deprotected and removed from the resin by standard methods. The lyophilized peptides are purified on C₁₈ to apparent homogeneity as judged by RP-HPLC analysis. Predicted peptide molecular weights are verified by electrospray mass spectrometry. (J. Biol. Chem . . . vol. 273, pp.12041-12046, 1998)

Cyclic analogs are prepared from the crude linear products. The cystine disulfide may be formed using one of the following methods:

Method 1: A sample of the crude peptide is dissolved in water at a concentration of 0.5 mg/mL and the pH adjusted to 8.5 with NH₄OH. The reaction is stirred, open to room air, and monitored by RP-HPLC.

Once complete, the reaction is brought to pH 4 with acetic acid and lyophilized. The product is purified and characterized as above.

Method 2: A sample of the crude peptide is dissolved at a concentration of 0.5 mg/mL in 5% acetic acid. The pH is adjusted to 6.0 with NH₄OH. DMSO (20% by volume) is added and the reaction is stirred overnight. After analytical RP-HPLC analysis, the reaction is diluted with H20 and triple lyophilized to remove DMSO. The crude product is purified by preparative RP-HPLC. (JACS. vol. 113, 6657, 1991)

### Assessing Affect of Peptides on GPCR Function

The effect of any one of these peptides on the function of the GPCR of the present invention may be determined by adding an effective amount of each peptide to each functional assay. Representative functional assays are described more specifically herein.

### Uses of the Peptide Modulators of the Present Invention

The aforementioned peptides of the present invention are useful for a variety of purposes, though most notably for modulating the function of the GPCR of the present invention, and potentially with other GPCRs of the same G-protein coupled receptor subclass (e.g., peptide receptors, adrenergic receptors, purinergic receptors, etc.), and/or other subclasses known in the art. For example, the peptide modulators of the present invention may be useful as HGPRBMY31 agonists. Alternatively, the peptide modulators of the present invention may be useful as HGPRBMY31 antagonists of the present invention. In addition, the peptide modulators of the present invention may be useful as competitive inhibitors of the HGPRBMY31 cognate ligand(s), or may be useful as non-competitive inhibitors of the HGPRBMY31 cognate ligand(s).

Furthermore, the peptide modulators of the present invention may be useful in assays designed to either deorphan the HGPRBMY31 polypeptide of the present invention, or to identify other agonists or antagonists of the HGPRBMY31 polypeptide of the present invention, particularly small molecule modulators.

### Example 13

### Production of an Antibody from a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### i. Example 34

### Production of an Antibody

a) Hybridoma Technology

The antibodies of the present invention can be prepared by a variety of methods. (See, Current Protocols, Chapter 2.) As one example of such methods, cells expressing HLRRBM1 are administered to an animal to induce the production of sera containing polyclonal antibodies. In a preferred method, a preparation of HLRRBM1 protein is prepared and purified to render it substantially free of natural contaminants. Such a preparation is then introduced into an animal in order to produce polyclonal antisera of greater specific activity.

Monoclonal antibodies specific for protein HLRRBM1 are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (preferably a mouse) is immunized with HLRRBMI polypeptide or, more preferably, with a secreted HLRRBM1 polypeptide-expressing cell. Such polypeptide-expressing cells are cultured in any suitable tissue culture medium, preferably in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the HLRRBM1 polypeptide.

Alternatively, additional antibodies capable of binding to HLRRBM 1 polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the HLRRBM1 protein-specific antibody can be blocked by HLRRBM1. Such antibodies comprise anti-idiotypic antibodies to the HLRRBM1 protein-specific antibody and are used to immunize an animal to induce formation of further HLRRBM1 protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation of Antibody Fragments Directed Against HLRRBM1 From a Library of scFvs

Naturally occurring V-genes isolated from human PBLs are constructed into a library of antibody fragments which contain reactivities against HLRRBMI to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to inoculate 50 ml of 2×TY-AMP-GLU, 2×10⁸ TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 14

### Method of Screening, In Vitro, Compounds That Bind to the HGPRBMY31 Polypeptide

In vitro systems can be designed to identify compounds capable of binding the HGPRBMY31 polypeptide of the invention. Compounds identified can be useful, for example, in modulating the activity of wild type and/or mutant HGPRBMY31 polypeptide, preferably mutant HGPRBMY31 polypeptide, can be useful in elaborating the biological function of the HGPRBMY31 polypeptide, can be utilized in screens for identifying compounds that disrupt normal HGPRBMY31 polypeptide interactions, or can in themselves disrupt such interactions.

The principle of the assays used to identify compounds that bind to the HGPRBMY31 polypeptide involves preparing a reaction mixture of the HGPRBMY31 polypeptide and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring HGPRBMY31 polypeptide or the test substance onto a solid phase and detecting HGPRBMY31 polypeptide/test compound complexes anchored on the solid phase at the end of the reaction. In one embodiment of such a method, the HGPRBMY31 polypeptide can be anchored onto a solid surface, and the test compound, which is not anchored, can be labeled, either directly or indirectly.

In practice, microtitre plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for HGPRBMY31 polypeptide or the test compound to anchor any complexes formed in solution, and a labeled antibody specific for the other component of the possible complex to detect anchored complexes.

Another example of a screening assay to identify compounds that bind to HGPRBMY31, relates to the application of a cell membrane-based scintillation proximity assay (“SPA”). Such an assay would require the idenification of a ligand for HGPRBMY31 polypeptide. Once identified, unlabeled ligand is added to assay-ready plates that would serve as a positive control. The SPA beads and membranes are added next, and then 1²⁵I-labeled ligand is added. After an equilibration period of 2-4 hours at room temperature, the plates can be counted in a scintillation counting machine, and the percent inhibition or stimulation calculated. Such an SPA assay may be based upon a manual, automated, or semi-automated platform, and encompass 96, 384, 1536-well plates or more. Any number of SPA beads may be used as applicable to each assay. Examples of SPA beads include, for example, Leadseeker WGA PS (Amersham cat #RPNQ 0260), and SPA Beads (PVT-PEI-WGA-TypeA; Amersham cat #RPNQ0003). The utilized membranes may also be derived from a number of cell line and tissue sources depending upon the expression profile of the respective polypeptide and the adaptability of such a cell line or tissue source to the development of a SPA-based assay. Examples of membrane preparations include, for example, cell lines transformed to express the receptor to be assayed in CHO cells or HEK cells, for example. SPA-based assays are well known in the art and are encompassed by the present invention. One such assay is described in U.S. Pat. No. 4,568,649, which is incorporated herein by reference. The skilled artisan would acknowledge that certain modifications of known SPA assays may be required to adapt such assays to each respective polypeptide.

One such screening procedure involves the use of melanophores which are transfected to express the HGPRBMY31 polypeptide of the present invention. Such a screening technique is described in PCT WO 92/01810, published Feb. 6, 1992. Such an assay may be employed to screen for a compound which inhibits activation of the receptor polypeptide of the present invention by contacting the melanophore cells which encode the receptor with both the receptor ligand, such as LPA, and a compound to be screened. Inhibition of the signal generated by the ligand indicates that a compound is a potential antagonist for the receptor, i.e., inhibits activation of the receptor.

The technique may also be employed for screening of compounds which activate the receptor by contacting such cells with compounds to be screened and determining whether such compound generates a signal, i.e., activates the receptor. Other screening techniques include the use of cells which express the HGPRBMY31 polypeptide (for example, transfected CHO cells) in a system which measures extracellular pH changes caused by receptor activation. In this technique, compounds may be contacted with cells expressing the receptor polypeptide of the present invention. A second messenger response, e.g., signal transduction or pH changes, is then measured to determine whether the potential compound activates or inhibits the receptor.

Another screening technique involves expressing the HGPRBMY31 polypeptide in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists or agonists by determining inhibition of binding of labeled ligand, such as LPA, to cells which have the receptor on the surface thereof, or cell membranes containing the receptor. Such a method involves transfecting a cell (such as eukaryotic cell) with DNA encoding the HGPRBMY31 polypeptide such that the cell expresses the receptor on its surface. The cell is then contacted with a potential antagonist or agonist in the presence of a labeled form of a ligand, such as LPA. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called binding assay.

Another screening procedure involves the use of mammalian cells (CHO, HEK 293, Xenopus Oocytes, RBL-2H3, etc) which are transfected to express the receptor of interest. The cells are loaded with an indicator dye that produces a fluorescent signal when bound to calcium, and the cells are contacted with a test substance and a receptor agonist, such as LPA. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening procedure involves use of mammalian cells (CHO, HEK293, Xenopus Oocytes, RBL-2H3, etc.) which are transfected to express the receptor of interest, and which are also transfected with a reporter gene construct that is coupled to activation of the receptor (for example, luciferase or beta-galactosidase behind an appropriate promoter). The cells are contacted with a test substance and the receptor agonist (ligand), such as LPA, and the signal produced by the reporter gene is measured after a defined period of time. The signal can be measured using a luminometer, spectrophotometer, fluorimeter, or other such instrument appropriate for the specific reporter construct used. Change of the signal generated by the ligand indicates that a compound is a potential antagonist or agonist for the receptor.

Another screening technique for antagonists or agonits involves introducing RNA encoding the HGPRBMY31 polypeptide into Xenopus oocytes (or CHO, HEK 293, RBL-2H3, etc.) to transiently or stably express the receptor. The receptor oocytes are then contacted with the receptor ligand, such as LPA, and a compound to be screened. Inhibition or activation of the receptor is then determined by detection of a signal, such as, cAMP, calcium, proton, or other ions.

Another method involves screening for HGPRBMY31 polypeptide inhibitors by determining inhibition or stimulation of HGPRBMY31 polypeptide-mediated cAMP and/or adenylate cyclase accumulation or dimunition. Such a method involves transiently or stably transfecting a eukaryotic cell with HGPRBMY31 polypeptide receptor to express the receptor on the cell surface.

The cell is then exposed to potential antagonists or agonists in the presence of HGPRBMY31 polypeptide ligand, such as LPA. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist or agonist binds the receptor, and thus inhibits HGPRBMY31 polypeptide-ligand binding, the levels of HGPRBMY31 polypeptide-mediated cAMP, or adenylate cyclase activity, will be reduced or increased.

One preferred screening method involves co-transfecting HEK-293 cells with a mammalian expression plasmid encoding a G-protein coupled receptor (GPCR), such as HGPRBMY31, along with a mixture comprised of mammalian expression plasmids cDNAs encoding GU15 (Wilkie T. M. et al Proc Natl Acad Sci USA 1991 88: 10049-10053), GU16 (Amatruda T. T. et al Proc Natl Acad Sci USA 1991 8: 5587-5591, and three chimeric G-proteins refered to as Gqi5, Gqs5, and Gqo5 (Conklin B R et al Nature 1993 363: 274-276, Conklin B. R. et al Mol Pharmacol 1996 50: 885-890). Following a 24h incubation the trasfected HEK-293 cells are plated into poly-D-lysine coated 96 well black/clear plates (Becton Dickinson, Bedford, Mass.).

The cells are assayed on FLIPR (Fluorescent Imaging Plate Reader, Molecular Devices, Sunnyvale, Calif.) for a calcium mobilization response following addition of test ligands. Upon identification of a ligand which stimulates calcium mobilization in HEK-293 cells expressing a given GPCR and the G-protein mixtures, subsequent experiments are performed to determine which, if any, G-protein is required for the functional response. HEK-293 cells are then transfected with the test GPCR, or co-transfected with the test GPCR and G015, GD16, GqiS, Gqs5, or Gqo5. If the GPCR requires the presence of one of the G-proteins for functional expression in HEK-293 cells, all subsequent experiments are performed with HEK-293 cell cotransfected with the GPCR and the G-protein which gives the best response. Alternatively, the receptor can be expressed in a different cell line, for example RBL-2H3, without additional Gproteins.

Another screening method for agonists and antagonists relies on the endogenous pheromone response pathway in the yeast, Saccharomyces cerevisiae. Heterothallic strains of yeast can exist in two mitotically stable haploid mating types, MATa and MATa. Each cell type secretes a small peptide hormone that binds to a G-protein coupled receptor on opposite mating type cells which triggers a MAP kinase cascade leading to G1 arrest as a prelude to cell fusion.

Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U.S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclindependent kinases leading to cell cycle arrest; and (iii) construction of reporter genes fused to the FUS 1 gene promoter (where FUS 1 encodes a membrane-anchored glycoprotein required for cell fusion). Downstream reporter genes can permit either a positive growth selection (e.g., histidine prototrophy using the FUS1-HIS3 reporter), or a colorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, J. R. and Thorner, J., Nature 384: 14-16, 1996; Manfredi et al., Mol. Cell. Biol. 16: 4700-4709,1996). This provides a rapid direct growth selection (e.g, using the FUS 1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands.

Functional agonists of sufficient potency (whether natural or surrogate) can be used as screening tools in yeast cell-based assays for identifying G-protein coupled receptor antagonists. For example, agonists will promote growth of a cell with FUS-HIS3 reporter or give positive readout for a cell with FUSI-LacZ. However, a candidate compound which inhibits growth or negates the positive readout induced by an agonist is an antagonist. For this purpose, the yeast system offers advantages over mammalian expression systems due to its ease of utility and null receptor background (lack of endogenous G-protein coupled receptors) which often interferes with the ability to identify agonists or antagonists.

### Example 15

### Bacterial Expression of a Polypeptide

A polynucleotide encoding a polypeptide of the present invention is amplified using PCR oligonucleotide primers corresponding to the 5′ and 3′ ends of the DNA sequence to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 16

### Purification of a Polypeptide From an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000 xg for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000 ×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 17

### Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in Example 15, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described in Example 15. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and Sug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 18

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CVI, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem . . . 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### Example 19

### Assays Detecting Stimulation or Inhibition of B Cell Proliferation and Differentiation

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL10, 1L-13, IL-14 and IL-15. Interestingly, these signals are by themselves weak effectors but can, in combination with various co-stimulatory proteins, induce activation, proliferation, differentiation, homing, tolerance and death among B cell populations.

One of the best studied classes of B-cell co-stimulatory proteins is the TNF-superfamily. Within this family CD40, CD27, and CD30 along with their respective ligands CD154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In Vitro Assay-Purified polypeptides of the invention, or truncated forms thereof, is assessed for its ability to induce activation, proliferation, differentiation or inhibition and/or death in B-cell populations and their precursors. The activity of the polypeptides of the invention on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 105 B-cells suspended in culture medium (RPMI 1640 containing 10% FBS, 5×10-SM 2ME, 100U/ml penicillin, 10 ug/ml streptomycin, and 10-5 dilution of SAC) in a total volume of 150 ul. Proliferation or inhibition is quantitated by a 20h pulse (1 uCi/well) with 3H-thymidine (6.7 Ci/mM) beginning 72 h post factor addition. The positive and negative controls are IL2 and medium respectively.

In Vivo Assay-BALB/c mice are injected (i.p.) twice per day with buffer only, or 2 mg/Kg of a polypeptide of the invention, or truncated forms thereof. Mice receive this treatment for 4 consecutive days, at which time they are sacrificed and various tissues and serum collected for analyses. Comparison of H&E sections from normal spleens and spleens treated with polypeptides of the invention identify the results of the activity of the polypeptides on spleen cells, such as the diffusion of periarterial lymphatic sheaths, and/or significant increases in the nucleated cellularity of the red pulp regions, which may indicate the activation of the differentiation and proliferation of B-cell populations. Immunohistochemical studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from mice treated with polypeptide is used to indicate whether the polypeptide specifically increases the proportion of ThB+, CD45R(B220)dull B cells over that which is observed in control mice.

Likewise, a predicted consequence of increased mature B-cell representation in vivo is a relative increase in serum Ig titers. Accordingly, serum IgM and IgA levels are compared between buffer and polypeptide-treated mice.

One skilled in the art could easily modify the exemplified studies to test the activity of polynucleotides of the invention (e.g., gene therapy), agonists, and/or antagonists of polynucleotides or polypeptides of the invention.

### Example 20

### T Cell Proliferation Assay

A CD3-induced proliferation assay is performed on PBMCs and is measured by the uptake of 3H-thymidine. The assay is performed as follows. Ninety-six well plates are coated with 100 (1/well of mAb to CD3 (HIT3a, Pharmingen) or isotype-matched control mAb (B33.1) overnight at 4 degrees C. (1 (g/ml in 0.05M bicarbonate buffer, pH 9.5), then washed three times with PBS. PBMC are isolated by F/H gradient centrifugation from human peripheral blood and added to quadruplicate wells (5×10⁴/well) of mAb coated plates in RPMI containing 10% FCS and P/S in the presence of varying concentrations of polypeptides of the invention (total volume 200 ul). Relevant protein buffer and medium alone are controls. After 48 hr. culture at 37 degrees C., plates are spun for 2 min. at 1000 rpm and 100 (1 of supernatant is removed and stored −20 degrees C. for measurement of IL-2 (or other cytokines) if effect on proliferation is observed. Wells are supplemented with 100 ul of medium containing 0.5 uCi of 3H-thymidine and cultured at 37 degrees C. for 18-24 hr. Wells are harvested and incorporation of 3H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of polypeptides of the invention.

One skilled in the art could easily modify the exemplified studies to test the activity of polynucleotides of the invention (e.g., gene therapy), agonists, and/or antagonists of polynucleotides or polypeptides of the invention.

### Example 21

### Effect of Polypeptides of the Invention on the Expression of MHC CLASS II, Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and MHC class II antigens). Treatment with activating factors, such as TNF-, causes a rapid change in surface phenotype (increased expression of MHC class I and II, costimulatory and adhesion molecules, downregulation of FC(RII, upregulation of CD83). These changes correlate with increased antigen-presenting capacity and with functional maturation of the dendritic cells.

FACS analysis of surface antigens is performed as follows. Cells are treated 1-3 days with increasing concentrations of polypeptides of the invention or LPS (positive control), washed with PBS containing 1% BSA and 0.02 mM sodium azide, and then incubated with 1:20 dilution of appropriate FITC- or PE-labeled monoclonal antibodies for 30 minutes at 4 degrees C. After an additional wash, the labeled cells are analyzed by flow cytometry on a FACScan (Becton Dickinson).

Effect on the production of cytokines. Cytokines generated by dendritic cells, in particular 1L-12, are important in the initiation of T-cell dependent immune responses. IL-12 strongly influences the development of Th1 helper T-cell immune response, and induces cytotoxic T and NK cell function. An ELISA is used to measure the IL-12 release as follows. Dendritic cells (106/ml) are treated with increasing concentrations of polypeptides of the invention for 24 hours. LPS (100 ng/ml) is added to the cell culture as positive control. Supernatants from the cell cultures are then collected and analyzed for IL-12 content using commercial ELISA kit(e.g., R & D Systems (Minneapolis, Minn.)). The standard protocols provided with the kits are used.

Effect on the expression of MHC Class II, costimulatory and adhesion molecules. Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of polypeptides of the invention or LPS (positive control), washed with PBS containing 1% BSA and 0.02 mM sodium azide, and then incubated with 1:20 dilution of appropriate FITC- or PE-labeled monoclonal antibodies for 30 minutes at 4 degrees C. After an additional wash, the labeled cells are analyzed by flow cytometry on a FACScan (Becton Dickinson).

Monocyte activation and/or increased survival. Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. Polypeptides, agonists, or antagonists of the invention can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×10⁶/ml in PBS containing PI at a final concentration of 5 (g/ml, and then incubated at room temperature for 5 minutes before FACScan analysis. PI uptake has been demonstrated to correlate with DNA fragmentation in this experimental paradigm.

Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×105 cells/ml with increasing concentrations of the a polypeptide of the invention and under the same conditions, but in the absence of the polypeptide. For IL-12 production, the cells are primed overnight with IFN (100 U/ml) in presence of a polypeptide of the invention. LPS (10 ng/ml) is then added. Conditioned media are collected after 24 h and kept frozen until use. Measurement of TNF-alpha, IL-10, MCP-1 and IL-8 is then performed using a commercially available ELISA kit(e.g., R & D Systems (Minneapolis, Minn.)) and applying the standard protocols provided with the kit.

Oxidative burst. Purified monocytes are plated in 96-w plate at 2-1×105 cell/well. Increasing concentrations of polypeptides of the invention are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 nM PMA). The plates are incubated at 37(C for 2 hours and the reaction is stopped by adding 20 μl 1N NaOH per well. The absorbance is read at 610 nm. To calculate the amount of H202 produced by the macrophages, a standard curve of a H2O2 solution of known molarity is performed for each experiment.

One skilled in the art could easily modify the exemplified studies to test the activity of polynucleotides of the invention (e.g., gene therapy), agonists, and/or antagonists of polynucleotides or polypeptides of the invention.

### Example 22

### Biological Effects of HGPRBMY39 Polypeptides of the Invention

### Astrocyte and Neuronal Assays

Recombinant polypeptides of the invention, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate a polypeptide of the invention's activity on these cells.

Moreover, previous reports describing the biological effects of FGF-2 (basic FGF) on cortical or hippocampal neurons in vitro have demonstrated increases in both neuron survival and neurite outgrowth (Walicke et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of a polypeptide of the invention to induce neurite outgrowth can be compared to the response achieved with FGF-2 using, for example, a thymidine incorporation assay.

### Fibroblast and Endothelial Cell Assays

Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE2 assays, the human lung fibroblasts are cultured at 5,000 cells/well in a 96-well plate for one day. After a medium change to 0.1% BSA basal medium, the cells are incubated with FGF-2 or polypeptides of the invention with or without IL-1(for 24 hours. The supernatants are collected and assayed for PGE2 by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or with or without polypeptides of the invention IL-1(for 24 hours. The supernatants are collected and assayed for IL-6 by ELISA kit (Endogen, Cambridge, Mass.).

Human lung fibroblasts are cultured with FGF-2 or polypeptides of the invention for 3 days in basal medium before the addition of Alamar Blue to assess effects on growth of the fibroblasts. FGF-2 should show a stimulation at 10-2500 ng/ml which can be used to compare stimulation with polypeptides of the invention.

### Parkinson Models

The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP+) and released. Subsequently, MPP+ is actively accumulated in dopaminergic neurons by the high-affinity reuptake transporter for dopamine. MPP+ is then concentrated in mitochondria by the electrochemical gradient and selectively inhibits nicotidamide adenine disphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, polypeptides of the invention can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of a polypeptide of the invention is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm2 on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopaminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if a polypeptide of the invention acts to prolong the survival of dopaminergic neurons, it would suggest that the polypeptide may be involved in Parkinson's Disease.

One skilled in the art could easily modify the exemplified studies to test the activity of polynucleotides of the invention (e.g., gene therapy), agonists, and/or antagonists of polynucleotides or polypeptides of the invention.

The contents of all patents, patent applications, published PCT applications and articles, books, references, reference manuals, abstracts and internet websites cited herein, including the Sequence Listing, are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. An isolated nucleic acid molecule comprising a polynucleotide having a nucleotide sequence selected from the group consisting of:
(a) a polynucleotide fragment of SEQ ID NO:1 or a polynucleotide fragment of the cDNA sequence included in ATCC Deposit No: PTA-3949, which is hybridizable to SEQ ID NO:1;
(b) a polynucleotide encoding a polypeptide fragment of SEQ ID NO:2 or a polypeptide fragment encoded by the cDNA sequence included in ATCC Deposit No: PTA-3949, which is hybridizable to SEQ ID NO:1;
(c) a polynucleotide encoding a polypeptide domain of SEQ ID NO:2 or a polypeptide domain encoded by the cDNA sequence included in ATCC Deposit No: PTA-3949, which is hybridizable to SEQ ID NO:1;
(d) a polynucleotide encoding a polypeptide epitope of SEQ ID NO:2 or a polypeptide epitope encoded by the cDNA sequence included in ATCC Deposit No: PTA-3949, which is hybridizable to SEQ ID NO:1;
(e) a polynucleotide encoding a polypeptide of SEQ ID NO:2 or the cDNA sequence included in ATCC Deposit No: PTA-3949, which is hybridizable to SEQ ID NO: 1, having GPCR activity;
(f) an isolated polynucleotide comprising nucleotides 93 to 1010 of SEQ ID NO: 1, wherein said nucleotides encode a polypeptide corresponding to amino acids 2 to 307 of SEQ ID NO:2 minus the start codon;
(g) an isolated polynucleotide comprising nucleotides 90 to 1010 of SEQ ID NO: 1, wherein said nucleotides encode a polypeptide corresponding to amino acids 1 to 307 of SEQ ID NO:2 including the start codon;
(h) a polynucleotide which represents the complimentary sequence (antisense) of SEQ ID NO:1;
(i) a polynucleotide encoding a polypeptide of SEQ ID NO:4, which is hybridizable to SEQ ID NO:3, having GPCR activity;
(j) an isolated polynucleotide comprising nucleotides 4 to 966 of SEQ ID NO:3, wherein said nucleotides encode a polypeptide corresponding to amino acids 2 to 321 of SEQ ID NO:4 minus the start codon;
(k) an isolated polynucleotide comprising nucleotides 1 to 966 of SEQ ID NO:3, wherein said nucleotides encode a polypeptide corresponding to amino acids 1 to 321 of SEQ ID NO:4 including the start codon;
(l) a polynucleotide which represents the complimentary sequence (antisense) of SEQ ID NO:3; and
(m) a polynucleotide capable of hybridizing under stringent conditions to any one of the polynucleotides specified in (a)-(l), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment consists of a nucleotide sequence encoding a human G-protein coupled receptor.

3. A recombinant vector comprising the isolated nucleic acid molecule of claim 1.

4. A recombinant host cell comprising the vector sequences of claim 3.

5. An isolated polypeptide comprising an amino acid sequence selected from the group consisting of:
(a) a polypeptide fragment of SEQ ID NO:2 or the encoded sequence included in ATCC Deposit No: PTA-3949;
(b) a polypeptide fragment of SEQ ID NO:2 or the encoded sequence included in ATCC Deposit No: PTA-3949, having GPCR activity;
(c) a polypeptide domain of SEQ ID NO:2 or the encoded sequence included in ATCC Deposit No: PTA-3949;
(d) a polypeptide epitope of SEQ ID NO:2 or the encoded sequence included in ATCC Deposit No: PTA-3949;
(e) a full length protein of SEQ ID NO:2 or the encoded sequence included in ATCC Deposit No: PTA-3949;
(f) a polypeptide comprising amino acids 2 to 307 of SEQ ID NO:2, wherein said amino acids 2 to 307 comprising a polypeptide of SEQ ID NO:2 minus the start methionine;
(g) a polypeptide comprising amino acids 1 to 307 of SEQ ID NO:2;
(h) a full length protein of SEQ ID NO:4;
(i) a polypeptide comprising amino acids 2 to 321 of SEQ ID NO:4, wherein said amino acids 2 to 321 comprising a polypeptide of SEQ ID NO:4 minus the start methionine; and
(j) a polypeptide comprising amino acids 1 to 321 of SEQ ID NO:4.

6. The isolated polypeptide of claim 5, wherein the full length protein comprises sequential amino acid deletions from either the C-terminus or the N-terminus.

7. An isolated antibody that binds specifically to the isolated polypeptide of claim 5.

8. A recombinant host cell that expresses the isolated polypeptide of claim 5.

9. A method of making an isolated polypeptide comprising:
(a) culturing the recombinant host cell of claim 8 under conditions such that said polypeptide is expressed; and
(b) recovering said polypeptide.

10. The polypeptide produced by claim 9.

11. A method for preventing, treating, or ameliorating a medical condition, comprising the step of administering to a mammalian subject a therapeutically effective amount of the polypeptide of claim 5, or a modulator thereof.

12. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

13. A method of diagnosing a pathological condition or a susceptibility to a pathological condition in a subject comprising:
(a) determining the presence or amount of expression of the polypeptide of claim 5 in a biological sample; and
(b) diagnosing a pathological condition or a susceptibility to a pathological condition based on the presence or amount of expression of the polypeptide.

14. An isolated nucleic acid molecule consisting of a polynucleotide having a nucleotide sequence selected from the group consisting of:
(a) a polynucleotide encoding a polypeptide of SEQ ID NO:2;
(b) an isolated polynucleotide consisting of nucleotides 93 to 1010 of SEQ ID NO:1, wherein said nucleotides encode a polypeptide corresponding to amino acids 2 to 307 of SEQ ID NO:2 minus the start codon;
(c) an isolated polynucleotide consisting of nucleotides 90 to 1010 of SEQ ID NO:1, wherein said nucleotides encode a polypeptide corresponding to amino acids 1 to 307 of SEQ ID NO:2 including the start codon;
(d) a polynucleotide encoding the HGPRBMY39 polypeptide encoded by the cDNA clone contained in ATCC Deposit No. PTA-3949;
(e) a polynucleotide which represents the complimentary sequence (antisense) of SEQ ID NO:1;
(f) a polynucleotide encoding a polypeptide of SEQ ID NO:4;
(g) an isolated polynucleotide consisting of nucleotides 4 to 966 of SEQ ID NO:3, wherein said nucleotides encode a polypeptide corresponding to amino acids 2 to 321 of SEQ ID NO:4 minus the start codon;
(h) an isolated polynucleotide consisting of nucleotides 1 to 966 of SEQ ID NO:3, wherein said nucleotides encode a polypeptide corresponding to amino acids 1 to 321 of SEQ ID NO:4 including the start codon; and
(i) a polynucleotide which represents the complimentary sequence (antisense) of SEQ ID NO:3.

15. The isolated nucleic acid molecule of claim 14, wherein the polynucleotide comprises a nucleotide sequence encoding a human G-protein coupled receptor.

16. A recombinant vector comprising the isolated nucleic acid molecule of claim 15.

17. A recombinant host cell comprising the recombinant vector of claim 16.

18. An isolated polypeptide consisting of an amino acid sequence selected from the group consisting of:
(a) a polypeptide fragment of SEQ ID NO:2 having GPCR activity;
(b) a polypeptide domain of SEQ ID NO:2 having GPCR activity;
(c) a full length protein of SEQ ID NO:2;
(d) a polypeptide corresponding to amino acids 2 to 307 of SEQ ID NO:2, wherein said amino acids 2 to 307 consisting of a polypeptide of SEQ ID NO:2 minus the start methionine;
(e) a polypeptide corresponding to amino acids 1 to 307 of SEQ ID NO:2;
(f) a polypeptide encoded by the cDNA contained in ATCC Deposit No. PTA-3949;
(g) a full length protein of SEQ ID NO:4;
(h) a polypeptide corresponding to amino acids 2 to 321 of SEQ ID NO:4, wherein said amino acids 2 to 321 consisting of a polypeptide of SEQ ID NO:4 minus the start methionine; and
(i) a polypeptide corresponding to amino acids 1 to 321 of SEQ ID NO:4.

19. The method of diagnosing a pathological condition of claim 15 wherein the condition is a member of the group consisting of: reproductive disorder; a male reproductive disorder; a testicular disorder; testicular cancer; a disorder related to aberrant G-protein coupled signaling; a disorder related to aberrant G-protein coupled signaling, particularly pathways that signal through the G alpha i/o family of G-proteins; a disorder related to aberrant G-protein coupled receptor dependent cAMP signaling; a disorder related to aberrant G-protein coupled receptor dependent signaling associated with CRE elements; an immune disorder; hematopoietic disorder; reproductive disorder; a disorder related to aberrant T-cell maturation; leukemia; multiple myeloma; related proliferative condition of the immune system; neural disorder; brain cancer; related proliferative condition of the central nervous system; hypersensitivity disorders; particularly pain disorders; neural disorder related to either a direct or indirect interaction with voltage-gated sodium channels and their beta subunits; disorders related to aberrations or injuries in the cerebellum, including, but not limited to, cerebellar ataxias of known and unknown origin such as Coeliac disease, and other diseases associated with this region of the brain such as, Rett syndrome, Parkinson disease, von Hippel-Lindau syndrome, familial congenital cerebellar hypoplasia, and dysplastic gangliocytoma of cerebellum; renal disorders; bladder disorders; urinary incontinence; and over-active bladder.

20. The method for preventing, treating, or ameliorating a medical condition of claim 11, wherein the medical condition is selected from the group consisting of a reproductive disorder; a male reproductive disorder; a testicular disorder; testicular cancer; a disorder related to aberrant G-protein coupled signaling; a disorder related to aberrant G-protein coupled signaling, particularly pathways that signal through the G alpha i/o family of G-proteins; a disorder related to aberrant G-protein coupled receptor dependent cAMP signaling; a disorder related to aberrant G-protein coupled receptor dependent signaling associated with CRE elements; an immune disorder; hematopoietic disorder; reproductive disorder; a disorder related to aberrant T-cell maturation; leukemia; multiple myeloma; related proliferative condition of the immune system; neural disorder; brain cancer; related proliferative condition of the central nervous system; hypersensitivity disorders; particularly pain disorders; neural disorder related to either a direct or indirect interaction with voltage-gated sodium channels and their beta subunits; disorders related to aberrations or injuries in the cerebellum, including, but not limited to, cerebellar ataxias of known and unknown origin such as Coeliac disease, and other diseases associated with this region of the brain such as, Rett syndrome, Parkinson disease, von Hippel-Lindau syndrome, familial congenital cerebellar hypoplasia, and dysplastic gangliocytoma of cerebellum; renal disorders; bladder disorders; urinary incontinence; and over-active bladder.

21. A method for treating, or ameliorating a medical condition according to claim 19 wherein the modulator is a member of the group consisting of: a small molecule, a peptide, and an antisense molecule.

22. A method for treating, or ameliorating a medical condition according to claim 20 wherein the modulator is an antagonist.

23. A method for treating, or ameliorating a medical condition according to claim 21 wherein the modulator is an agonist.

24. A method of screening for candidate compounds capable of modulating the activity of a G-protein coupled receptor polypeptide, comprising:
(a) contacting a test compound with a cell or tissue expressing the polypeptide comprising an amino acid sequence as set forth in SEQ ID NO:2 or SEQ ID NO:4; and
(b) selecting as candidate modulating compounds those test compounds that modulate activity of the G-protein coupled receptor polypeptide, wherein said candidate modulating compounds are useful for the treatment of a disorder.

25. The method according to claim 23 wherein said cells are selected from the group consisting of: mammalian cells, CHO cells, CHO-K1 cells, HEK cells, and HEK 293 cells.

26. The method according to claim 24 wherein said cells comprise a vector comprising the coding sequence of the luciferase gene under the control of CRE response elements.

27. The method according to claim 24 wherein said cells comprise a vector comprising the coding sequence of the beta lactamase gene under the control of NFAT response elements.

28. The method according to claim 26 wherein said cells further comprise a vector comprising the coding sequence of G alpha 15 under conditions wherein G alpha 15 is expressed.

29. The method according to claim 27 wherein said cells express a member of the group consisting of: the polypeptide of claim 8 at low levels, the polypeptide of claim 8 at moderate levels, the polypeptide of claim 8 at high levels, beta lactamase at low levels, beta lactamase at moderate levels, and beta lactamase at high levels.

30. The method according to claim 25 wherein said cells express a member of the group consisting of: the polypeptide of claim 8 at low levels, the polypeptide of claim 8 at moderate levels, the polypeptide of claim 8 at high levels, luciferase at low levels, luciferase at moderate levels, and luciferase at high levels.


SEQUENCE LISTING (42 sequences)

SEQ ID NO: 1 - DNA, 3791 bp, Homo sapiens
  CDS (90)..(1010)
ctgaagctggaaattcgaggggcacccaggggtcccccgtggtcccagcaacacagaatttcacattggtacatcttcctctcgtagggatgaaccagactttgaatagcagtgggaccgtggagtcagccctaaactattccagagggagcacagtgcacacggcctacctggtgctgagctccctggccatgttcacctgcctgtgcgggatggcaggcaacagcatggtgatctggctgctgggctttcgaatgcacaggaaccccttctgcatctatatcctcaacctggcggcagccgacctcctcttcctcttcagcatggcttccacgctcagcctggaaacccagcccctggtcaataccactgacaaggtccacgagctgatgaagagactgatgtactttgcctacacagtgggcctgagcctgctgacggccatcagcacccagcgctgtctctctgtcctcttccctatctggttcaagtgtcaccggcccaggcacctgtcagcctgggtgtgtggcctgctgtggacgctctgtctcctgatgaacgggttgacctcttccttctgcagcaagttcttgaaattcaatgaagatcggtgcttcagggtggacatggtccaggccgccctcatcatgggggtcttaaccccagtgatgactctgtccagcctgaccctctttgtctgggtgcggaggagctcccagcagtggcggcggcagcccacacggctgttcgtggtggtcctggcctctgtcctggtgttcctcatctgttccctgcctctgagcatctactggtttgtgctctactggttgagcctgccgcccgagatgcaggtcctgtgcttcagcttgtcacgcctctcctcgtccgtaagcagcagcgccaaccccgccaccaggtccctggggactgtgctccaacaggcgcttcgcgaggagcccgagctggaaggtggggagacgcccaccgtgggcaccaatgagatgggggcttgagagccgcccacaggtgcttcccacctgtgcgagcccatgccctggagattcccgagccgtgagctgcctcccacctcgtccctgccaagtgtctggcccgccttcttgggggagccccaaggactttgcagctgcatgtgggggtcacttccctgcatgtcaaaactccccacaacacctgtgtcctggatctcacaatgcaaccccgctggaagatgcaatttatttgtttatagcagattatctggttgggggaaaatattagcttttagccctaccctgctttaagctggttatctatgggtggataacaggaatctcatttgaatatgaaagctttctcccaaccaccttctctgcctaagacggcatctttgtggaggaggcaggtgacctcgggtgatctttggccctatgctggtccttgggtggcatcaactggccaccaaggtggtgcccccgtgcacctttccccaggtgtgcctgaagtctggcaagtaaacggtgtcgccatcgccgacccggatctcgctggctgtgggccatgagactctgccccaccatccctctgacccctggccagcccttgacccctggcatttgctatgaggtcgccttaccctgtcacccctcgcacccagggcaggcctgttcgctttcctccaggccgcactgggaagggaagagcttctgcctggcttcactggcagtcactccagcagatgctgataggacagtgccccaggcagtggagggcagccttccaggcagtgtcccccagagccagagtctcatacctcactgactgtccctcccagggccccctcactttgcccaaaagcccaaagacggaacatctgggacacacatcagctggctccactctcctccctccccctttccttcccacagtcccctgggtccagaacaaggcagatgtttcctgcactttccacaaatcacagccctttcctaccacccagcatccagcctcgaggctcggggcttgcatggagggggtgcaagacaggaggtccctgcagagtcagtcctggacttaaagaggtgagggaggaggaaagacggaagtggaaagatcctgtctccagaccacagcctggcctgcaagcctggcacctggctgcgtcatcctgtttttcacgtcagaaggtgaaaatgatcttgttctttctcttggcatctttctgaagcagttttcagtctcctcccaaaagatggagccttcgagccagccagggcacaggtcagatcctgagacaggagaggacagctcatcagtaagctataaaatacacccctggaatcaactctcacagcctgcggttctgtgccgtggcaggtgatggtgtcctggggactcgaagactgtgccctggacaggactccccttcctgggctccccctgtttgttcattgccctgggctgggggctgtgcagccctcccagaaggagaaattacctctgagtgctttcccacctagggctcaggcccccaggtctgtgtgtcccagagaagcatgcgaggcagagtggtggctctttctcagtcgttccagcatttagcggagcacttggtaccagcactggcctcaaatgccagatgcacttaacaagtgctcatttggccagtaagagtaactgatgcctgtacagtgctttacagcccacgttttgtccaggatccaacccatatcttctgactacaaaccccatgcttcacaacccttctccaacctgggcagccacgaggtggcccagtctaaagccgacggttggtaggaagaccatctcgcttcttagcactcagtggctaccacagggcccctcatttgcaaggggaggagccctagaaattgttcacacaatgatgttatgtttcgataaaacatataaaatgatatatctgttagaccttctactccggtacctccatcacactccactttaggtcaggtggcactggtgtgcccacaggtatttggggggtggggctgagtggaagaggagttagggggtccctgcagtttgggcttagtgaggtgcatttatgtggtttccgatcacttctgtgtgtggttaggcaattgctagcaccccagggtaggaatggctcccagaaacgttgccactgcctgctgtgccagctccccgggcatcgtgcaaagagctgcctgaaaaaatgaacagataaaggaagtatgtgggggagtgggtaacagagaagaaataagatttgaaatgtacagaatcagaagctagcttatagaagaatcctccaatccccagatgtacaactctaaatggggggttctgttctcacgggtgcctgctcagagcaggtctcacctgttagcaagatccttgctaatgcagcatgtgtaatcagaacacataccatacctcttttctcctgggaaatgtgaaagtaccattcatgagacaccctgtttttgtatccaaaatgctggagcaggaatacaaatggcaaaaacgtctctgtgtgaagttgcatgttttatgtatccagctatcactcacttaaaataaatccaatcaatgaggaaaaacacaattatctttctcttttctccatagaaagtgtacaaaattgatctatgaagaggcaagcaacagtatgcagccaaaaatgtaggacaaatattagaaaggtttgttaagcagctaattattaaaaatattatagggcttttaatattttaaaaaaaaaaaaaaaaaaaaaaaaaaaaaaaaaaaaa

SEQ ID NO: 2 - protein, 307 aa, Homo sapiens
MNQTLNSSGTVESALNYSRGSTVHTAYLVLSSLAMFTCLCGMAGNSMVIWLLGFRMHRNPFCIYILNLAAADLLFLFSMASTLSLETQPLVNTTDKVHELMKRLMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPATRSLGTVLQQALREEPELEGGETPTVGTNEMGA

SEQ ID NO: 3 - DNA, 966 bp, Homo sapiens
  CDS (1)..(963)
atgaaccagactttgaatagcagtgggaccgtggagtcagccctaaactattccagagggagcacagtgcacacggcctacctggtgctgagctccctggccatgttcacctgcctgtgcgggatggcaggcaacagcatggtgatctggctgctgggctttcgaatgcacaggaaccccttctgcatctatatcctcaacctggcggcagccgacctcctcttcctcttcagcatggcttccacgctcagcctggaaacccagcccctggtcaataccactgacaaggtccacgagctgatgaagagactgatgtactttgcctacacagtgggcctgagcctgctgacggccatcagcacccagcgctgtctctctgtcctcttccctatctggttcaagtgtcaccggcccaggcacctgtcagcctgggtgtgtggcctgctgtggacactctgtctcctgatgaacgggttgacctcttccttctgcagcaagttcttgaaattcaatgaagatcggtgcttcagggtggacatggtccaggccgccctcatcatgggggtcttaaccccagtgatgactctgtccagcctgaccctctttgtctgggtgcggaggagctcccagcagtggcggcggcagcccacacggctgttcgtggtggtcctggcctctgtcctggtgttcctcatctgttccctgcctctgagcatctactggtttgtgctctactggttgagcctgccgcccgagatgcaggtcctgtgcttcagcttgtcacgcctctcctcgtccgtaagcagcagcgccaaccccgtcatctacttcctggtgggcagccggaggagccacaggctgcccaccaggtccctggggactgtgctccaacaggcgcttcgcgaggagcccgagctggaaggtggggagacgcccaccgtgggcaccaatgagatgggggcttga

SEQ ID NO: 4 - protein, 321 aa, Homo sapiens
MNQTLNSSGTVESALNYSRGSTVHTAYLVLSSLAMFTCLCGMAGNSMVIWLLGFRMHRNPFCIYILNLAAADLLFLFSMASTLSLETQPLVNTTDKVHELMKRLMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPVIYFLVGSRRSHRLPTRSLGTVLQQALREEPELEGGETPTVGTNEMGA

SEQ ID NO: 5 - DNA, 21 bp, Homo sapiens
ccatgcttcacaacccttctc

SEQ ID NO: 6 - DNA, 21 bp, Homo sapiens
accaaccgtcggctttagact

SEQ ID NO: 7 - protein, 345 aa, Cavia porcellus
MMVTVSYDYDYNSTFLPDGFVDNYVERLSFGDLVAVVIMVVVFLVGVPGNALVVWVTACEARRHINAIWFLNLAAADLLSCLALPILLVSTVHLNHWYFGDTACKVLPSLILLNMYTSILLLATISADRLLLVLSPIWCQRFRGGCLAWTACGLAWVLALLLSSPSFLYRRTHNEHFSFKVYCVTDYGRDISKERAVALVRLLVGFIVPLITLTACYTFLLLRTWSRKATRSAKTVKVVVAVVSSFFVFWLPYQVTGILLAWHSPNSATYRNTKALDAVCVAFAYINCCINPIIYVVAGHGFQGRLLKSLPSVLRNVLTEESLDKRHQSFARSTVDTMPQKSESV

SEQ ID NO: 8 - protein, 349 aa, Pongo pygmaeus
METNFSIPLNESEEVLPEPAGHTVLWIFSLLVHGVTFIFGVLGNGLVIWVAGFRMTRTVNTICYLNLALADFSFSAILPFRMVSVAMREKWPFGTFLCKLVHVMIDINLFVSVYLITIIALDRCICVLHPAWAQNHRTMSLAKRVMMGLWILAIVLTLPNFIFWTTISTKNGDTYCIFNFPFWGDTAVERLNAFITMGKVFLILHFIIGFSMPMSIITVCYGIIAAKIHRNHMIKSSSPLRVFAAVVASFFICWFPYELIGILMAVWLKEMLLNGKYKIILVLLNPTSSLAFFNSCLNPILYVFLGSNFQERLIRSLPTSLERALTEVPDSAQTSNTHTNSASPPEETE

SEQ ID NO: 9 - protein, 321 aa, Mus musculus
MEPLAMTLYPLESTQPTRNKTPNETTWSSEHTDDHTYFLVSLVICSLGLAGNGLLIWFLIFCIKRKPFTIYILHLAIADFMVLLCSSIMKLVNTFHIYNMTLESYAILFMIFGYNTGLHLLTAISVERCLSVLYPIWYQCQRPKHQSAVACMLLWALSVLVSGLENFFCILEVKPQFPECRYVYIFSCILTFLVFVPLMIFSNLILFIQVCCNLKPRQPTKLYVIIMTTVILFLVFAMPMKVLLIIGYYSSSLDDSVWDSLPYLNMLSTINCSINPIVYFVVGSLRRKRSRKSLKEALQKVFEEKPVVASRENVTQFSLPS

SEQ ID NO: 10 - protein, 325 aa, Homo sapiens
MDGSNVTSFVVEEPTNISTGRNASVGNAHRQIPIVHWVIMSISPVGFVENGILLWFLCFRMRRNPFTVYITHLSIADISLLFCIFILSIDYALDYELSSGHYYTIVTLSVTFLFGYNTGLYLLTAISVERCLSVLYPIWYRCHRPKYQSALVCALLWALSCLVTTMEYVMCIDREEESHSRNDCRAVIIFIAILSFLVFTPLMLVSSTILVVKIRKNTWASHSSKLYIVIMVTIIIFLIFAMPMRLLYLLYYEYWSTFGNLHHISLLFSTINSSANPFIYFFVGSSKKKRFKESLKVVLTRAFKDEMQPRRQKDNCNTVTVETVV

SEQ ID NO: 11 - protein, 324 aa, Mus musculus
MDQSNMTSLAEEKAMNTSSRNASLGSSHPPIPIVHWVIMSISPLGFVENGILLWFLCFRMRRNPFTVYITHLSMADISLLFCIFILSIDYALDYELSSGHHYTIVTLSVTFLFGYNTGLYLLTAISVERCLSVLYPIWYTSHRPKHQSAFVCALLCALSCLVTTMEYVMCIDSGEESHSRSDCRAVIIFIAILSFLVFTPLMLVSSSILVVKIRKNTWASHSSKLYIVIMVTIIIFLIFAMPMRVLYLLYYEYWSAFGNLHNISLLFSTINSSANPFIYFFVGSSKKKRFRESLKVVLTRAFKDEMQPRRQEGNGNTVSIETVV

SEQ ID NO: 12 - protein, 324 aa, Rattus norvegicus
MDQSNMTSFAEEKAMNTSSRNASLGTSHPPIPIVHWVIMSISPLGFVENGILLWFLCFRMRRNPFTVYITHLSIADISLLFCIFILSIDYALDYELSSGHYYTIVTLSVTFLFGYNTGLYLLTAISVERCLSVLYPIWYRCHRPKHQSAFVCALLWALSCLVTTMEYVMCIDSGEESHSQSDCRAVIIFIAILSFLVFTPLMLVSSTILVVKIRKNTWASHSSKLYIVIMVTIIIFLIFAMPMRVLYLLYYEYWSTFGNLHNISLLFSTINSSANPFIYFFVGSSKKKRFRESLKVVLTRAFKDEMQPRRQEGNGNTVSIETVV

SEQ ID NO: 13 - protein, 378 aa, Homo sapiens
MVWGKICWFSQRAGWTVFAESQISLSCSLCLHSGDQEAQNPNLVSQLCGVFLQNETNETIHMQMSMAVGQQALPLNIIAPKAVLVSLCGVLLNGTVFWLLCCGATNPYMVYILHLVAADVIYLCCSAVGFLQVTLLTYHGVVFFIPDFLAILSPFSFEVCLCLLVAISTERCVCVLFPIWYRCHRPKYTSNVVCTLIWGLPFCINIVKSLFLTYWKHVKACVIFLKLSGLFHAILSLVMCVSSLTLLIRFLCCSQQQKATRVYAVVQISAPMFLLWALPLSVAPLITDFKMFVTTSYLISLFLIINSSANPIIYFFVGSLRKKRLKESLRVILQRALADKPEVGRNKKAAGIDPMEQPHSTQHVENLLPREHRVDVET

SEQ ID NO: 14 - protein, 343 aa, Rattus norvegicus
MAGNCSWEAHSTNQNKMCPGMSEALELYSRGFLTIEQIATLPPPAVTNYIFLLLCLCGLVGNGLVLWFFGFSIKRTPFSIYFLHLASADGIYLFSKAVIALLNMGTFLGSFPDYVRRVSRIVGLCTFFAGVSLLPAISIERCVSVIFPMWYWRRRPKRLSAGVCALLWLLSFLVTSIHNYFCMFLGHEASGTACLNMDISLGILLFFLFCPLMVLPCLALILHVECRARRRQRSAKLNHVVLAIVSVFLVSSIYLGIDWFLFWVFQIPAPFPEYVTDLCICINSSAKPIVYFLAGRDKSQRLWEPLRVVFQRALRDGAEPGDAASSTPNTVTMEMQCPSGNAS

SEQ ID NO: 15 - protein, 324 aa, Mus musculus
MDQSNMTSLAEEKAMNTSSRNASLGSSHPPIPIVHWVIMSISPLGFVENGILLWFLCFRMRRNPFTVYITHLSIADIYLLFCIFILSIDYALDYELSSGHHYTIVTLSVTFLFGYNTGLYLLTAISVERCLSVLYPIWYRCHRPKHQSAFVCALLWALSCLVTTMEYVMCIDSGEESHSRSDCRAVIIFIAILSFLVFTPLMLVSSTILVVKIRKNTWASHSSKLYIVIMVTIIIFLIFAMPMRVLYLLYYEYWSAFGNLHNISLLFSTINSSANPFIYFFVGSSKKKRFRESLKDVLTRAFKDEMQPRRQEGNGNTVSIETVV

SEQ ID NO: 16 - DNA, 79 bp, Homo sapiens
atcttcctctcgtagggatgaaccagactttgaatagcagtgggaccgtggagtcagccctaaactattccagagggag

SEQ ID NO: 17 - DNA, 21 bp, Homo sapiens
tctcgtagggatgaaccagac

SEQ ID NO: 18 - DNA, 19 bp, Homo sapiens
cacggtcccactgctattc

SEQ ID NO: 19 - DNA, 38 bp, Homo sapiens
gtccccaagcttgcacctctcgtagggatgaaccagac

SEQ ID NO: 20 - DNA, 53 bp, Homo sapiens
cgggatcctacttgtcgtcgtcgtccttgtagtccacggtcccactgctattc

SEQ ID NO: 21 - protein, 14 aa, Homo sapiens
LFVWVRRSSQQWRR

SEQ ID NO: 22 - protein, 13 aa, Homo sapiens
PLVNTTDKVHELM

SEQ ID NO: 23 - protein, 13 aa, Homo sapiens
LTAISTQRCLSVL

SEQ ID NO: 24 - protein, 35 aa, Homo sapiens
GNSMVIWLLGFRMHRNPFCIYILNLAAADLLFLFS

SEQ ID NO: 25 - protein, 36 aa, Artificial Sequence
  PFAM Consensus Sequence.
GNILVIWVICRHKRMRTPTNYFICNLAVADLLFCLT

SEQ ID NO: 26 - protein, 170 aa, Homo sapiens
LMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANP

SEQ ID NO: 27 - protein, 192 aa, Artificial Sequence
  PFAM Consensus Sequence.
FFYMCCYASIFFLCCISIDRYWAICHPMRYRRRMTRPRHAWVMCLVIWVLAFLWSLPPLMFWWCYTHECPNHWNNCNHTWCFIDWPHESWHHWWTWWRYYYICSCIVGFYIPLLVMCFCYCRIYRTLWKAAKMLCVVVVVFFVCWLPYHIFMFMDTFCMHWWMIWTCELECVIPWAYQICVWLAYVNCCLNP

SEQ ID NO: 28 - protein, 35 aa, Homo sapiens; identical to SEQ ID NO: 24

SEQ ID NO: 29 - protein, 36 aa, Artificial Sequence; identical to SEQ ID NO: 25
  PFAM Consensus Sequence.

SEQ ID NO: 30 - protein, 173 aa, Homo sapiens
LMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPVIY

SEQ ID NO: 31 - protein, 195 aa, Artificial Sequence
  PFAM Consensus Sequence.
FFYMCCYASIFFLCCISIDRYWAICHPMRYRRRMTRPRHAWVMCLVIWVLAFLWSLPPLMFWWCYTHECPNHWNNCNHTWCFIDWPHESWHHWWTWWRYYYICSCIVGFYIPLLVMCFCYCRIYRTLWKAAKMLCVVVVVFFVCWLPYHIFMFMDTFCMHWWMIWTCELECVIPWAYQICVWLAYVNCCLNPIIY

SEQ ID NO: 32 - protein, 10 aa, Homo sapiens
MNQTLNSSGT

SEQ ID NO: 33 - protein, 14 aa, Homo sapiens
MNQTLNSSGTVESA

SEQ ID NO: 34 - protein, 14 aa, Homo sapiens
VESALNYSRGSTVH

SEQ ID NO: 35 - protein, 14 aa, Homo sapiens
TQPLVNTTDKVHEL

SEQ ID NO: 36 - protein, 16 aa, Homo sapiens
TLNSSGTVESALNYSR

SEQ ID NO: 37 - protein, 16 aa, Homo sapiens
FTCLCGMAGNSMVIWL

SEQ ID NO: 38 - protein, 16 aa, Homo sapiens
SAWVCGLLWTLCLLMN

SEQ ID NO: 39 - protein, 16 aa, Homo sapiens
CLLMNGLTSSFCSKFL

SEQ ID NO: 40 - protein, 8 aa, Bacteriophage T7
DYKDDDDK

SEQ ID NO: 41 - DNA, 59 bp, Homo sapiens
ggggacaagtttgtacaaaaaagcaggcttcaccatgaaccagactttgaatagcagtg

SEQ ID NO: 42 - DNA, 49 bp, Homo sapiens
ggggaccactttgtacaagaaagctgggtctcaagcccccatctcattg